(19) United States
(12) Patent Application Publication
(10) Pub. No.: US 2020/0323962 A1
(43) Pub. Date: Oct. 15, 2020

(54) LIPOPHILIC PEPTIDE PRODRUGS

(71) Applicant: YISSUM RESEARCH DEVELOPMENT COMPANY OF THE HEBREW UNIVERSITY OF JERUSALEM LTD., Jerusalem (IL)

(72) Inventors: Amnon HOFFMAN, Jerusalem (IL); Chaim GILON, Jerusalem (IL); Joseph FANOUS, Bat Yam (IL); Adi KLINGER, Rishon Lezion (IL)

(21) Appl. No.: 16/646,644

(22) PCT Filed: Sep. 17, 2018
(86) PCT No.: PCT/IL2018/051042

§ 371 Date: Mar. 12, 2020

Related U.S. Application Data
(60) Provisional application No. 62/560,214, filed on Sep. 19, 2017.

(51) Int. Cl. A61K 38/31; A61K 47/18; A61K 47/22

(52) U.S. Cl. CPC A61K 38/31; A61K 47/183; A61K 47/22; A61K 47/18

(57) ABSTRACT

The present invention relates to methods of preparing peptide-based prodrugs having enhanced oral bioavailability and intestinal penetration. Said prodrugs are characterized in improved lipophilicity, reduced electric charge and tendency to undergo biotransformation through enzymatic reaction (e.g. in the blood stream) to form biologically active peptides.

### FIELD OF THE INVENTION

The present invention relates to peptide-based prodrugs having enhanced oral bioavailability and intestinal permeability and to method of their preparation. The prodrugs of the present invention have improved lipophilicity, reduced electric charge and ability to undergo biotransformation through enzymatic reactions to form biologically active peptides at the desired therapeutic location.

### BACKGROUND OF THE INVENTION

Peptides are key players in a variety of physiological and pathological processes and play important roles in modulating various cell functions. However, peptides have unfavorable pharmacokinetic and pharmacodynamic properties, such as rapid metabolism, poor bioavailability and nonselective receptor activation that limit their development into drugs. Consequently, 90% of the medically approved peptide-based drugs are administered through parenteral routes. Hence, one of the most important challenges in developing peptide drugs is the lack of appropriate physicochemical properties that enables the absorption through biological membranes. After oral intake, a peptide-drug encounters multitude digestive enzymes that degrade them into absorbable entities, such as, amino acid, di-peptides and tri-peptide.

Further major physical barriers in oral uptake are the intestinal epithelial cells, which constitute about 80-90% of the cells in the absorptive surface of the intestinal track. Most peptides are too large and polar to pass this barrier and penetrate the intestine.

Several methods were suggested to improve the Drug-Like Properties (DLPs) of peptides. For example, the cycloscan method (Zimmer et. al., Liebigs Ann. der Chemie, vol. 1993, no. 5, pp. 497-501, May 1993) is based on the selection of backbone cyclic peptide(s) from rationally designed combinatorial library with conformational diversity. Another suggested solution includes “spatial screening” end-to-end N-methylated cyclic penta- and hexa-peptides from focused combinatorial libraries with conformational diversity (Chatterjee et. al. Acc. Chem. Res., vol. 41, no. 10, pp. 1331-1342, October 2008).

WO 2014/130949 discloses cyclic DKCLA (Asp-Lys-Cys-Leu-Ala) peptides, derivatives, mimetics, conjugates or antagonists thereof for use in treating or preventing disorders of bone remodeling such as autoimmune diseases. The compounds disclosed, especially the hydrophilic charged peptides, do not possess improved intestinal or cellular permeability.

Another popular method that intends to improve the DLPs of peptides is the prodrug approach. In this approach, the prodrug is a poorly active or inactive compound containing the parental drug that undergoes some in vivo biotransformation through chemical or enzymatic cleavage. The method attempts to deliver of the active compound to its target overcoming pharmacokinetic, pharmacodynamic and toxicology challenges without permanently altering the pharmacological properties of the parental drug.

Simplicio et al. (Molecules, vol. 13, no. 3, pp. 519-547, March 2008) review the published strategies for the production of prodrugs of amines. The review is divided in two main groups of approaches: those that rely on enzymatic activation and those that take advantage of physiological chemical conditions for release of the drugs.

The active drug dabigatran is a very polar, positively charged non-peptide molecule and therefore it has zero bioavailability after oral administration. In the more lipophilic bifunctional prodrug dabigatran etexilate, the two polar groups, the amidinium and the carboxylate moiety, are masked by carbamic acid ester and carboxylic acid ester groups, respectively, which results in better absorption with bioavailability of 7% after oral administration (G. Eisert, et. al. Arterioscler. Thromb. Vasc. Biol., vol. 30, no. 10, pp. 1885-9, October 2010)

There remains an unmet need for, and it would be advantageous to prepare peptide-based drugs, which show enhance bioavailability and intestinal penetration.

### SUMMARY OF THE INVENTION

The present invention provides processes for the preparation of peptide-based prodrugs, and to peptide-based prodrugs, which are formed by these processes. The peptide-based prodrugs reduce the net charge of the parent peptide, preferably to the extent that it is not charged. As a result, in, the resulting prodrugs are more lipophilic, which may lead to their enhanced bioavailability. The charge reduction is generally achieved through modification of some of the charged amino-acid side chains of the parent peptides and/or the charges termini, to chemically neutral moieties. A specific modification introduces the neutral carbamate moiety (—NCO₂R) to the resulting prodrug, masking a positively charged amino group present in the parent peptide. Another modification introduces the neutral ester moiety (—CO₂R) to the resulting prodrug, masking a negatively charged carboxylate in the parent peptide. In some embodiments, the carbamates and/or the esters are derived from primary alcohols (i.e. R is primary), such that the transformation of the prodrug into the active peptide drug is suspended until the molecule crosses the intestinal wall or reaches the target therapeutic location.

The present invention provides, according to one aspect, a process for preparing a peptide-based prodrug, the process comprising:
(a) providing a peptide; and
(b) reacting said peptide with an alkyl chloroformate having the formula ClCO₂R¹, wherein R¹ is a primary alkyl, thereby forming the peptide-based prodrug.

In some embodiments R¹ is n-C₆H₁₃.

In some embodiments the peptide of step (a) comprises at least one nucleophilic nitrogen atom.

In some embodiments the peptide of step (a) comprises at least one —NHR² moiety, wherein said peptide-based prodrug comprises at least one carbamate moiety having the formula —NR²CO₂R¹, wherein R² is selected from hydrogen and a carbon atom of the peptide of step (a).

In some embodiments the peptide of step (a) is a cyclic peptide.

In some embodiments, the cyclic peptide is a backbone-cyclic peptide.

In some embodiments the peptide of step (a) comprises at least one primary amine, wherein said peptide-based prodrug comprises at least one carbamate moiety having the formula —NHCO₂R¹. In some embodiments the at least one primary amine moiety comprises the N-terminal end of the peptide of step (a).

In some embodiments the peptide-based prodrug comprises at least one carbamate moiety having a formula selected from the group consisting of:

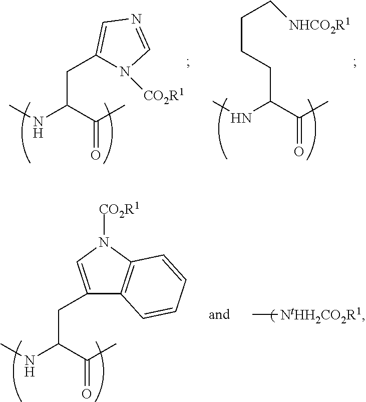

wherein N^{T} is the N-terminal nitrogen atom of the peptide of step (a).

In some embodiments the peptide of step (a) comprises at least one amino acid residue selected from the group consisting of histidine, lysine, tryptophan and combinations thereof.

In some embodiments the peptide-based prodrug comprises at least one carbamate moiety having a formula selected from the group consisting of:

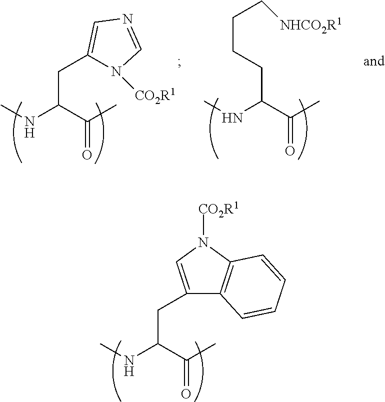

In some embodiments the peptide-based prodrug is having a net neutral charge.

In some embodiments the peptide-based prodrug is devoid of positively charged atoms.

In some embodiments the peptide-based prodrug is devoid of charged atoms.

In some embodiments step (b) is preformed in the presence of a base.

In some embodiments the base is triethylamine.

In some embodiments step (b) is preformed in acetonitrile solvent.

In some embodiments the process further comprises a step of reacting the peptide of step (a) or the peptide-based prodrug of step (b) with an alcohol in the presence of an esterification reagent. In some embodiments the process further comprises step (c) of reacting the peptide-based prodrug with an alcohol in the presence of thionyl chloride.

In some embodiments there is provided a process for preparing a peptide-based prodrug, the process comprising
(a) providing a peptide precursor;
(b) coupling said peptide precursor with a modified amino acid having a formula selected from the group consisting of:

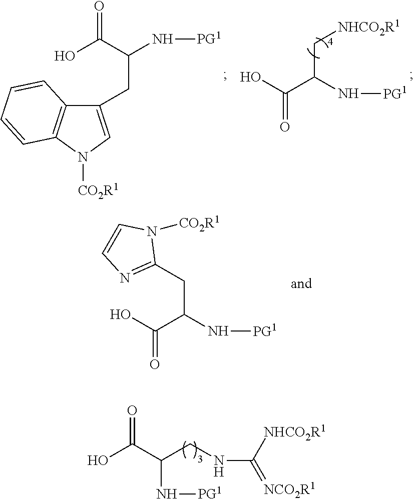

wherein
R¹ is a primary alkyl,
PG¹ is a base-labile protecting group;
wherein the peptide precursor is selected from the group consisting of: an amino acid, a peptide and a solid phase resin.
(c) removing said base-labile protecting group PG¹ from the product of step (b) under basic conditions; and
(d) optionally coupling at least one additional amino acid;
   thereby forming the peptide-based prodrug.

In some embodiments the modified amino acid is having a formula selected from the group consisting of:

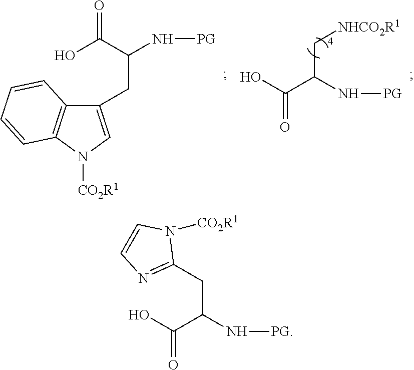

In some embodiments the modified amino acid is having the formula:

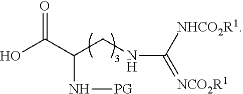

In some embodiments the process further comprises a step of reacting the product of step (c) or (d) with an alkyl chloroformate having the formula ClCO₂R¹. In some embodiments the peptide precursor comprises a terminal primary amino group. In some embodiments the peptide-based prodrug comprises a terminal carbamate moiety having the formula —NHCO₂R¹.

In some embodiments the peptide-based prodrug is a cyclic peptide-based prodrug.

In some embodiments said peptide precursor is a solid phase resin.

In some embodiments said peptide precursor is a solid phase resin having at least one amino acid residue.

In some embodiments the process further comprises a step of removing the peptide-based prodrug from the solid phase resin.

In some embodiments PG¹ is fluorenylmethyloxycarbonyl (Fmoc)

In some embodiments R¹ is n-C₆H₁₃.

In some embodiments the coupling of step (b) comprises contacting said peptide precursor and said modified amino acid in the presence of a coupling agent selected from a carbodiimide, 1-[Bis(dimethylamino)methylene]-1H-1,2,3-triazolo[4,5-b]pyridinium 3-oxid hexafluorophosphate), 1-Hydroxy-7-azabenzotriazole and combinations thereof.

In some embodiments the peptide-based prodrug is having a net neutral charge.

In some embodiments the peptide-based prodrug is devoid of charged atoms.

In some embodiments the peptide-based prodrug is devoid of positively charged atoms.

In some embodiments the process further comprises a step of reacting the peptide of step (a) or peptide-based prodrug of step (b) with an alcohol in the presence of an esterification reagent. In some embodiments the process further comprises the step of reacting the peptide-based prodrug with an alcohol in the presence of thionyl chloride.

In some embodiments there is provided a process for preparing a peptide-based prodrug, the process comprising
(a) providing a peptide precursor;
(b) coupling said peptide precursor with a protected amino acid having a formula selected from the group consisting of:

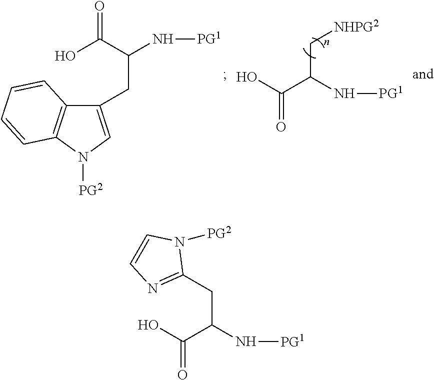

wherein
PG¹ is a base-labile protecting group;
PG² is an acid-labile protecting group;
n is 3 or 4;
wherein the peptide precursor is selected from the group consisting of: an amino acid, a peptide and a solid phase resin;
(c) removing said acid-labile protecting group PG² from the product of step (b) under acidic conditions;
(d) reacting the product of step (c) with a compound having a formula selected from

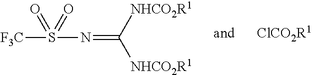

wherein R¹ is a primary alkyl;
(e) removing said base-labile protecting group PG¹ under basic conditions; and
(f) optionally coupling at least one additional amino acid;
   thereby forming the peptide-based prodrug.

In some embodiments the protected amino acid is having the formula

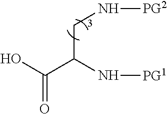

and wherein the reaction of step (d) is with a compound having the formula

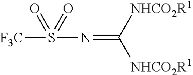

In some embodiments the peptide-based prodrug comprises at least one carbamate moiety having the formula:

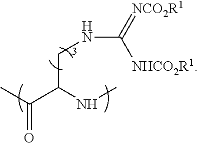

In some embodiments the protected amino acid is having the formula selected from the group consisting of:

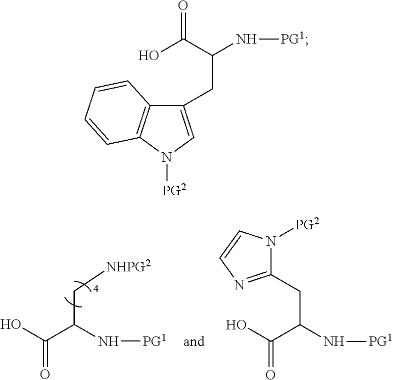

and wherein the reaction of step (d) is with a compound having the formula ClCO₂R¹.

In some embodiments the peptide-based prodrug comprises at least one carbamate moiety having a formula selected from the group consisting of:

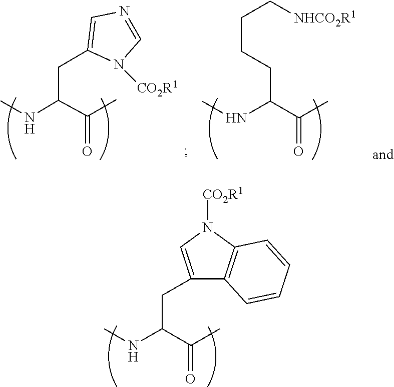

In some embodiments step (b) further comprises removing said base-labile protecting group under basic conditions; and coupling at least one additional amino acid having a second base labile protecting group, wherein step (e) comprises removing said second base-labile protecting group under basic conditions. In some embodiments step (b) further comprises removing said base-labile protecting group under basic conditions; and coupling a plurality of additional amino acids, each having a second base labile protecting group, wherein step (e) comprises removing each of said second base-labile protecting groups under basic conditions.

In some embodiments the acid labile protecting group is 4-methyltrityl (Mtt)

In some embodiments R¹ is n-C₆H₁₃.

In some embodiments the peptide-based prodrug is devoid of charged atoms.

In some embodiments step (d) is preformed in the presence of a base selected from trimethylamine and N,N-Diisopropylethylamine.

In some embodiments the process further comprises a step of reacting the peptide of step (a) or peptide-based prodrug of step (e) or step (f) with an alcohol in the presence of an esterification reagent. In some embodiments the process further comprises step (g) of reacting the peptide-based prodrug with an alcohol in the presence of thionyl chloride.

In some embodiments the process further comprises a step of reacting the product of step (e) or (f) with an alkyl chloroformate having the formula ClCO₂R¹. In some embodiments said peptide precursor comprises a terminal primary amino group. In some embodiments the peptide-based prodrug comprises a terminal carbamate moiety having the formula —NHCO₂R¹.

In some embodiments the peptide-based prodrug is a cyclic peptide-based prodrug.

In some embodiments said peptide precursor is a solid phase resin.

In some embodiments said peptide precursor is a solid phase resin having at least one amino acid residue.

In some embodiments the process further comprises a step of removing the peptide-based prodrug from the solid phase resin.

In some embodiments PG¹ is fluorenylmethyloxycarbonyl (Fmoc).

In some embodiments the coupling of step (b) comprises contacting said peptide precursor and said protected amino acid in the presence of a coupling agent selected from a carbodiimide, 1-[Bis(dimethylamino)methylene]-1H-1,2,3-triazolo[4,5-b]pyridinium 3-oxid hexafluorophosphate), 1-Hydroxy-7-azabenzotriazole and combinations thereof.

The present invention also provides a peptide-based prodrug comprising at least one -carbamate moiety, wherein said at least one carbamate moiety is selected from the group consisting of:

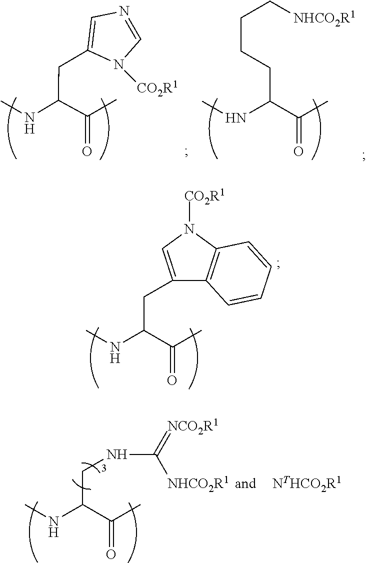

wherein
R¹ is a primary alkyl; and
N^{T} is the peptide's terminal nitrogen atom.

In some embodiments the peptide-based prodrug is a cyclic peptide-based prodrug. In some embodiments the peptide-based prodrug is a cyclic peptide-based prodrug having at least one internal disulfide bond. In some embodiments, the cyclic peptide-based prodrug comprises a backbone cyclization. In some embodiments the peptide-based prodrug is somatostatin or a somatostatin analog.

In some embodiments there is provided a cyclic peptide-based prodrug comprising at least one carbamate moiety, wherein said at least one carbamate moiety is selected from the group consisting of:

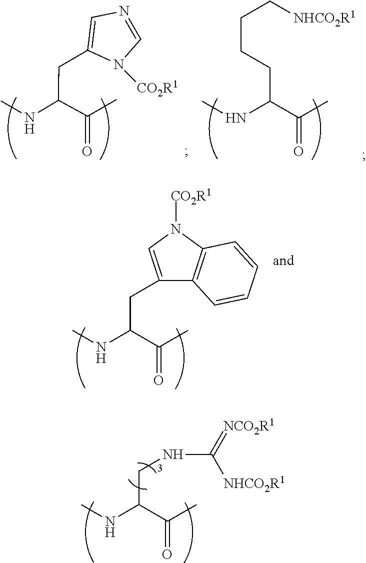

wherein R¹ is a primary alkyl.

Further embodiments, features, advantages and the full scope of applicability of the present invention will become apparent from the detailed description and drawings given hereinafter. However, it should be understood that the detailed description, while indicating preferred embodiments, of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C is a proposed mechanistic flowchart for gastrointestinal pathway for a peptide drug (FIG. 1A); for a BOC charged masked peptide prodrug (FIG. 1B); and for a Hoc-charged masked peptide prodrug (FIG. 1C).

FIG. 2A is a flowchart depicting the development of orally available RGD containing N-methylated (NMe) cylohexapeptides. Abbreviations of amino acids are according to [9]. D-amino acids are represented as the one letter abbreviation but in small letter format. “a” is D-Ala; “r” is D-Arg; “d” is D-Asp. The D amino acid always acquires position 1 and is written on the left. N-methylated amino acids are represented by a superscripted star on the left side of the one letter abbreviation. Thus, NMe Ala is *A, NMe D-Ala is *a, NMe Arg is *R, NMe D-Arg is *r, NMe Asp is *D, NMe D-Asp is *d, NMe Trp is *W, NMe D-Trp is *w, NMe Phe is *F, NMe D-Phe is *f, NMe Val is *V, and NMe D-Val is *v. Hoc is hexyloxycarbonyl. Thus, Arg, which is substituted by two hexyloxycarbonyl groups is R(Hoc)₂ and N-Me D-Arg, which is substituted by two hexyloxycarbonyl groups is *r(Hoc)₂. Aspartic acid esterified by methyl is D(OMe).

FIG. 2B shows structure-permeability relationship (SPR) of some of the members of the N-methylated cyclic Ala hexapeptides. The structures of the four highly Caco-2 permeable di-N-Methylated cyclic hexa-alanine peptide scaffolds (peptides #1, 2, 3, 4) are shown on the right.

FIG. 3A-3B show the structures of peptide 12 (c(*aRGDA*A) SEQ ID NO: 2) (FIG. 3A) and its prodrug peptide 12P (c(*aR(Hoc)₂GD(OMe)A*A) SEQ ID NO: 10) (FIG. 3B).

FIG. 4 shows the Caco-2 apparent permeability coefficient (Papp) of peptide 12 (SEQ ID NO: 2) and peptide 12P (SEQ ID NO: 10). (average±SEM, n=3). Unpaired t-test, ** p<0.005.

FIG. 5 shows the Caco-2 A-to-B and the B-to-A permeability of peptide 12P (SEQ ID NO: 10) (average±SEM, n=3). Unpaired t-test, *** p<0.0005.

FIG. 6 shows the Caco-2 Papp efflux ratios (Papp BA/Papp AB) of Peptide 12P (SEQ ID NO: 10), cyclosporine A and metoprolol.

FIG. 7 shows the Caco-2 Papp of Peptide 12P (SEQ ID NO: 10) A-to-B in the presence of verapamil (100 μM) (average±SEM, n=3). Unpaired t-test, * p<0.05.

FIG. 8 shows the Caco-2 Papp, A-B and B-A as indicated, of peptide 12P (SEQ ID NO: 10) alone or with PC (n=3 for each group). (*) A significant difference was found between Pₐₚₚ AB and BA of peptide 12P (SEQ ID NO: 10) alone (P<0.05).

FIG. 9A-9B show the metabolic stability of Peptide 12 (SEQ ID NO: 2) (FIG. 9A) and Peptide 12P (SEQ ID NO: 10) (FIG. 9B) in rat plasma (average±SEM).

FIG. 10 shows the metabolic stability of peptide 12 (SEQ ID NO: 2) and peptide 12P (SEQ ID NO: 10) in rat BBMVs (average±SEM).

FIG. 11 shows the metabolic stability of Peptide 12P (SEQ ID NO: 10) in the presence of humane liver microsomes (average±SEM) and with Cyp inhibitor (0.1 μM ketoconazole) and PNL formulation.

FIG. 12 shows plasma concentrations plotted against time scale after 5 mg/kg oral administration of Peptide 12P (SEQ ID NO: 10) (n=3) and Peptide 12 (SEQ ID NO: 2) (n=4).

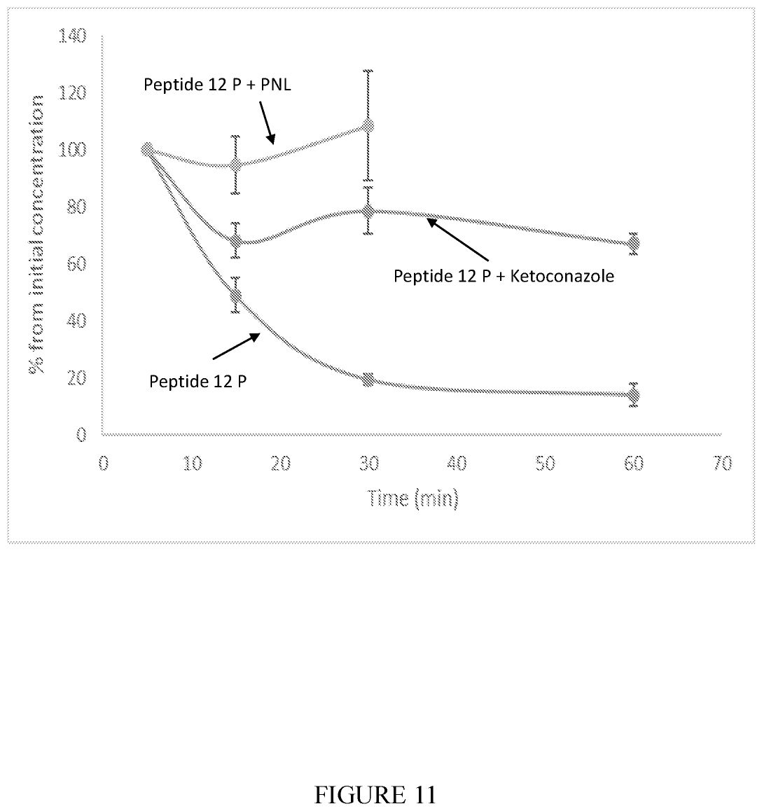

FIG. 13 shows peptide 12P (SEQ ID NO: 10) concentrations following 30 min incubation of dispersed 12P SNEDDS vs. 12P with ketoconazole and 12P alone in isolated rat CYP3A4 microsomes. (n=3 for each group). Significant difference (p<0.01) was found between 12P and dispersed 12P with SNEDDS and between 12P and 12P with ketoconazole (P<0.05).

FIG. 14 shows profiles of plasma concentration of peptide 12 (SEQ ID NO: 2) vs. time in rats after oral administration of 5 mg/kg peptide 12P-SNEDDS and peptide 12. (n=3 for each group).

FIG. 15 shows semi-logarithmic plot of plasma concentration of peptide 12 (SEQ ID NO: 2) vs. time profiles in rats following oral administration of 5 mg/kg of peptides 12P (SEQ ID NO: 10) and 12 and following 0.5 mg/kg bolus administration of peptide 12 (marked as 12 IV), (n=3 for each group).

FIG. 16A-16B show the structures of peptide 29 (c(*vRGDA*A), SEQ ID NO: 5) (FIG. 16A) and peptide 29P (c(*vR(Hoc)₂GD(OMe)A*A), SEQ ID NO: 9) (FIG. 16B).

FIG. 17 shows the Caco-2 A-to-B Papp of Peptide 29P (SEQ ID NO: 9), Peptide 29 (SEQ ID NO: 5) and atenolol. (average±SEM, n=3). Unpaired t-test, ** p<0.005.

FIG. 18 shows the Caco-2 Papp of Peptide 29P (SEQ ID NO: 9): A-to-B vs. B-to-A Papp (average±SEM, n=3). Unpaired t-test, *** p<0.0005.

FIG. 19 shows the Caco-2 Papp of peptide 5 (SEQ ID NO: 1) and peptide 5P (SEQ ID NO: 11) compared to atenolol (average ±SEM, n=3).

FIG. 20 Shows the permeability of peptide 5P (SEQ ID NO: 11) A-to-B vs. B-to-A (average ±SEM, n=3). Unpaired t-test, ** p<0.005.

FIG. 21A-21B Show NMR analysis of peptide 29 (SEQ ID NO: 5) and its prodrug (SEQ ID NO: 11). FIG. 21A is a stereo view of the solution state NMR conformation of 29 superimposed with the conformation of its orally available parent compound. For the sake of clarity, non-polar hydrogens are not shown. FIG. 21B shows binding mode of 29 to the αvβ3 integrin. Receptor amino acid side chains important for the ligand binding are represented as sticks.

FIG. 22 show the structures of peptide 29 (SEQ ID NO: 5), 29P (SEQ ID NO: 9) and their derivatives as well as examined control molecules.

FIG. 23 depicts the synthetic pathway for the preparation of the prodrug hexyloxycarbonyl octreotide (Octreotide-P) from octreotide (SEQ ID NO: 25).

FIG. 24 shows the structures of the peptide analog Somato8 (SEQ ID NO: 26) and its prodrug Somato8-P.

FIG. 25 shows the structures of backbone cyclic somatostatin analogs. A. PTR-3173 (SEQ ID NO: 27), B. PTR-3046 (SEQ ID NO: 28) and C. PTR-3205 (SEQ ID NO: 29).

FIG. 26 shows the structures of the somatostatin analog Somato3M (SEQ ID NO: 30) and its prodrug Somato3M-P.

### DETAILED DESCRIPTION OF THE INVENTION

The present disclosure is directed to various synthetic processes for the preparation of prodrugs of peptides. In some embodiments, said prodrugs are generally characterized by two main chemical features: (a) reduction or omission of electrically charged atoms in the peptide sequence, e.g. through charge masking of charged amino acid residues and terminal amino and carboxylate moieties; and (b) improved lipophilicity provided through introduction of lipophilic groups. A further feature presented by peptide-based prodrugs prepared according to some embodiments of the present processes is their lability in the presence of enzymes in the blood stream or target tissue, which transform the prodrugs into charged biologically active peptide drugs.

A common feature to the processes disclosed herein, according to some embodiments, is the modification of amino acids and/or amino acid residues to their modified counterparts, which include an ester(s) and/or carbamate(s) of primary alcohols. In some embodiments and generally, amino side chains having amine moieties are transformed into carbamates having —NCO₂R fragments; whereas amino side chains having carboxylate moieties are transformed into esters having —CO₂R moieties. In some embodiments, since the esters and amines are of primary alcohols, R is primary, i.e. the first group covalently bonded to the carbonyl's α-sp³ oxygen is a methylene group.

The present invention is based in part on the finding that unlike tertiary carbamates, primary carbamates do not transform into their corresponding amines or ammonium ions until after penetrating through the intestinal wall to the blood stream and/or the lymphatic system. Without wishing to be bound by any theory or mechanism of action, the commonly used tertiary carbamates (e.g. compounds having the tert-butyloxycarbonyl-amino, N—CO₂CMe₃ moiety, N—BOC) undergo O—CMe₃ bond cleavages in gastrointestinal pH. In contrast, primary alkyl carbamates are relatively stable until after penetrating the intestinal wall. Therefore, tertiary carbamates undergo O—CMe₃ bond cleavage before reaching the target therapeutic location (typically in the intestines), to form the corresponding carbamic acids (having —N—CO₂H fragments), which undergo spontaneous decarboxylation to form amines, with
Said amines are then being protonated under physiological or gastrointestinal pH to form charged peptides, which undergo degradation before reaching the target therapeutic location. On the other hand, it was surprisingly found that a similar sequence of reactions, occurs with primary carbamates only after penetrating through the intestines to the blood stream and/or lymphatic system, where the peptide-based drug is most active. It is hypothesized that the difference stems from the high tendency of tertiary carbamates to dissociate under acidic conditions (as the dissociation products include stable tertiary carbocations), while primary carbamates tend to cleave in the presence of esterases, which target and break the O—CH₂ or the carbonyl-OCH₂ bond at the target therapeutic location.

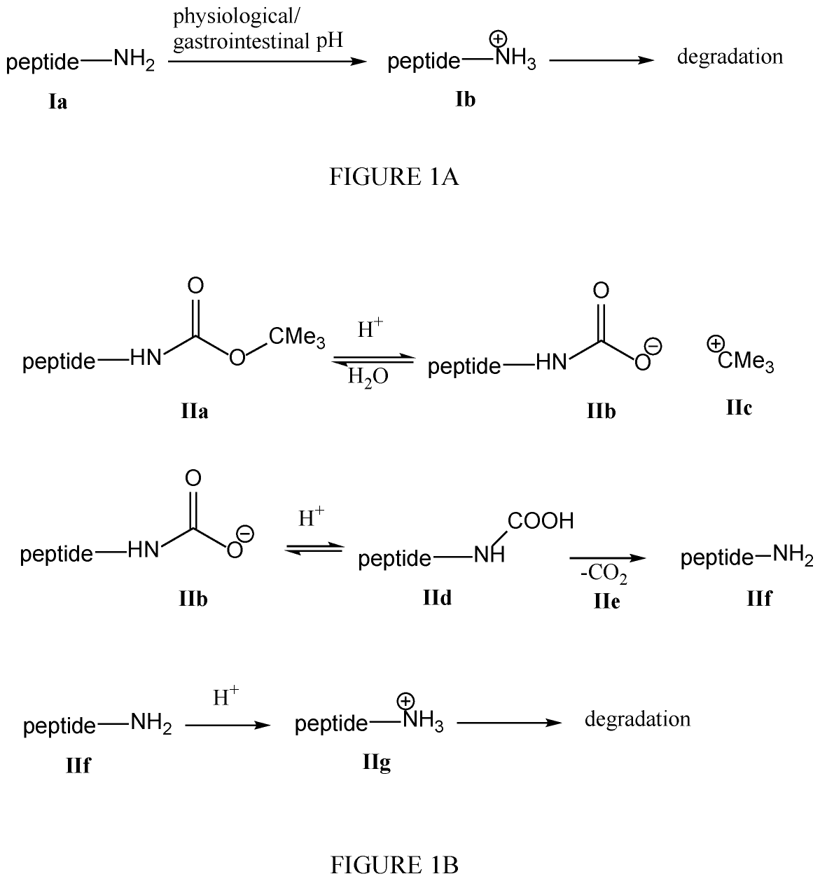

For clarification, reference is made to FIGS. 1A-C which explain the paths of different peptide derivatives, without wishing to be bound by any theory or mechanism of action. FIG. 1A refers to a peptide drug Ia, which penetrates the gastrointestinal tract. Since peptide drug Ia encounters a relatively high concentration of protons, and since it includes basic nitrogen atom(s) (i.e. the terminal NH₂ group, a lysine side chain, and/or a histidine side chain), peptide drug Ia is protonated to become charged peptide drug Ib. Since charged molecules tend to quickly degrade in the GI tract, charged peptide drug Ib undergoes degradation, prior to reaching the intestines. Thus, peptide drug Ia cannot complete its intended biological and/or therapeutic purpose. FIG. 1B refers to a BOC (tert-butyloxycarbonyl) masked peptide prodrug IIa, which penetrates the gastrointestinal tract. Since BOC masked peptide prodrug IIa encounters a relatively high concentration of protons, and since it includes a stable tertiary carbocation fragment, tert-butyl carbocation IIc, it is in equilibrium with its dissociation products—stable tert-butyl carbocation IIc and peptide carbamate anion IIb. In the presence of protons, peptide carbamate anion IIb undergoes protonation to form peptide carbamic acid IId, which, in its turn, undergoes rapid decarboxylation to form carbon dioxide IIe and peptide drug IIf. Thereafter, peptide drug IIf goes in a similar path as peptide drug Ia of FIG. 1A, and degrades through charged peptide drug IIg. Thus, BOC masked peptide prodrug IIa cannot complete its intended biological and/or therapeutic purpose. FIG. 1C refers to a Hoc (Hexyloxycarbonyl) masked peptide prodrug IIIa, which penetrates the gastrointestinal tract. Hoc masked peptide prodrug IIIa again encounters a relatively high concentration of protons. However, it includes a non-stable carbocation primary fragment (n-hexyl primary carbocation). Thus, Hoc masked peptide prodrug IIIa is not in equilibrium with its dissociation products. Rather, Hoc masked peptide prodrug IIIa is stable and may penetrate the intestines through the intestinal lumen. The penetration is further facilitated by the lipophilicity of the hexyl chain of the Hoc masked peptide prodrug IIIa Inside the intestines, Hoc masked peptide prodrug IIIa encounters esterases, which may cut primary esteric bonds. Thus, upon penetration through the intestinal lumen, Hoc masked peptide prodrug IIIa undergoes de-esterification to form peptide carbamic acid IIIb, which, in its turn, undergoes rapid decarboxylation to form carbon dioxide IIIc and peptide drug IIId. Since the active form of Hoc masked peptide prodrug IIIa (i.e. peptide drug IIId) is formed only after penetrating to the blood stream or lymphatic system.

In some embodiments, some the processes disclosed herein are distinctive in the stage in which the modification occurs. Whereas in some of the processes a modification is performed on an amino acid prior to its incorporation to the prodrug in a peptide synthesis; in some processes the modification is performed on an amino acid residue during the peptide synthesis; and in some of the processes the modification is preformed after the completion of the peptide synthesis.

The term “prodrug” refers to a compound which provides an active compound following administration to the individual in which it is used, by a chemical and/or biological process inside the target therapeutic location (e.g., by hydrolysis and/or an enzymatic conversion). The prodrug itself may be active, or it may be relatively inactive, then transformed into a more active compound.

The term “carbamate” as used herein alone or in combination refers to a chemical group or moiety represented by the general structure —N(CO)O—. Carbamate esters may have alkyl or aryl groups substituted on the oxygen.

It is to be understood that when referring to “—NCO₂R” and/or “—NCO₂R fragments” refer to fragments of a molecule. Thus, although neutral nitrogen atoms typically form three bonds, the NCO₂R fragment is portrayed with less bonds, emphasizing the N—C bond between the carbonyl carbon and the nitrogen, which form the carbamate moiety. It is to be understood that the nitrogen is covalently linked to other atoms of the parent peptide, typically carbon and/or hydrogen.

In some embodiments, there is provided a process for preparing a peptide-based prodrug, the process comprising:
(a) providing a peptide; and
(b) reacting said peptide with an alkyl haloformate having the formula XCO₂R¹, wherein R¹ is a primary alkyl and X is a halogen, thereby forming the peptide-based prodrug.

In some embodiments, X is selected from chlorine and bromine. In some embodiments, X is chlorine.

In some embodiments, there is provided a process for preparing a peptide-based prodrug, the process comprising:
(a) providing a peptide; and
(b) reacting said peptide with an alkyl chloroformate having the formula ClCO₂R¹, wherein R¹ is a primary alkyl, thereby forming the peptide-based prodrug.

In some embodiments, there is provided a peptide-based prodrug, prepared by a process comprising:
(a) providing a peptide; and
(b) reacting said peptide with an alkyl chloroformate having the formula ClCO₂R¹, wherein R¹ is a primary alkyl, thereby forming the peptide-based prodrug.

In some embodiments and generally, peptides prepared by the process above are characterized by having a lipophilic CO₂R¹ fragment(s). Specifically, nucleophilic amine moiety or moieties within the skeleton of the starting peptide (i.e. the peptide of step (a)) may be reactive towards chloroformates, forming a lipophilic —NCO₂R¹ fragment(s). In some embodiments the nucleophilic amine moiety or moieties are derived from fragments selected from the group consisting of the amino terminus of the starting peptide, an amino moiety of a histidine side chain, an amino moiety of a tryptophan side chain, an amino moiety of a lysine side chain and combinations thereof.

In some embodiments R¹ is a primary alkyl group.

The term “primary alkyl group” as used herein, refers to an alkyl group, including substituted alkyl groups, unsubstituted alkyl groups, linear alkyl groups, and branched alkyl groups, so long that its first carbon atom is primary. With reference to ClCO₂R¹, NCO₂R¹, NR²CO₂R¹, CO₂R¹ and similar groups, whereupon a primary alkyl is covalently connected to an oxygen atom, “primary alkyl group” comprises a methylene group bonded to the α-sp³ oxygen.

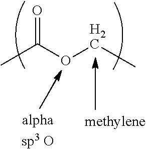

In some embodiments the primary alkyl group, R¹, is selected from substituted primary alkyl, unsubstituted primary alkyl, linear primary alkyl, branched primary alkyl, primary alkylaryl, substituted primary alkylaryl, unsubstituted primary alkylaryl, linear primary alkylaryl, branched primary alkylaryl, primary arylalkyl, substituted primary arylalkyl, unsubstituted primary arylalkyl, linear primary arylalkyl, and branched primary arylalkyl, wherein heteroatoms either may or may not be present in the alkyl group. Each possibility represents a separate embodiment

In some embodiments it is preferable that the primary alkyl group, R¹, does not form a stable carbocation (i.e. [R¹]⁺ is nor stable), as it is hypothesized that increasing the stability of the carbocation may promote the removal of the pro-moiety prior to the prodrug reaching the blood stream. Other than tertiary carbocation, benzyl and allyl carbocations are also considered stable, thus, according to some embodiments it is preferable that the primary alkyl is other than a primary benzyl or allyl.

In some embodiments R¹ is a primary alkyl group, with the proviso that R¹ is not a moiety selected from CH₂—Ar, CH₂-HetAr and CH₂-vinyl. Each possibility represents a separate embodiment. In some embodiments R¹ is a primary alkyl group, with the proviso that R¹ is not a primary benzyl group.

The term “benzyl” as used herein, refers to a —CH₂-aryl group.

The terms “aryl” and “Ar” as used herein, are interchangeable and refer to aromatic groups, such as phenyl, naphthyl and phenanthrenyl, which may optionally contain one or more substituents, such as alkyl, alkoxy, alkylthio, halo, hydroxy, amino and the like.

The terms “heteroaryl” and “HetAr” are interchangeable and refer to unsaturated rings of 5 to 14 atoms containing at least one O, N or S atoms. Heteroaryl may optionally be substituted with at least one substituent, including alkyl, aryl, cycloalkyl, alkoxy, halo amino and the like. Non-limiting examples of heteroaryls include furyl, thienyl, pyrrolyl, indolyl and the like.

The term “vinyl” as used herein, refers to the ethene group —CH═CH₂, which may be substituted or unsubstituted. It may be combined with other groups to provide larger groups such as vinyl ether R—O—CH═CH—, where R is a may include but not limited to alkylene, alkenylene, arylene, and the like; vinyl ketone R(C═O)—CH═CH—, and the like.

Appropriately the alkyl chloroformate may be having an alkyl as described above according to some embodiments. In some embodiments, the peptide-based prodrug comprises said alkyl group. Specifically, in some embodiments, the peptide-based prodrug comprises at least one NR²CO₂R¹ moiety.

In some embodiments the peptide-based prodrug comprises at least one carbamate moiety having a formula selected from the group consisting of:

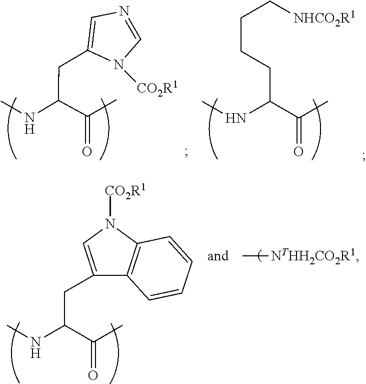

wherein N^{T} is the N-terminal nitrogen atom of the peptide of step (a).

In some embodiments R² is selected from hydrogen and a carbon atom of the peptide of step (a). In some embodiments R² is hydrogen. In some embodiments R² is a carbon atom of the peptide of step (a). For example, in the case that the reactant peptide comprises a lysine residue, a reaction with as described with ClCO₂R¹ may lead to a peptide having a fragment having the formula:

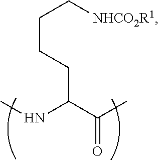

in which R² is H, i.e. the product peptide-based prodrug comprises at least one NHCO₂R¹ moiety. Alternatively, in the case that the reactant peptide comprises a histidine residue, a reaction with as described with ClCO₂R¹ may lead to a peptide having a fragment having the formula:

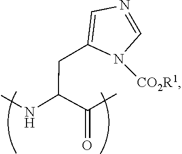

in which R² is a carbon atom of the histidine's side chain, i.e. the product peptide-based prodrug comprises at least one NR²CO₂R¹ moiety, wherein R² is a carbon atom of the peptide of step (a). Similarly, in the case that the reactant peptide comprises a tryptophan residue, a reaction with as described with XCO₂R¹ may lead to a peptide having a fragment having the formula:

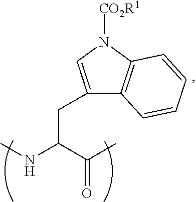

in which R² is a carbon atom of the tryptophan's side chain, i.e. the product peptide-based prodrug comprises at least one NR²CO₂R¹ moiety, wherein R² is a carbon atom of the peptide of step (a).

In some embodiments R¹ is a primary C₃₋₄₀ alkyl. In some embodiments R¹ is a primary C₄₋₃₀ alkyl. In some embodiments R¹ is a primary C₃₋₂₀ alkyl. In some embodiments R¹ is a primary C₃₋₁₂ alkyl. In some embodiments R¹ is a primary C₄₋₂₀ alkyl. In some embodiments R¹ is a primary C₅₋₂₀ alkyl. It is to be understood by a person skilled in the art that “C_{x-y}” alkyl refers to an alkyl group as defined above, which has between x and y carbon atoms. For example C₅₋₂₀ alkyl may include, but not limited to, C₅H₁₁, C₆H₁₃, C₈H₁₇, C₁₀H₂₁, C₁₂H₂₅, C₁₄H₂₉, C₂₀H₄₁ and the like.

In some embodiments R¹ is a straight-chain alkyl. In some embodiments R¹ is an unsubstituted alkyl. In some embodiments R¹ is n-CₙH₂ₙ₊₁, wherein n is in the range of 3 to 15 or 5 to 12. In some embodiments R¹ is n-C₆H₁₃. In some embodiments R¹ is n-C₁₄H₂₉.

In some embodiments the peptide of step (a) is a cyclic peptide. In some embodiments the peptide based prodrug is a cyclic peptide based prodrug. In some embodiments the process further comprises a step of cyclizing the peptide-based prodrug to form a cyclic peptide based prodrug.

As used herein, the term “peptide” is well-known in the art, and is used to refer to a series of linked amino acid molecules. The term is intended to include both short peptide sequences, such as, but not limited to a tripeptide, and longer protein sequences, such as polypeptides and oligopeptides. The term also includes peptide hybrids. The term “hybrid” as used herein refers to amino acid containing oligomers and polymers having at least one other type of monomer. For example, hybrid oligomers may include saccharide(s), nucleoside(s) and/or nucleotide(s), in addition to the amino acid(s) as building block monomers. The terms “peptide-prodrug” and “peptide-base prodrug” are interchangeable and refer to a prodrug variation of a peptide, as termed herein.

The term “cyclic peptide” as used herein refers to a peptide having an intramolecular bond between two non-adjacent amino acids. The cyclization can be effected through a covalent or non-covalent bond, or bridge. Intramolecular bridges include, but are not limited to, backbone to backbone bridge, side-chain to backbone bridge and side-chain to side-chain bridge. The terms “cyclic peptide-prodrug” and “cyclic peptide-base prodrug” are interchangeable and refer to a prodrug variation of a peptide, as termed herein.

In some embodiments the cyclic peptide has a backbone to backbone intramolecular bridge. In some embodiments the cyclic peptide has a head to tail intramolecular bridge. In some embodiments the cyclic peptide has a backbone to backbone head to tail intramolecular bridge. In some embodiments the cyclic peptide has a backbone to backbone intramolecular bridge between the N-terminus and the C-terminus of the peptide. In some embodiments the cyclic peptide-based prodrug has a backbone to backbone intramolecular bridge. In some embodiments the cyclic peptide-based prodrug has a backbone to backbone intramolecular bridge between the N-terminus and the C-terminus of the peptide.

In some embodiments the cyclic peptide has a backbone to side-chain intramolecular bridge. In some embodiments the cyclic peptide-based prodrug has a backbone to side-chain intramolecular bridge.

In some embodiments the cyclic peptide has a side-chain to side-chain intramolecular bridge. In some embodiments the cyclic peptide has a side-chain to side-chain intramolecular disulfide bridge between the cysteine side chain residues. In some embodiments the cyclic peptide-based prodrug has a side-chain to side-chain intramolecular bridge. In some embodiments the cyclic peptide-based prodrug has a side-chain to side-chain intramolecular disulfide bridge between two cysteine side chain residues.

In some embodiments the cyclic peptide is somatostatin or a somatostatin analog.

In some embodiments the cyclic peptide comprises at least one amino acid residues selected from arginine, glycine, aspartic acid and alanine. In some embodiments the cyclic peptide comprises at least two amino acid residues selected from arginine, glycine, aspartic acid and alanine. In some embodiments the cyclic peptide comprises at least three amino acid residues selected from arginine, glycine, aspartic acid and alanine. In some embodiments the cyclic peptide comprises arginine, glycine, aspartic acid and alanine amino acid residues.

In some embodiments the cyclic peptide comprises at least one amino acid residue selected from arginine, glycine and aspartic acid. In some embodiments the cyclic peptide comprises at least two amino acid residues selected from arginine, glycine and aspartic acid. In some embodiments the cyclic peptide comprises arginine, glycine and aspartic acid amino acid residues.

In some embodiments the peptide of step (a) comprises at least one nucleophilic nitrogen atom.

The term “nucleophilic nitrogen atom” refers to a nitrogen atom within an organic compound, which is reactive towards electrophiles under relatively mild conditions. Electrophiles includes, but are not limited to, alkyl haloformates.

In some embodiments the nucleophilic nitrogen atom is reactive towards the alkyl chloroformate in the presence of trimethylamine at 25° C.

In some embodiments the peptide of step (a) comprises at least one —NHR² moiety. In some embodiments the peptide-based prodrug comprises at least one carbamate moiety having the formula —NR²CO₂R¹. In some embodiments the peptide of step (a) comprises at least one —NHR² moiety, wherein said peptide-based prodrug comprises at least one carbamate moiety having the formula —NR²CO₂R¹.

In some embodiments the at least one —NHR² moiety comprises at least one primary amine moiety. In some embodiments the peptide-based prodrug comprises at least one carbamate moiety having the formula —NR²CO₂R¹. In some embodiments the at least one —NHR² moiety is selected from the group consisting of the amino terminus of the peptide of step (a), a histidine side chain, an a tryptophan side chain, a lysine side chain and combinations thereof. In some embodiments the at least one —NHR² moiety is selected from the group consisting of a histidine side chain, a tryptophan side chain, a lysine side chain and combinations thereof. In some embodiments the peptide of step (a) comprises at least one histidine residue. In some embodiments the peptide of step (a) comprises at least one tryptophan residue. In some embodiments the peptide of step (a) comprises at least one lysine residue.

The term “primary amine moiety” refers to the NH₂ group. The term “primary amine” refers to a compound comprising at least one NH₂ group.

In some embodiments the at least one primary amine moiety comprises the N-terminal end of the peptide of step (a).

Specifically, in some embodiments the peptide of step (a) is an unmodified starting peptide. As said starting peptide is unmodified it may include a terminal primary amine moiety, which is being protonated in gastrointestinal/physiological pH. In some embodiments reacting said peptide with an alkyl chloroformate having the formula ClCO₂R¹ results in a formation of an electronically neutral —NR²CO₂R¹ group, thereby masking the charge of the peptide of step (a) and forming a peptide-based prodrug, which may resist protonation until after penetrating a blood stream.

An illustrative example of such modification is presented in scheme A.

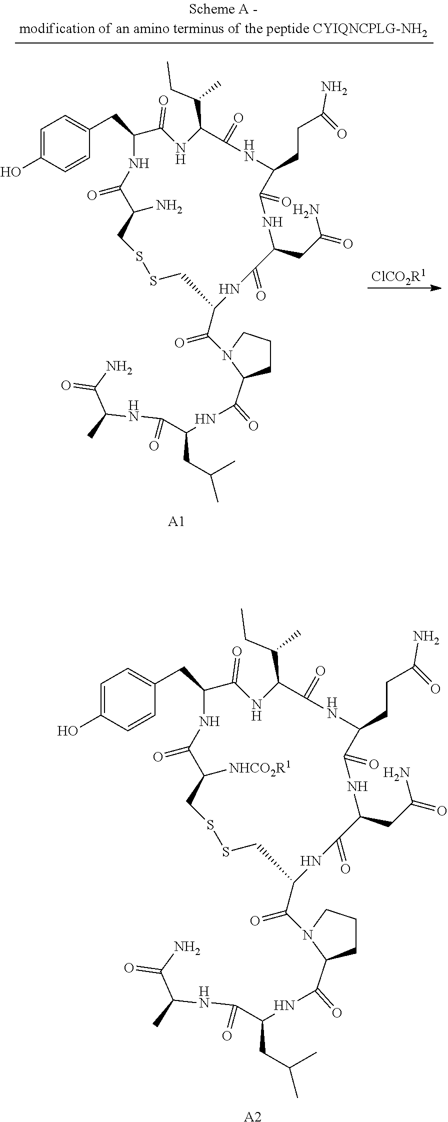

As seen in Scheme A, compound A1, which is the neuropeptide oxytocin of the sequence CYIQNCPLG-NH₂ (SEQ ID NO: 31), is reacted with a primary alkyl chloroformate to form prodrug A2 (SEQ ID NO: 32). As prodrug A2 is both more lipophilic than peptide A1 and is uncharged in physiological pH, it is contemplated that prodrug A2 would have better permeability into cells compared to peptide A1. It is further contemplated that in the blood stream, prodrug A2 would undergo an enzymatic reaction, e.g. with an esterase to form peptide A1 in the blood stream, where it is capable of executing its pharmacological effect (see, for example Scheme B). In some embodiments, R¹ is n-C₁₄H₂₉ (myristyl). In some embodiments, the peptide is oxytocin and R¹ is myristyl.

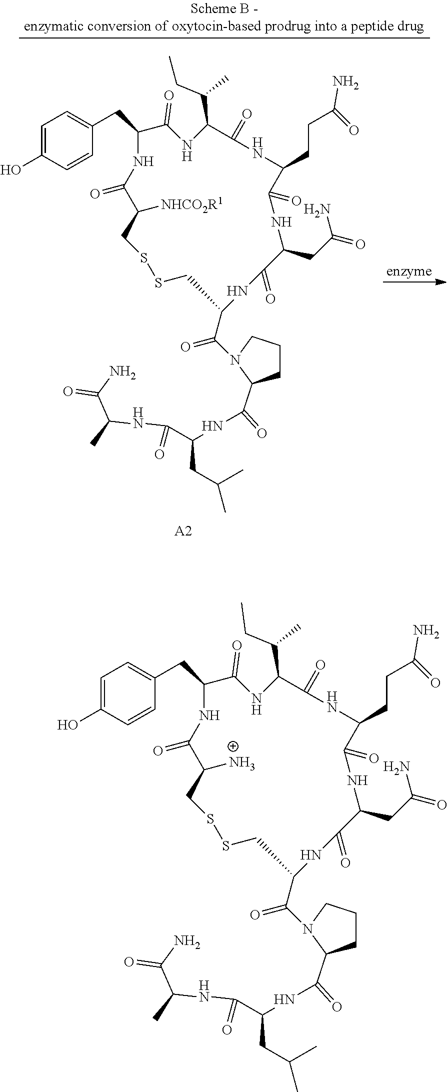

In some embodiments and as can be understood, the NH₂ group of the starting peptide's amino terminus may not be the sole basic nitrogen in the starting peptide. Rather, the starting peptide may include such amino acid residues having a nucleophilic nitrogen in its side chain, such as histidine, tryptophan and/or lysine. When such side chain(s) appear in the starting peptide (i.e. the peptide of step (a)), similar chemical transformation(s) may occur on their corresponding nucleophilic nitrogen atom, thereby reducing their basicity and tendency to form a positive charge before reaching the blood stream. Further, similar chemical transformation(s) add to the number of carbamate groups in the prodrug, thereby increasing its lipophilicity and blood stream permeability.

In some embodiments the peptide of step (a) comprises at least one amino acid residue comprising a side chain, which comprises NH and/or NH₂ moiety. In some embodiments the peptide of step (a) comprises at least one amino acid residue selected from the group consisting of histidine, lysine, tryptophan and combinations thereof. Each possibility represents a separate embodiment of the invention.

In some embodiments the peptide-based prodrug comprises at least one amino acid residue comprising a side chain, which comprises NR²CO₂R¹. In some embodiments the peptide-based prodrug comprises at least one carbamate moiety having a formula selected from the group consisting of:

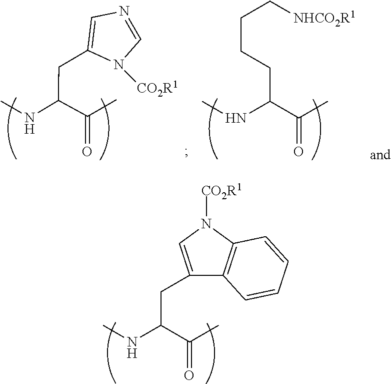

In some embodiments the peptide-based prodrug comprises at least one carbamate moiety having the formula

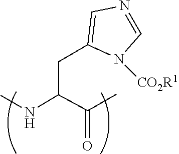

In some embodiments the peptide-based prodrug comprises at least one carbamate moiety having the formula

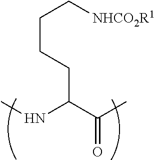

In some embodiments the peptide-based prodrug comprises at least one carbamate moiety having the formula

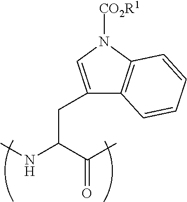

An illustrative example of such modification is presented in scheme C.

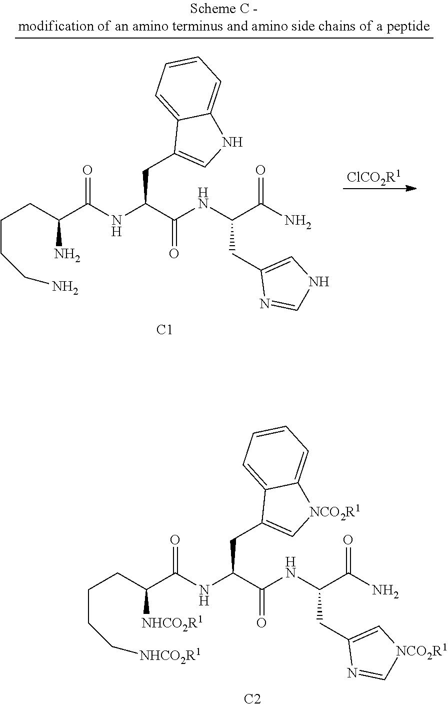

As seen in Scheme C, compound C1, which is the peptide Lys-Trp-His-NH₂, is reacted with a primary alkyl chloroformate to form prodrug C2. As prodrug C2 is both more lipophilic than peptide A1 and it is uncharged in physiological pH, it is contemplated that prodrug C2 would have better permeability into the blood stream compared to peptide C1. It is further contemplated that in the blood stream, prodrug C2 would undergo an enzymatic reaction, e.g. with an esterase to form peptide C1 in the blood stream, where it is capable of executing its pharmacological effect (see, for example Scheme D).

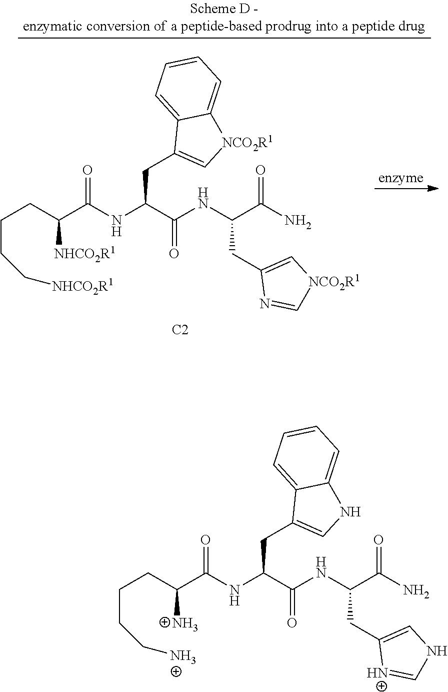

In some embodiments and generally, the transformations presented above (Schemes A and C) are relating to converting amines to carbamates. In some embodiments as the starting amine-containing peptides are basic, they may be protonated under gastrointestinal/physiological pH and thus, the transformations entail inhibiting the prodrug from acquiring positive charge.

In some embodiments the peptide-based prodrug is devoid of positively charged nitrogen atoms. In some embodiments the peptide-based prodrug is devoid of electrically charged nitrogen atoms. In some embodiments the peptide-based prodrug is having a net neutral charge. In some embodiments the peptide-based prodrug is devoid of positively charged atoms. In some embodiments the peptide-based prodrug is devoid of charged atoms. In some embodiments the peptide-based prodrug is devoid of positively charged nitrogen atoms at physiological pH. In some embodiments the peptide-based prodrug is devoid of electrically charged nitrogen atoms at physiological pH. In some embodiments the peptide-based prodrug is having a net neutral charge at physiological pH. In some embodiments the peptide-based prodrug is devoid of positively charged atoms at physiological pH. In some embodiments the peptide-based prodrug is devoid of charged atoms at physiological pH. It is to be understood that physiological pH is around 7.3. In some embodiments the peptide-based prodrug is devoid of positively charged nitrogen atoms at gastrointestinal pH. In some embodiments the peptide-based prodrug is devoid of electrically charged nitrogen atoms at gastrointestinal pH. In some embodiments the peptide-based prodrug is having a net neutral charge at gastrointestinal pH. In some embodiments the peptide-based prodrug is devoid of positively charged atoms at gastrointestinal pH. In some embodiments the peptide-based prodrug is devoid of charged atoms at gastrointestinal pH.

In some embodiment and as understood by a person skilled in the art, the reaction of step (b) may be facilitated in the presence of a base. Without wishing to be bound by any theory or mechanism of action, the peptide of step (a) may include protonated nitrogen atoms. Consequently, said protonated nitrogen atoms may show very low nucleophilicity and tendency to react with the alkyl chloroformate. As a result, an added base may deprotonate the protonated nitrogen atoms of the starting peptide and facilitate the reaction.

In some embodiment step (b) is preformed in the presence of a base. In some embodiment step (b) further comprises adding a base to the mixture of step (b).

In some embodiment the base is selected from an amine, a carbonate, a phosphate, a bicarbonate a hydroxide or a combination thereof. In some embodiment the base is an amine. In some embodiment the base is trimethylamine and/or N,N-diisopropylethylamine. In some embodiment the base is triethylamine. In some embodiment the base is N,N-diisopropylethylamine.

In some embodiment step (b) is performed in a solvent selected from the group consisting of acetonitrile, dimethyl formamide, dimethyl acetamide, dimethyl sulfoxide, ethanol, methanol and mixtures thereof. In some embodiment the solvent is acetonitrile.

In some embodiments, although the transformations presented above entail inhibiting the prodrug from acquiring positive charge, it may also be desirable to inhibit negative charge(s) in peptides as well, for enhancing the blood stream permeability of the prodrugs. In some embodiments and typically, negative charges on peptides may be derived from carboxylate groups, such as the starting peptide's carboxylic terminus, glutamic acid side chain(s) and/or aspartic acid side chain(s). It was found that such negative charges may be masked using SOCl₂ mediated esterification. It was further found that upon administration of the esterified prodrug, the ester groups may remain intact until reaching the target therapeutic location; while in this location they undergo enzymatic de-esterification to their former state.

In some embodiments the peptide of step (a) comprises at least COOH moiety. In some embodiments the peptide of step (a) comprises at least one amino acid residue comprising a side chain, which comprises COOH moiety. In some embodiments the peptide of step (a) comprises at least one amino acid residue selected from the group consisting of aspartic acid, glutamic acid and combinations thereof. In some embodiments the peptide of step (a) comprises at least one aspartic acid residue. In some embodiments the peptide of step (a) comprises peptide comprises at least one glutamic acid residue.

It is to be understood that the esterification may occur before or after the reaction of the starting peptide with the alkyl chloroformate.

In some embodiments the process further comprises a step of esterifying the peptide of step (a). In some embodiments the process further comprises a step of esterifying the prodrug of step (b). In some embodiments the process further comprises a step of reacting the peptide of step (a) or the peptide-based prodrug of step (b) with an alcohol in the presence of an esterification reagent. In some embodiments the esterification reagent is selected from the group consisting of thionyl chloride, oxalyl chloride, phosphorous pentachloride, phosphorous trichloride, phosphoryl chloride, phosgene, diethyl azodicarboxylate (DEAD), diisopropyl azodicarboxylate (DIAD), N,N′-diisopropylcarbodiimide (DIPC), N,N′-dicyclohexylcarbodiimide (DCC) and di-tert-butyl dicarbonate. Each possibility represents a separate embodiment. In some embodiments the esterification reagent is thionyl chloride.

In some embodiments the process further comprises a step of reacting the peptide-based prodrug with an alcohol in the presence of thionyl chloride. In some embodiments the process further comprises step (c) of reacting the peptide-based prodrug with an alcohol in the presence of an esterification reagent. In some embodiments step (a) further comprises reacting the peptide with an alcohol in the presence of an esterification reagent.

In some embodiments there is provided a process for preparing a peptide-based prodrug, the process comprising:
(a) providing a peptide precursor;
(b) coupling said peptide precursor with a modified amino acid having a formula selected from the group consisting of:

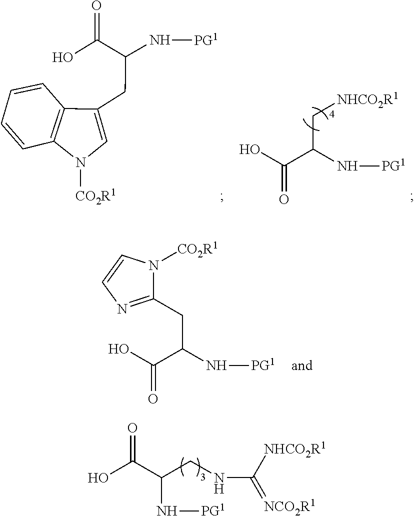

wherein
R¹ is a primary alkyl,
PG¹ is a base-labile protecting group;
wherein the peptide precursor is selected from the group consisting of: an amino acid, a peptide and a solid phase resin.
(c) removing said base-labile protecting group PG from the product of step (b) under basic conditions; and
(d) optionally coupling at least one additional amino acid;
   thereby forming the peptide-based prodrug.

In some embodiments, there is provided a peptide-based prodrug, prepared by a process comprising:
(a) providing a peptide precursor;
(b) coupling said peptide precursor with a modified amino acid having a formula selected from the group consisting of:

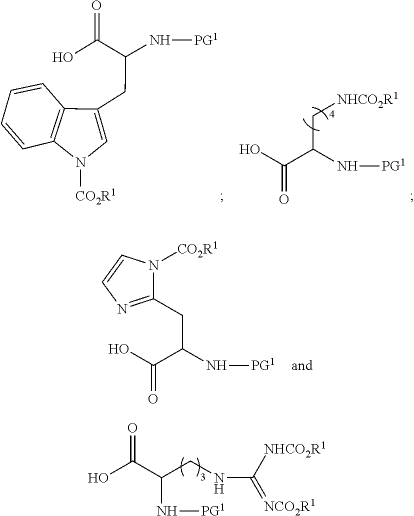

wherein
R¹ is a primary alkyl,
PG¹ is a base-labile protecting group;
wherein the peptide precursor is selected from the group consisting of: an amino acid, a peptide and a solid phase resin.
(c) removing said base-labile protecting group PG¹ from the product of step (b) under basic conditions; and
(d) optionally coupling at least one additional amino acid;
   thereby forming the peptide-based prodrug.

In some embodiments and generally, peptides prepared by the process above are characterized by having a lipophilic CO₂R¹ fragment(s). Specifically, the modified amino acid(s), which act as building block(s), provide lipophilic carbamate fragment(s) to the prodrug.

Illustrative examples of preparing the modified amino acid building blocks are presented in Schemes E-I:

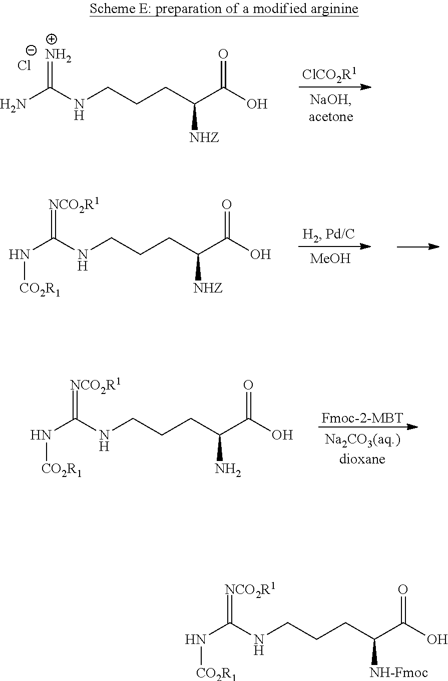

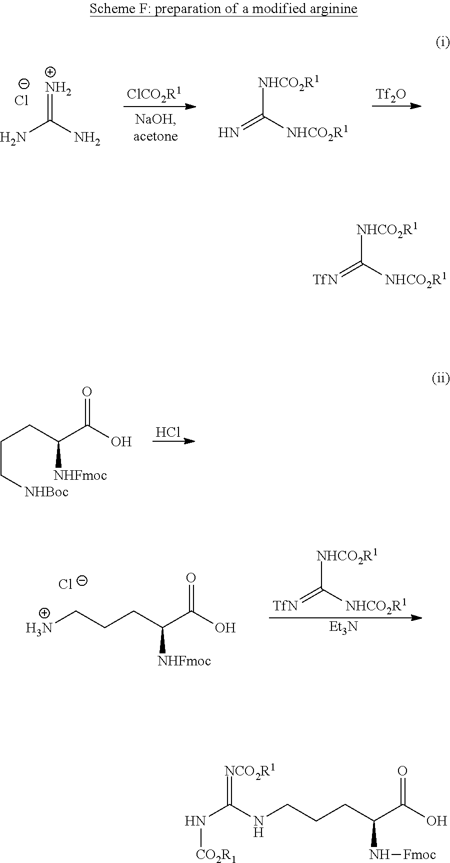

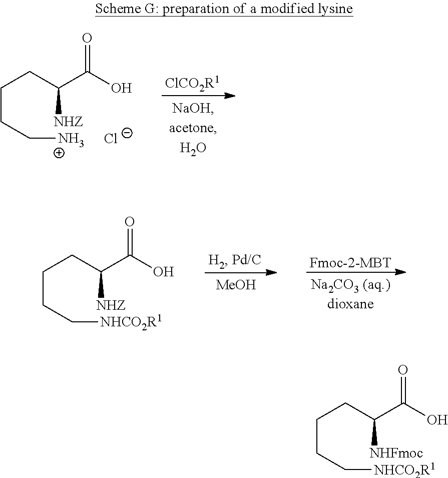

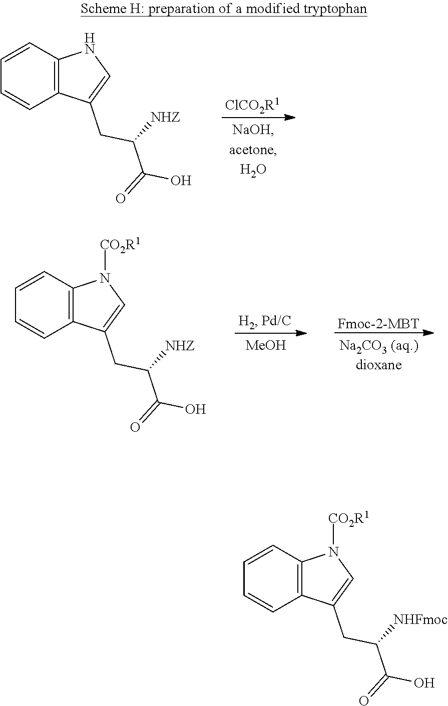

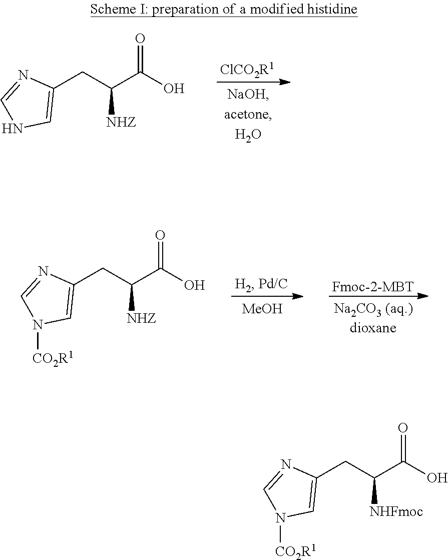

As used herein, “Z” symbolizes carboxybenzyl; “Fmoc-2-MBT” symbolizes Fmoc-2-Mercaptobenzothiazole; “Fmoc” symbolizes fluorenylmethyloxycarbonyl; “Tf₂O” symbolizes trifluoromethanesulfonic anhydride; “Tf” symbolizes trifluoromethanesulfonyl; and “Boc” symbolizes tert-butyloxycarbonyl.

In some embodiments R′ is a primary alkyl group as defined hereinabove. In some embodiments R² is as defined hereinabove. Appropriately, the alkyl chloroformate in Schemes E-I may be having an alkyl as described above according to some embodiments. In some embodiments, the peptide-based prodrug comprises said alkyl group. Specifically, in some embodiments, the peptide-based prodrug comprises at least one NR²CO₂R¹ moiety.

In some embodiments step (d) comprises coupling at least one additional amino acid. In some embodiments step (d) comprises coupling a plurality of additional amino acid. In some embodiments the additional amino acid(s) is a protected amino acid. In some embodiments the additional amino acid(s) is an amino protected amino acid. In some embodiments the amino protected amino acid is protected by a base-labile protecting group.

The term “plurality” refers to at least two items.

In some embodiments and as understood the process above refers to incorporation of modified amino acid building block(s) to the skeleton of a peptide-based prodrug. Specifically, it refers to incorporation of modified arginine, tryptophan, lysine and/or histidine building block(s) to the skeleton of the peptide-based prodrug. The incorporation may be performed during the peptide synthesis, and thus it may be set up to the stage, when an arginine, tryptophan, lysine and/or histidine is to be incorporated to form the desired peptide. In some embodiments when arginine, tryptophan, lysine and/or histidine is to be inserted last (i.e. prodrugs of a peptide having terminal residue of arginine, tryptophan, lysine or histidine), the coupling of additional amino in acid step (d) may be unneeded. On the other hand, in embodiments when arginine, tryptophan, lysine and/or histidine is to be inserted in other positions in the peptide sequence, the coupling of additional amino in acid step (d) may be required.

In some embodiments the peptide-based prodrug is a cyclic peptide-based prodrug. In some embodiments the process further comprises a step of cyclizing the peptide-based prodrug to form a cyclic peptide-based prodrug.

In some embodiments the cyclic peptide-based prodrug has a backbone to backbone intramolecular bond. In some embodiments the cyclic peptide-based prodrug has a backbone to backbone intramolecular bond between the N-terminus and the C-terminus of the peptide. In some embodiments the cyclic peptide based prodrug has a backbone to side-chain intramolecular bond. In some embodiments the cyclic peptide-based prodrug has a side-chain to side-chain intramolecular bond. In some embodiments the cyclic peptide-based prodrug has a side-chain to side-chain intramolecular disulfide bond between two cysteine side chain residues. In some embodiments the cyclic peptide-based prodrug does not include an amino terminus.

In some embodiments the cyclic peptide is somatostatin or a somatostatin analog.

In some embodiments the modified amino acid of step (b) is having a formula selected from the group consisting of:

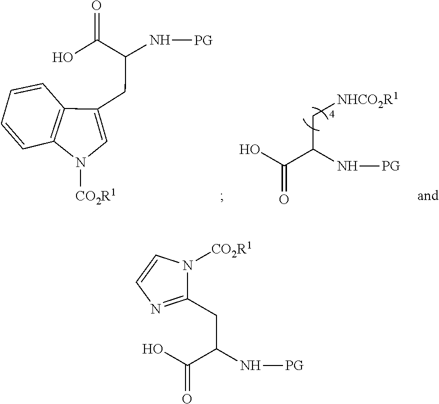

In some embodiments the modified amino acid is having a formula selected from the group consisting of:

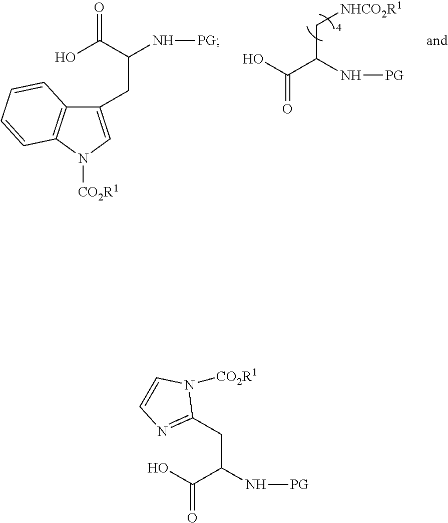

In some embodiments the modified amino acid is having the formula

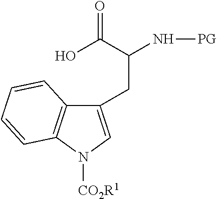

In some embodiments the modified amino acid is having the formula

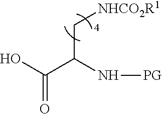

In some embodiments the modified amino acid is having the formula

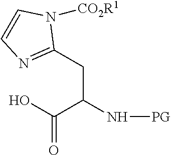

In some embodiments the modified amino acid is having the formula:

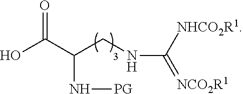

The term “solid phase resin”, “solid support resin” and “solid support” as used herein are interchangeable and intended to mean an insoluble polymeric matrix whereupon a molecule, e.g. a ligand in the form of a polypeptide, can be synthesized or coupled with or without a linker or spacer in-between. solid support resins are typically used in peptide synthesis. These polymers are generally employed in the form of beads. Polymer resins preferred for peptide synthesis are polystyrenes, polyacrylamides and the like, specifically copolymers of styrene and divinylbenzene. Prior to the coupling with the first amino acid, the solid support resin contains surface functionality or can be derivatized to contain surface functionality which can interact with an amine group of an amino acid (or peptide) so as to attach the amino acid (or peptide) to the support directly or indirectly through the amine group of the peptide. Solid phase resin, as used herein is not limited to the parent commercial derivatized resins, in their form prior the first coupling of amino acid or peptide. Rather, after the first coupling of amino acid and during the peptide synthesis, while the resin is coupled to a growing peptide, the resin is still considered a solid phase resin. In some embodiments the solid phase resin is coupled to at least one amino acid. In some embodiments the solid phase resin is not coupled to amino acids.

The term “peptide precursor”, as used herein refers to chemical compounds, which are used in the preparation of peptides. The term includes, but not limited to amino acids, peptides, peptides hybrids, solid phase resins not coupled to amino acids, and solid phase resins coupled to amino acid(s).

In some embodiments the peptide precursor comprises a terminal primary amino group. In some embodiments the peptide precursor is selected from the group consisting of: an amino acid, a peptide and a solid phase resin. In some embodiments the peptide precursor is a solid phase resin. In some embodiments the peptide precursor is a solid phase resin not coupled to amino acids. In some embodiments the peptide precursor is a solid phase resin coupled to at least one amino acid. In some embodiments the peptide precursor is a peptide. In some embodiments the peptide precursor is an amino acid. In some embodiments the peptide precursor is a solid phase resin having at least one amino acid residue.

As used herein, “FMOC” symbolizes fluorenylmethyloxycarbonyl, “DIC” symbolizes diisopropylcarbodiimide; “DMF” symbolizes dimethylformamide; “TBAF” refers to tetra-n-butylammonium fluoride; and “DCC” refers to dicyclohexylcarbodiimide.

An illustrative example of the process of producing the peptide-based prodrug using arginine and a solid phase resin is presented in scheme J:

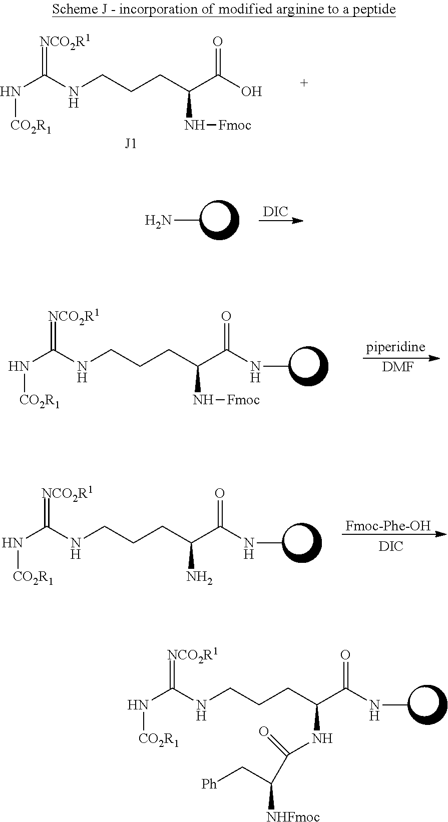

As seen in Scheme J, compound J1, which is arginine modified by a CO₂R¹ group and protected with Fmoc, is reacted with a solid phase resin having a free unprotected NH₂ group under standard coupling conditions. Thereafter, the product resin is coupled with phenylalanine as a part of peptide elongation to form a modified dipeptide bound to a resin, which may be further elongated or removed from the resin.

An illustrative example of the process of producing the peptide-based prodrug using lysine and a solid phase resin is presented in scheme K:

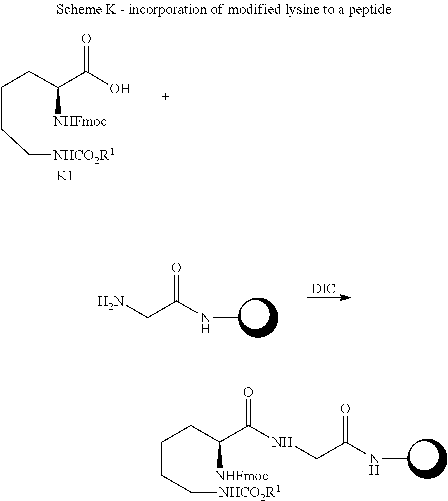

As seen in Scheme K, compound K1, which is lysine modified by a CO₂R¹ group and protected with Fmoc, is reacted with a solid phase resin coupled to glycine under standard coupling conditions. This forms a modified dipeptide bound to a resin, which may be further elongated or removed from the resin.

An illustrative example of the process of producing the peptide-based prodrug using tryptophan and a solid phase resin coupled to an amino acid is presented in scheme L:

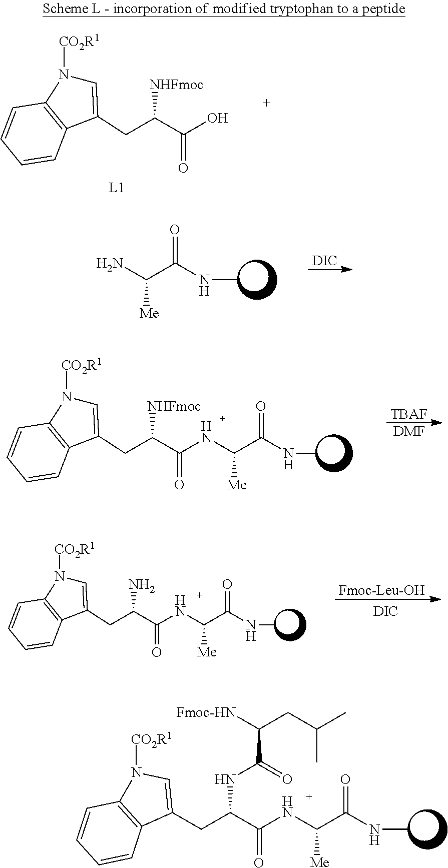

As seen in Scheme L, compound L1, which is tryptophan modified by a CO₂R¹ group and protected with Fmoc, is reacted with a solid phase resin coupled to alanine under standard coupling conditions. Thereafter, the product is coupled with leucine as a part of peptide elongation to form a modified tripeptide bound to a resin, which may be further elongated or removed from the resin.

An illustrative example of the process of producing the peptide-based prodrug using histidine and an amino acid is presented in scheme M:

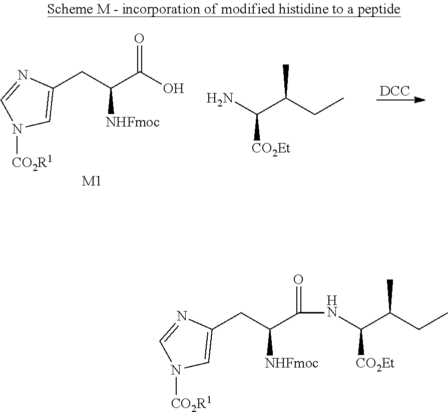

As seen in Scheme M, compound Ml, which is histidine modified by a CO₂R¹ group and protected with Fmoc, is reacted with isoleucine ethyl ester under standard coupling conditions. This forms a modified dipeptide, which may be further elongated deprotected.

It is contemplated that in the blood stream, prodrugs prepared according to the above processes would undergo an enzymatic reaction, e.g. with an esterase to form the corresponding peptides in the blood stream, where they are capable of executing their pharmacological effect (see, for example Schemes D and N). Scheme N shows enzymatic conversion of a peptide-based prodrug N1 (SEQ ID NO: 33) into a peptide drug N2 (vasopressin, SEQ ID NO: 34).

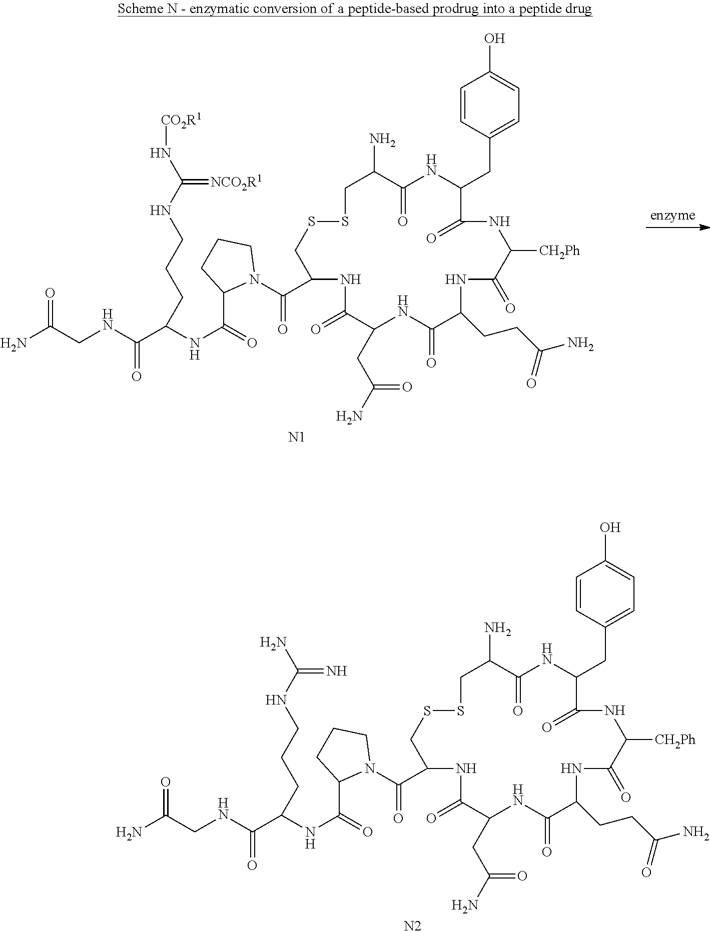

In some embodiments the process further comprises step (e) of removing the peptide-based prodrug from the solid phase resin. In some embodiments the process further comprises a step of removing the peptide-based prodrug from the solid phase resin.

In some embodiments the PG¹ is a base-labile protecting group. The term “base-labile protecting group” refers to a protecting group that can be removed by treatment with an aqueous or non-aqueous base. In some embodiments the PG¹ is fluorenylmethyloxycarbonyl (Fmoc).

In some embodiments the coupling of step (b) comprises contacting the peptide precursor and the modified amino acid in the presence of an amino acid coupling agent. In some embodiments the coupling of step (b) comprises contacting the peptide precursor and the modified amino acid in the presence of a coupling agent selected from a carbodiimide, 1-[Bis(dimethylamino)methylene]-1H-1,2,3-triazolo[4,5-b]pyridinium 3-oxid hexafluorophosphate), 1-Hydroxy-7-azabenzotriazole and combinations thereof. In some embodiments the carbodiimide is dicyclohexyl carbodiimide or diisopropyl carbodiimide. Each possibility represents a separate embodiment.

In some embodiments the peptide-based prodrug comprises at least one carbamate moiety having the formula —NR²CO₂R¹. In some embodiments the peptide-based prodrug comprises at least one amino acid residue comprising a side chain, which comprises NCO₂R¹ and/or NHCO₂R¹ moiety. In some embodiments the peptide-based prodrug comprises at least one amino acid residue comprising a side chain, which comprises —NR²CO₂R¹ moiety. In some embodiments the NR²CO₂R¹ moiety has a formula selected from the group consisting of:

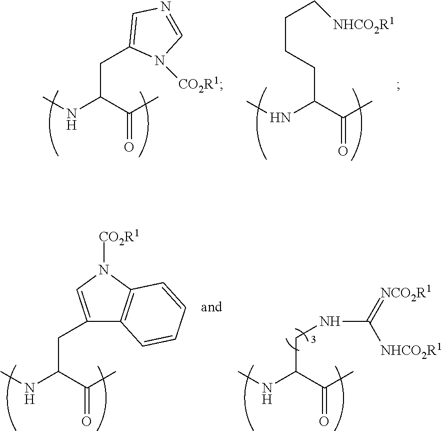

In some embodiments the peptide-based prodrug comprises at least one carbamate moiety having a formula selected from the group consisting of:

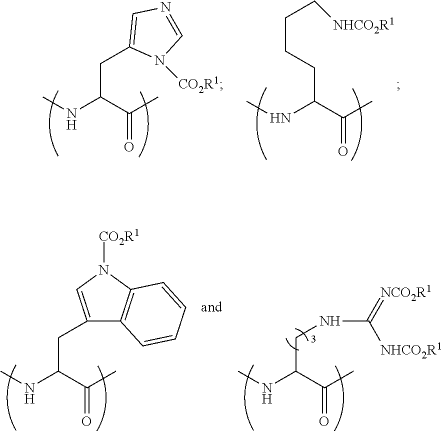

In some embodiments the peptide-based prodrug comprises at least one carbamate moiety having a formula selected from the group consisting of:

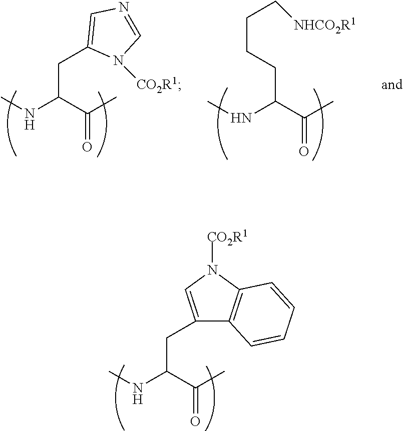

In some embodiments the peptide-based prodrug comprises at least one carbamate moiety having the formula:

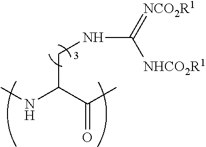

In some embodiments the peptide-based prodrug comprises at least one carbamate moiety having the formula:

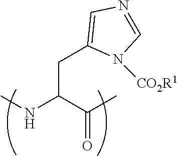

In some embodiments the peptide-based prodrug comprises at least one carbamate moiety having the formula:

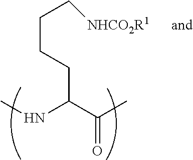

In some embodiments the peptide-based prodrug comprises at least one carbamate moiety having the formula:

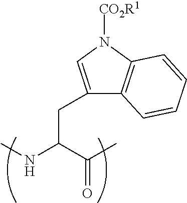

In some embodiments and generally, the transformations presented above (Schemes J, K, L and M) are relating to converting amines to carbamates. In some embodiments as the starting amine-containing peptides are basic, they may be protonated under gastrointestinal/physiological pH and thus, the transformations entail inhibiting the prodrug from acquiring positive charge.

In some embodiments the peptide-based prodrug is devoid of positively charged nitrogen atoms. In some embodiments the peptide-based prodrug is devoid of electrically charged nitrogen atoms. In some embodiments the peptide-based prodrug is having a net neutral charge. In some embodiments the peptide-based prodrug is devoid of positively charged atoms. In some embodiments the peptide-based prodrug is devoid of charged atoms. In some embodiments the peptide-based prodrug is devoid of positively charged nitrogen atoms at physiological pH. In some embodiments the peptide-based prodrug is devoid of electrically charged nitrogen atoms at physiological pH. In some embodiments the peptide-based prodrug is having a net neutral charge at physiological pH. In some embodiments the peptide-based prodrug is devoid of positively charged atoms at physiological pH. In some embodiments the peptide-based prodrug is devoid of charged atoms at physiological pH. In some embodiments the peptide-based prodrug is devoid of positively charged nitrogen atoms at gastrointestinal pH. In some embodiments the peptide-based prodrug is devoid of electrically charged nitrogen atoms at gastrointestinal pH. In some embodiments the peptide-based prodrug is having a net neutral charge at gastrointestinal pH. In some embodiments the peptide-based prodrug is devoid of positively charged atoms at gastrointestinal pH. In some embodiments the peptide-based prodrug is devoid of charged atoms at gastrointestinal pH.

In some embodiments and as mentioned above, it may also be desirable to inhibit negative charge(s) in peptides for enhancing the blood stream permeability of the prodrugs.

In some embodiments the peptide precursor of step (a) and/or the at least one additional amino acid comprises at least COOH moiety. In some embodiments the peptide precursor of step (a) and/or the at least one additional amino acid comprises at least one amino acid residue comprising a side chain, which comprises COOH moiety. In some embodiments the peptide precursor of step (a) and/or the at least one additional amino acid comprises at least one amino acid residue selected from the group consisting of aspartic acid, glutamic acid and combinations thereof.

It is to be understood that the esterification may occur before or after the reaction of the starting peptide precursor with the modified amino acid.

In some embodiments the process further comprises a step of esterifying the COOH moiety. In some embodiments the process further comprises a step of esterifying a COOH containing compound selected from the peptide precursor, the product of step (c) or the product of step (d). In some embodiments the process further comprises a step of esterifying the product of step (c) or (d). In some embodiments the process further comprises a step of esterifying the product of step (d). In some embodiments the process further comprises a step of esterifying the prodrug of step (d). In some embodiments the esterification comprises reacting the COOH containing compound with an alcohol in the presence of an esterification reagent. In some embodiments the esterification reagent is selected from the group consisting of thionyl chloride, oxalyl chloride, phosphorous pentachloride, phosphorous trichloride, phosphoryl chloride, phosgene, diethyl azodicarboxylate (DEAD), diisopropyl azodicarboxylate (DIAD), N,N′-diisopropylcarbodiimide (DIPC), N,N′-dicyclohexylcarbodiimide (DCC) and di-tert-butyl dicarbonate. Each possibility represents a separate embodiment. In some embodiments the esterification reagent is thionyl chloride. In some embodiments the process further comprises a step of reacting the peptide-based prodrug with an alcohol in the presence of thionyl chloride. In some embodiments the process further comprises step (e) of reacting the peptide-based prodrug with an alcohol in the presence of thionyl chloride.

In some embodiments the peptide-based comprises at least one COOR³ moiety. In some embodiments R³ is other than hydrogen or a metal. In some embodiments R³ is an alkyl group. In some embodiments R³ is an alkyl group selected from methyl, ethyl and isopropyl. In some embodiments R³ is an alkyl group selected from methyl and ethyl. In some embodiments R³ is ethyl.

In some embodiments the COOR³ moiety is a part of an amino acid side chain selected from aspartic acid and glutamic acid.

In some embodiments the peptide-based prodrug comprises no more than a single COOH group. In some embodiments the peptide-based prodrug is devoid of COOH groups.

In some embodiments there is provided a process for preparing a peptide-based prodrug, the process comprises
(a) providing a peptide precursor;
(b) coupling said peptide precursor with a protected amino acid having a formula selected from the group consisting of:

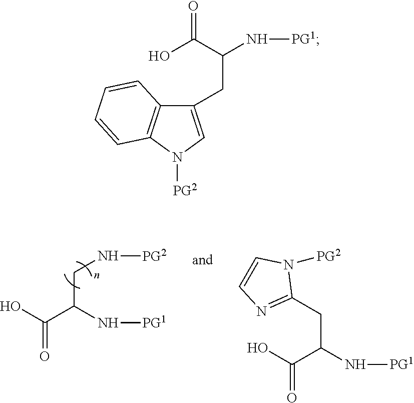

wherein
PG¹ is a base-labile protecting group;
PG² is an acid-labile protecting group;
n is 3 or 4;
wherein the peptide precursor is selected from the group consisting of: an amino acid, a peptide and a solid phase resin;
(c) removing said acid-labile protecting group PG² from the product of step (b) under acidic conditions;
(d) reacting the product of step (c) with a compound having a formula selected from

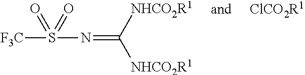

wherein R¹ is a primary alkyl;
(e) removing said base-labile protecting group PG¹ under basic conditions; and
(f) optionally coupling at least one additional amino acid;
   thereby forming the peptide-based prodrug.

In some embodiments, there is provided a peptide-based prodrug, prepared by a process comprising:
(a) providing a peptide precursor;
(b) coupling said peptide precursor with a protected amino acid having a formula selected from the group consisting of:

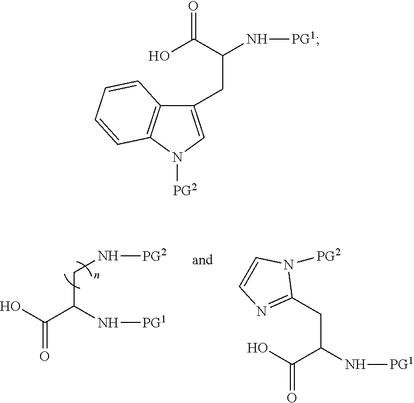

wherein
PG¹ is a base-labile protecting group;
PG² is an acid-labile protecting group;
n is 3 or 4;
wherein the peptide precursor is selected from the group consisting of: an amino acid, a peptide and a solid phase resin.
(c) removing said acid-labile protecting group PG² from the product of step (b) under acidic conditions;
(d) reacting the product of step (c) with a compound having a formula selected from

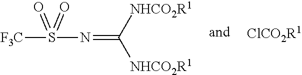

wherein R¹ is a primary alkyl;
(e) removing said base-labile protecting group PG¹ under basic conditions; and
(f) optionally coupling at least one additional amino acid;
   thereby forming the peptide-based prodrug.

In some embodiments and generally, peptides prepared by the process above are characterized by having a lipophilic CO₂R¹ fragment(s). Specifically, one or more amino acid residue is being modified during the process, thus providing lipophilic NR²CO₂R¹ fragment(s) to the prodrug.

In some embodiments R¹ is a primary alkyl group as defined hereinabove. In some embodiments R² is as defined hereinabove. Appropriately, the alkyl chloroformate and modified guanidine in of step (d) may be having an alkyl as described above according to some embodiments. In some embodiments, the peptide-based prodrug comprises said alkyl group. Specifically, in some embodiments, the peptide-based prodrug comprises at least one carbamate moiety.

In some embodiments step (f) comprises coupling one additional amino acid. In some embodiments step (f) comprises coupling a plurality of additional amino acids.

In some embodiments and as understood the process above refers to modification(s) of amino acid residue(s) within the skeleton of a peptide-based prodrug. Specifically, it refers to formation of modified arginine, tryptophan, lysine and/or histidine residue(s) in the skeleton of the peptide-based prodrug. The modification, which is accomplished in step (d) may be performed during different stages of the peptide synthesis, depending e.g. on the number of modified amino acids required and on the stage, when they are to be incorporated to form the desired peptide. In some embodiments step (b) further comprises coupling at least one additional amino acid after the coupling of the protected amino acid defined in step (b).

In some embodiments the peptide-based prodrug is a cyclic peptide based prodrug. In some embodiments the process further comprises a step of cyclizing the peptide-based prodrug to form a cyclic peptide-based prodrug.

In some embodiments the cyclic peptide-based prodrug has a backbone to backbone intramolecular bond. In some embodiments the cyclic peptide-based prodrug has a backbone to backbone intramolecular bond between the N-terminus and the C-terminus of the peptide. In some embodiments the cyclic peptide based prodrug has a backbone to side-chain intramolecular bond. In some embodiments the cyclic peptide based prodrug has a side-chain to side-chain intramolecular bond. In some embodiments the cyclic peptide-based prodrug has a side-chain to side-chain intramolecular disulfide bond between two cysteine side chain residues. In some embodiments the cyclic peptide-based prodrug does not include an amino terminus.

In some embodiments the cyclic peptide is somatostatin or a somatostatin analog.

In some embodiments the protected amino acid is having the formula

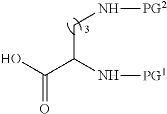

In some embodiments the reaction of step (d) is with the compound having the formula

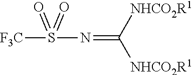

In some embodiments the protected amino acid is having the formula

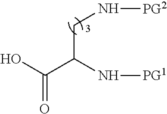

and the reaction of step (d) is with the compound having the formula

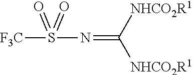

In some embodiments the peptide-based prodrug comprises at least one carbamate moiety having the formula:

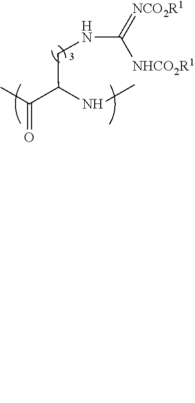

As used herein, “Mtt” symbolizes 4-Methyltrityl; “NMP” symbolizes N-methyl pyrrolidinone; “HATU” symbolizes 1-[Bis(dimethylamino)methylene]-1H-1,2,3-triazolo[4,5-b]pyridinium 3-oxid hexafluorophosphate); “TIPS” symbolizes triisopropylsilane; and “HOAt” symbolizes 1-Hydroxy-7-azabenzotriazole.

An illustrative example of the process of producing the peptide-based prodrug using ornithine and a solid phase resin is presented in scheme O:

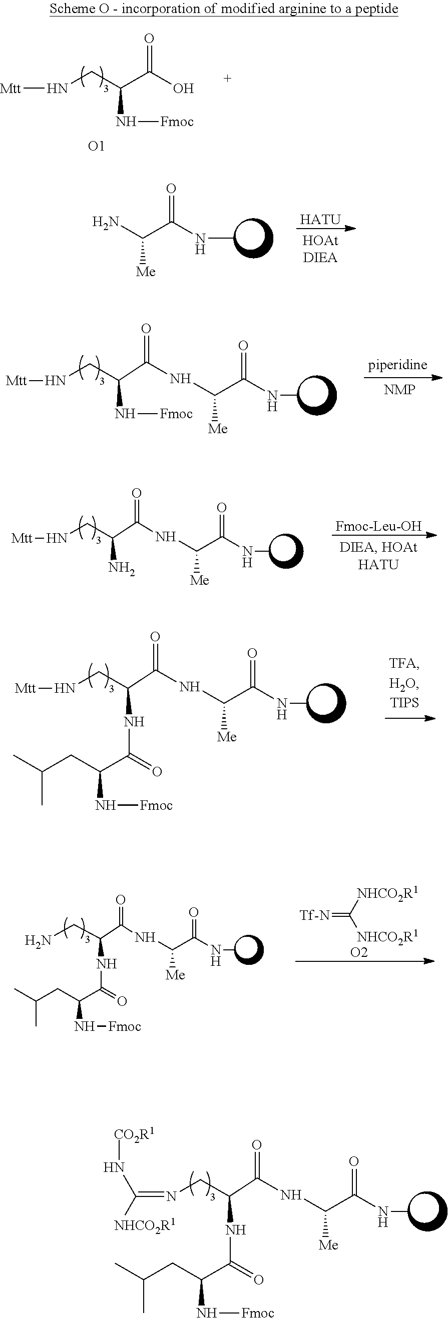

As seen in Scheme O, compound O1, which is ornithine modified by an acid labile Mtt group at the side chain and by a base-labile Fmoc group at the alpha nitrogen, is reacted with a solid phase resin coupled to alanine under standard coupling conditions. Thereafter, the base-labile Fmoc group is removed and the product is coupled with leucine as a part of peptide elongation. Then, the acid-labile Mtt group is removed under acidic conditions and the product is reacted with modified guanidine O2 to form a modified tripeptide bound to a resin, which may be further elongated or removed from the resin.

In some embodiments, the reaction sequence may be changed. An illustrative example of a similar process using ornithine and a solid phase resin is presented in scheme P:

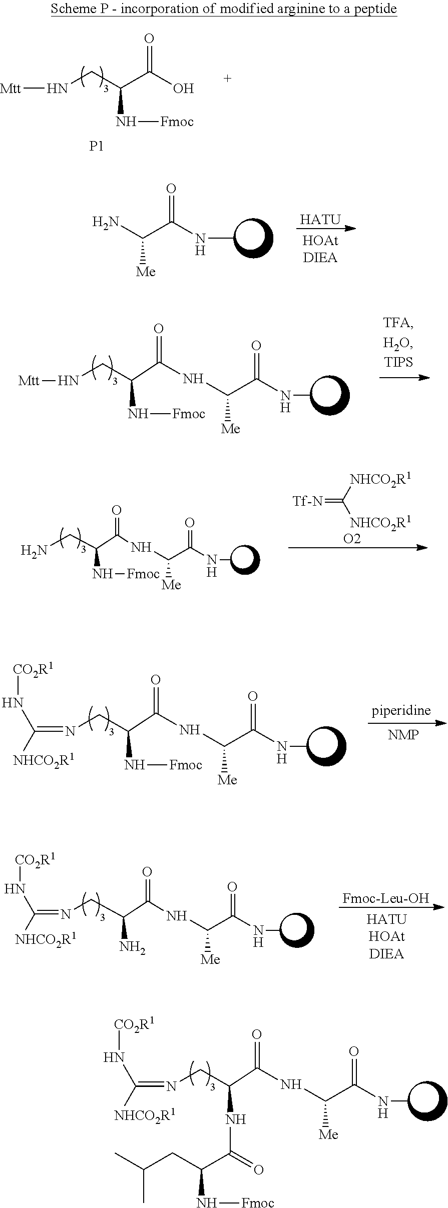

As seen in Scheme P, compound P1, which is ornithine modified by an acid labile Mtt group at the side chain and by a base-labile Fmoc group at the alpha nitrogen, is reacted with a solid phase resin coupled to alanine under standard coupling conditions. Then, the acid-labile Mtt group is removed under acidic conditions and the product is reacted with modified guanidine O2 to form a modified dipeptide bound to a resin. Thereafter, the base-labile Fmoc group is removed and the product is coupled with leucine as a part of peptide elongation to form a modified tripeptide bound to a resin, which may be further elongated or removed from the resin.

The modified guanidine O2 may be prepared as illustrated in Scheme Q:

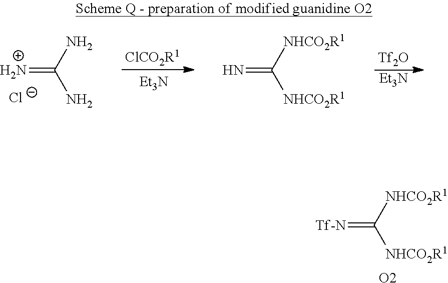

In some embodiments the protected amino acid is having a formula selected from the group consisting of:

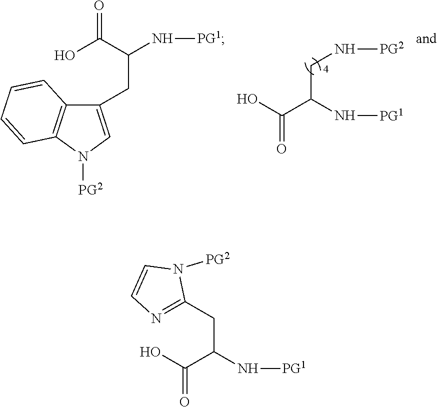

In some embodiments the protected amino acid is having the formula:

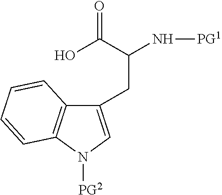

In some embodiments the protected amino acid is having the formula:

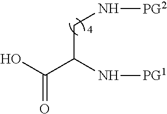

In some embodiments the protected amino acid is having the formula:

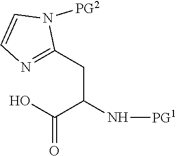

In some embodiments the reaction of step (d) is with a compound having the formula ClCO₂R¹.

In some embodiments the peptide-based prodrug comprises at least one carbamate moiety having a formula selected from the group consisting of:

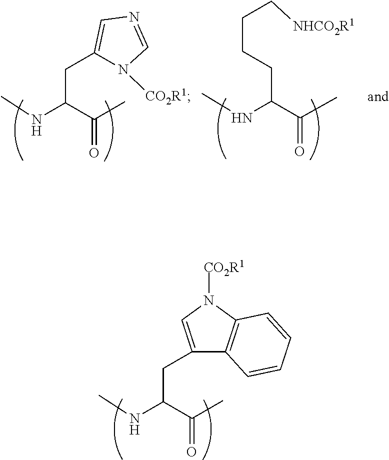

In some embodiments the peptide-based prodrug comprises at least one carbamate moiety having the formula:

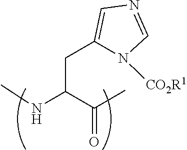

In some embodiments the peptide-based prodrug comprises at least one carbamate moiety having the formula:

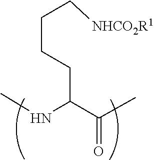

In some embodiments the peptide-based prodrug comprises at least one carbamate moiety having the formula:

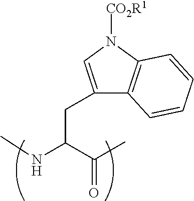

In some embodiments step (b) further comprises removing said base-labile protecting group under basic conditions; and coupling at least one additional amino acid having a second base labile protecting group, wherein step (e) comprises removing said second base-labile protecting group under basic conditions. In some embodiments step (b) further comprises removing said base-labile protecting group under basic conditions; and coupling a plurality of additional amino acids, each having a second base labile protecting group, wherein step (e) comprises removing each of said second base-labile protecting groups under basic conditions.

In some embodiments step (a) further comprises coupling at least one additional amino acid having an additional base labile protecting group and removing said additional base labile protecting group under basic conditions.

In some embodiments step (a) further comprises coupling at least one preceding amino acid having a preceding base labile protecting group and removing said base labile protecting group under basic conditions.

Illustrative examples of the processes including modifications of tryptophan, lysine and histidine are presented in Schemes R and S:

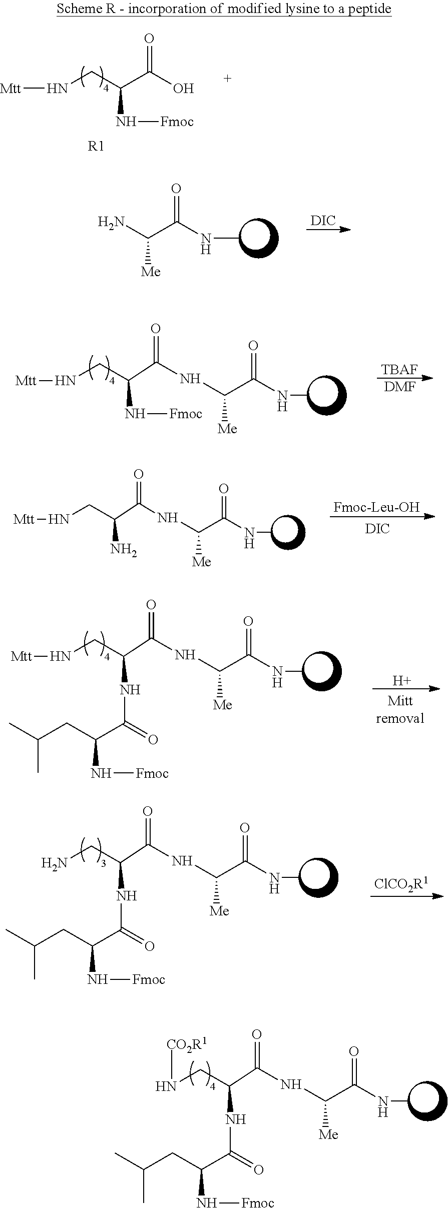

As seen in Scheme R, compound R1, which is lysine modified by an acid labile Mtt group at the side chain and by a base-labile Fmoc group at the alpha nitrogen, is reacted with a solid phase resin coupled to alanine under standard coupling conditions. Thereafter, the base-labile Fmoc group is removed and the product is coupled with leucine as a part of peptide elongation. Then, the acid-labile Mtt group is removed under acidic conditions and the product is reacted with an alkyl chloroformate to form a modified tripeptide bound to a resin, which may be further elongated or removed from the resin.

As used herein, “DIEA” symbolizes N,N-diisopropylethylamine.

In some embodiments, the reaction sequence may be changed. An illustrative example of a similar process using histidine and a solid phase resin is presented in scheme S:

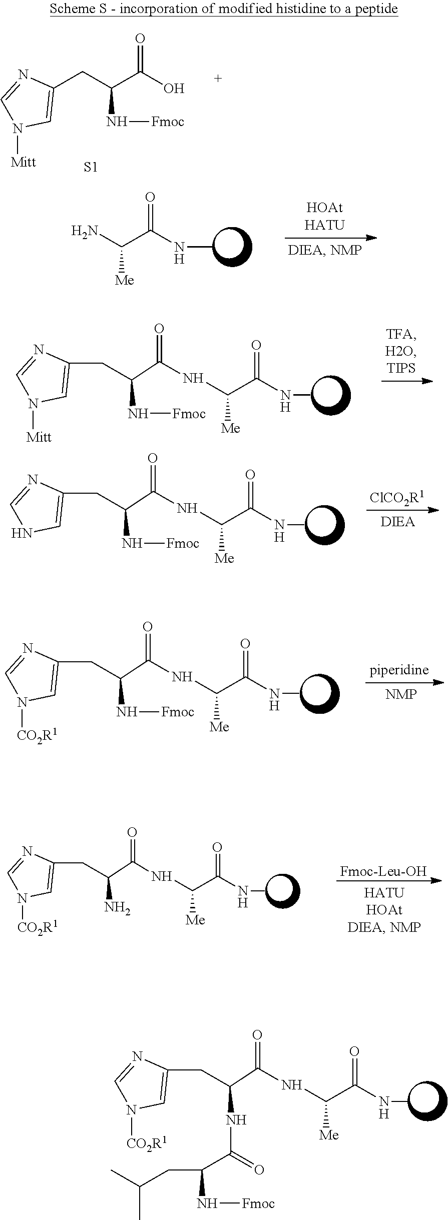

As seen in Scheme S, compound S1, which is histidine modified by an acid labile Mtt group at the side chain and by a base-labile Fmoc group at the alpha nitrogen, is reacted with a solid phase resin coupled to alanine under standard coupling conditions. Then, the acid-labile Mtt group is removed under acidic conditions and the product is reacted with an alkyl chloroformate to form a modified dipeptide bound to a resin. Thereafter, the base-labile Fmoc group is removed and the product is coupled with leucine as a part of peptide elongation to form a modified tripeptide bound to a resin, which may be further elongated or removed from the resin.

In some embodiments and as understood by a person skilled in the art, similar reaction sequences as presented in Schemes R and S may be conducted using a modified tryptophan having the formula

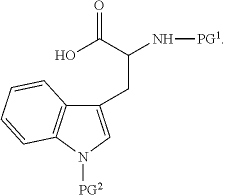

In some embodiments the peptide precursor comprises a terminal primary amino group. In some embodiments the peptide precursor is selected from the group consisting of: an amino acid, a peptide and a solid phase resin. In some embodiments the peptide precursor is a solid phase resin. In some embodiments the peptide precursor is a solid phase resin not coupled to amino acids. In some embodiments the peptide precursor is a solid phase resin coupled to at least one amino acid. In some embodiments the peptide precursor is a peptide. In some embodiments the peptide precursor is an amino acid. In some embodiments the peptide precursor is a solid phase resin having at least one amino acid residue.

In some embodiments the process further comprises step (g) of removing the peptide-based prodrug from the solid phase resin. In some embodiments the process further comprises a step of removing the peptide-based prodrug from the solid phase resin.

In some embodiments the PG¹ is a base-labile protecting group. In some embodiments the PG¹ is fluorenylmethyloxycarbonyl (Fmoc). In some embodiments the PG² is an acid-labile protecting group. The term “acid-labile protecting group” refers to a protecting group that can be removed by treatment with an aqueous or non-aqueous acid. In some embodiments the PG¹ is 4-methyltrityl (Mtt).

In some embodiments the coupling of step (b) comprises contacting the peptide precursor and the protected amino acid in the presence of an amino acid coupling agent. In some embodiments the coupling of step (b) comprises contacting the peptide precursor and the protected amino acid in the presence of a coupling agent selected from a carbodiimide, 1-[Bis(dimethylamino)methylene]-1H-1,2,3-triazolo[4,5-b]pyridinium 3-oxid hexafluorophosphate), 1-Hydroxy-7-azabenzotriazole and combinations thereof. In some embodiments the carbodiimide is dicyclohexyl carbodiimide or diisopropyl carbodiimide. Each possibility represents a separate embodiment.

In some embodiments the peptide-based prodrug comprises at least one carbamate moiety having the formula —NR²CO₂R¹. In some embodiments the peptide-based prodrug comprises at least one amino acid residue comprising a side chain, which comprises NCO₂R¹ and/or NHCO₂R¹ moiety. In some embodiments the peptide-based prodrug comprises at least one amino acid residue comprising a side chain, which comprises —NR²CO₂R¹ moiety. In some embodiments the NR²CO₂R¹ moiety has a formula selected from the group consisting of:

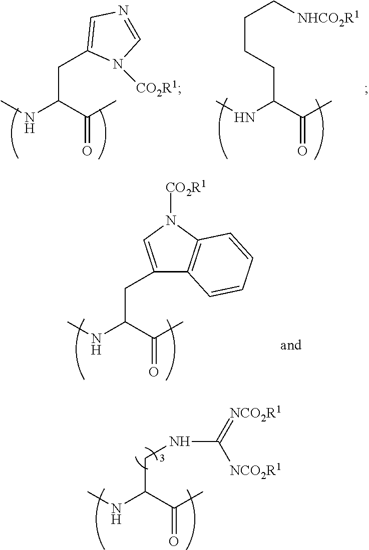

In some embodiments the peptide-based prodrug comprises at least one carbamate moiety having a formula selected from the group consisting of:

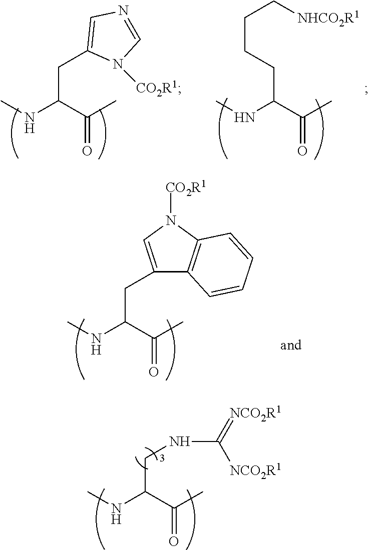

In some embodiments the peptide-based prodrug comprises at least one carbamate moiety having a formula selected from the group consisting of:

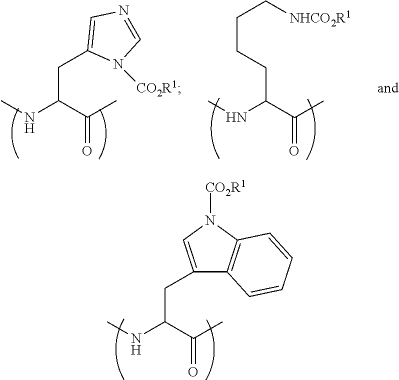

In some embodiments the peptide-based prodrug comprises at least one carbamate moiety having the formula:

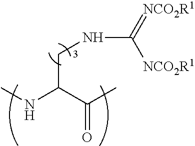

In some embodiments the peptide-based prodrug comprises at least one carbamate moiety having the formula:

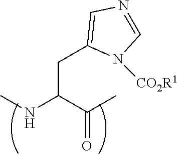

In some embodiments the peptide-based prodrug comprises at least one carbamate moiety having the formula:

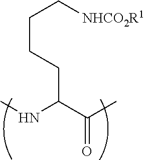

and

In some embodiments the peptide-based prodrug comprises at least one carbamate moiety having the formula:

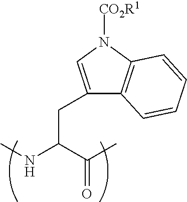

In some embodiments and generally, the transformations presented above (Schemes O, P, R and S) are relating to preparing peptides comprising carbamates as prodrugs of peptides comprising amines. In some embodiments as the amine-containing peptides are basic, they may be protonated under gastrointestinal/physiological pH and thus, the transformations entail inhibiting the prodrug from acquiring positive charge.

In some embodiments the peptide-based prodrug is devoid of positively charged nitrogen atoms. In some embodiments the peptide-based prodrug is devoid of electrically charged nitrogen atoms. In some embodiments the peptide-based prodrug is having a net neutral charge. In some embodiments the peptide-based prodrug is devoid of positively charged atoms. In some embodiments the peptide-based prodrug is devoid of charged atoms. In some embodiments the peptide-based prodrug is devoid of positively charged nitrogen atoms at physiological pH. In some embodiments the peptide-based prodrug is devoid of electrically charged nitrogen atoms at physiological pH. In some embodiments the peptide-based prodrug is having a net neutral charge at physiological pH. In some embodiments the peptide-based prodrug is devoid of positively charged atoms at physiological pH. In some embodiments the peptide-based prodrug is devoid of charged atoms at physiological pH. In some embodiments the peptide-based prodrug is devoid of positively charged nitrogen atoms at gastrointestinal pH. In some embodiments the peptide-based prodrug is devoid of electrically charged nitrogen atoms at gastrointestinal pH. In some embodiments the peptide-based prodrug is having a net neutral charge at gastrointestinal pH. In some embodiments the peptide-based prodrug is devoid of positively charged atoms at gastrointestinal pH. In some embodiments the peptide-based prodrug is devoid of charged atoms at gastrointestinal pH.

In some embodiments step (d) is preformed in the presence of a base selected from trimethylamine and N,N-diisopropylethylamine.

In some embodiments and as mentioned above, it may also be desirable to inhibit negative charge(s) in peptides for enhancing the blood stream permeability of the prodrugs

In some embodiments the peptide precursor of step (a) and/or the at least one additional amino acid comprises at least COOH moiety. In some embodiments the peptide precursor of step (a) and/or the at least one additional amino acid comprises at least one amino acid residue comprising a side chain, which comprises COOH moiety. In some embodiments the peptide precursor of step (a) and/or the at least one additional amino acid comprises at least one amino acid residue selected from the group consisting of aspartic acid, glutamic acid and combinations thereof.

It is to be understood that the esterification may occur before or after the reaction of the starting peptide precursor with the modified amino acid according to some embodiments.

In some embodiments the process further comprises a step of esterifying the COOH moiety. In some embodiments the process further comprises a step of esterifying a COOH containing compound selected from the peptide precursor, the product of step (e) or the product of step (f). In some embodiments the process further comprises a step of esterifying the product of step (e) or (f). In some embodiments the process further comprises a step of esterifying the product of step (f). In some embodiments the process further comprises a step of esterifying the prodrug of step (f). In some embodiments the esterification comprises reacting the COOH containing compound with an alcohol in the presence of an esterification reagent. In some embodiments the esterification reagent is selected from the group consisting of thionyl chloride, oxalyl chloride, phosphorous pentachloride, phosphorous trichloride, phosphoryl chloride, phosgene, diethyl azodicarboxylate (DEAD), diisopropyl azodicarboxylate (DIAD), N,N′-diisopropylcarbodiimide (DIPC), N,N′-dicyclohexylcarbodiimide (DCC) and di-tert-butyl dicarbonate. Each possibility represents a separate embodiment. In some embodiments the esterification reagent is thionyl chloride. In some embodiments the process further comprises a step of reacting the peptide-based prodrug with an alcohol in the presence of thionyl chloride. In some embodiments the process further comprises step (g) of reacting the peptide-based prodrug with an alcohol in the presence of thionyl chloride.

In some embodiments the process further comprises a step of reacting the product of step (e) or (f) with an alkyl chloroformate having the formula ClCO₂R¹.

In some embodiments the peptide-based comprises at least one COOR³ moiety. In some embodiments R³ is other than hydrogen or a metal. In some embodiments R³ is an alkyl group. In some embodiments R³ is an alkyl group selected from methyl, ethyl and isopropyl. In some embodiments R³ is an alkyl group selected from methyl and ethyl. In some embodiments R³ is ethyl.

In some embodiments the COOR³ moiety is a part of an amino acid side chain selected from aspartic acid and glutamic acid.

In some embodiments the peptide-based prodrug comprises no more than a single COOH group. In some embodiments the peptide-based prodrug is devoid of COOH groups.

In some embodiments, there is provided a peptide-based prodrug comprising at least one carbamate moiety, wherein said at least one carbamate moiety is selected from the group consisting of:

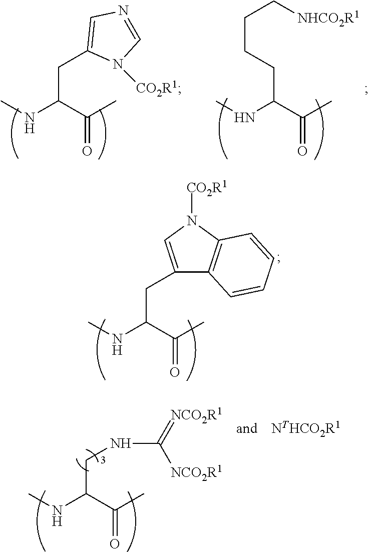

wherein
R¹ is a primary alkyl; and
N^{T} is an N-terminus nitrogen atom of said peptide-based prodrug.

In some embodiments the carbamate moiety has the formula NR²CO₂R¹.

In some embodiments the carbamate moiety has a formula selected from the group consisting of:

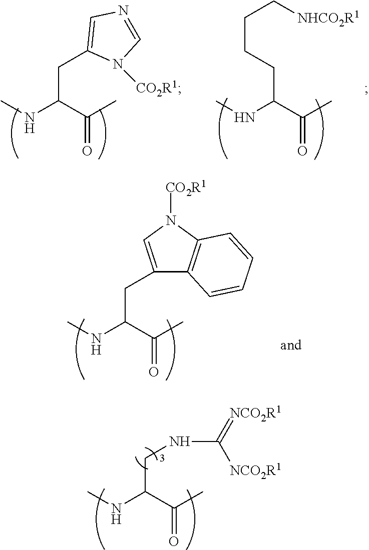

In some embodiments the carbamate moiety has a formula selected from the group consisting of:

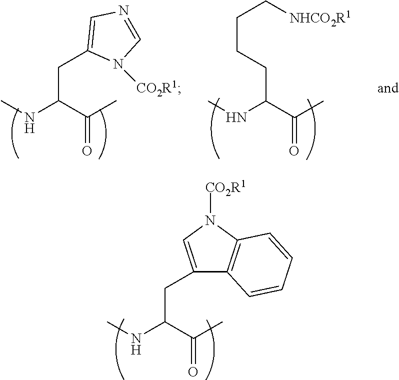

In some embodiments the peptide-based prodrug comprises at least one carbamate moiety, having the formula:

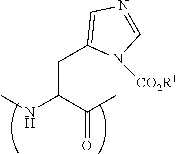

In some embodiments the peptide-based prodrug comprises at least one carbamate moiety, having the formula:

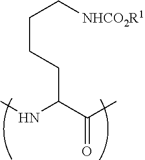

In some embodiments the peptide-based prodrug comprises at least one carbamate moiety, having the formula:

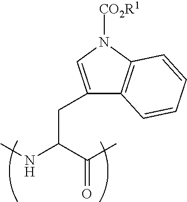

In some embodiments the peptide-based prodrug comprises at least one carbamate moiety, having the formula:

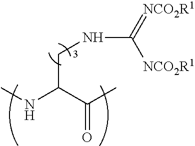

In some embodiments the peptide-based prodrug comprises an N-terminus nitrogen atom having the formula N^{T}HCO₂R¹.

In some embodiments there is provided a peptide-based prodrug comprising an amino terminus comprising a terminal nitrogen atom, a carboxy terminus and at least one —NR²CO₂R¹ moiety, wherein said at least one selected —NR²CO₂R¹ moiety is from the group consisting of:

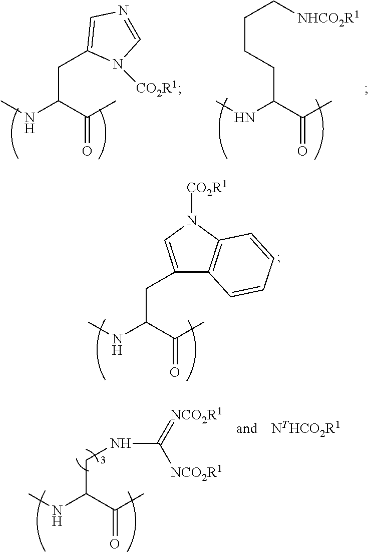

wherein
R¹ is a primary alkyl; and
N^{T} is said terminal nitrogen atom.

In some embodiments and generally, peptides as provided above are characterized by having a lipophilic CO₂R¹ fragment(s). Specifically, the modified amino acid(s), which act as building block(s), provide lipophilic carbamate fragment(s) to the prodrug.

In some embodiments the peptide-based prodrug may be prepared according to any one of the processes described above.

In some embodiments R¹ is a primary alkyl group as defined hereinabove. In some embodiments R² is as defined hereinabove.

In some embodiments, the peptide-based prodrug comprises between 2 and 50 amino acids. In some embodiments, the peptide-based prodrug comprises between 2 and 35 amino acids. In some embodiments, the peptide-based prodrug comprises between 2 and 20 amino acids. In some embodiments, the peptide-based prodrug comprises between 3 and 50 amino acids. In some embodiments, the peptide-based prodrug comprises between 3 and 35 amino acids. In some embodiments, the peptide-based prodrug comprises between 3 and 20 amino acids. In some embodiments, the peptide-based prodrug comprises between 4 and 50 amino acids. In some embodiments, the peptide-based prodrug comprises between 4 and 35 amino acids. In some embodiments, the peptide-based prodrug comprises between 4 and 20 amino acids.

In some embodiments the peptide based prodrug is a cyclic peptide based prodrug.

In some embodiments the cyclic peptide based prodrug has a backbone to backbone intramolecular bond. In some embodiments the cyclic peptide based prodrug has a backbone to backbone intramolecular bond between the N-terminus and the C-terminus of the peptide. In some embodiments the cyclic peptide based prodrug has a backbone to side-chain intramolecular bond. In some embodiments the cyclic peptide based prodrug has a side-chain to side-chain intramolecular bond. In some embodiments the cyclic peptide based prodrug has a side-chain to side-chain intramolecular disulfide bond between two cysteine side chain residues. In some embodiments the cyclic peptide-based prodrug does not include an amino terminus.

In some embodiments the cyclic peptide is somatostatin or a somatostatin analog.

In some embodiments the peptide-based prodrug comprises at least two —NR²CO₂R¹ moieties. In some embodiments the peptide-based prodrug comprises at least three —NR²CO₂R¹ moieties. In some embodiments the peptide-based prodrug comprises at least four —NR²CO₂R¹ moieties.

In some embodiments the peptide-based prodrug comprises no more than a single primary amine group. In some embodiments the peptide-based prodrug is devoid of primary amine groups. It is to be understood that the “primary amine group(s)” refers to amines, and does not include amides, thus, for example, peptides which include primary —CONH₂ group(s) may still be devoid from primary amino groups.

In some embodiments the peptide-based prodrug comprises histidine, arginine, tryptophan and/or lysine residue(s), each of said residues comprises an —NR²CO₂R¹ moiety.

In some embodiments the —NR²CO₂R¹ moiety has the formula:

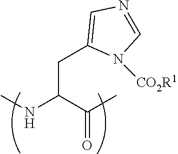

In some embodiments the —NR²CO₂R¹ moiety has the formula:

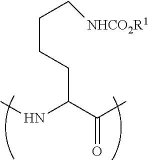

In some embodiments the —NR²CO₂R¹ moiety has the formula:

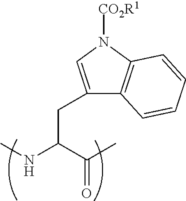

In some embodiments the —NR²CO₂R¹ moiety has the formula:

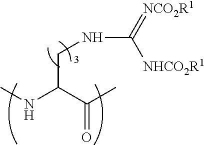

In some embodiments the —NR²CO₂R¹ moiety has the formula N^{T}HCO₂R¹.

In some embodiments the peptide-based prodrug comprises at least one amino acid residue comprising a side chain, which comprises the —NR²CO₂R¹ moiety.

As used herein, the term “amino terminus” (abbreviated N-terminus) refers to a free or modified (such as NHCO₂-alkyl) α-amino group (moiety) at the amino terminal of a peptide or a peptide-based prodrug. The term “terminal nitrogen atom” refers to the nitrogen atom of said amino terminus.

Similarly, the term “carboxy terminus” refers to the free or esterified carboxyl group on the carboxy terminus of a peptide or a peptide-based prodrug.

In some embodiments the peptide-based prodrug is devoid of positively charged nitrogen atoms. In some embodiments the peptide-based prodrug is devoid of electrically charged nitrogen atoms. In some embodiments the peptide-based prodrug is having a net neutral charge. In some embodiments the peptide-based prodrug is devoid of positively charged atoms. In some embodiments the peptide-based prodrug is devoid of charged atoms. In some embodiments the peptide-based prodrug is devoid of positively charged nitrogen atoms at physiological pH. In some embodiments the peptide-based prodrug is devoid of electrically charged nitrogen atoms at physiological pH. In some embodiments the peptide-based prodrug is having a net neutral charge at physiological pH. In some embodiments the peptide-based prodrug is devoid of positively charged atoms at physiological pH. In some embodiments the peptide-based prodrug is devoid of charged atoms at physiological pH. In some embodiments the peptide-based prodrug is devoid of positively charged nitrogen atoms at gastrointestinal pH. In some embodiments the peptide-based prodrug is devoid of electrically charged nitrogen atoms at gastrointestinal pH. In some embodiments the peptide-based prodrug is having a net neutral charge at gastrointestinal pH. In some embodiments the peptide-based prodrug is devoid of positively charged atoms at gastrointestinal pH. In some embodiments the peptide-based prodrug is devoid of charged atoms at gastrointestinal pH.

In some embodiments the peptide-based comprises at least one CH₂COOR³ moiety. In some embodiments R³ is other than hydrogen or a metal. In some embodiments R³ is an alkyl group. In some embodiments R³ is an alkyl group selected from methyl, ethyl and isopropyl. In some embodiments R³ is an alkyl group selected from methyl and ethyl. In some embodiments R³ is ethyl.

In some embodiments the CH₂COOR³ moiety is a part of an amino acid side chain selected from aspartic acid and glutamic acid.

In some embodiments the peptide-based prodrug comprises no more than a single COOH group. In some embodiments the peptide-based prodrug is devoid of COOH groups.

Pharmaceutical compositions comprising at least one peptide based prodrug as disclosed herein are provided.

Pharmaceutical compositions for use in accordance with the present invention may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

According to some embodiments, the pharmaceutical compositions are formulated for oral administration.

According to other embodiments, the pharmaceutical compositions are formulated for parenteral administration.

According to some embodiments the formulation further comprises an excipient, carrier or diluent suitable for oral or parenteral administration. Suitable pharmaceutically acceptable excipients for use in this invention include those known to a person ordinarily skilled in the art such as diluents, fillers, binders, disintegrants and lubricants. Diluents may include but not limited to lactose, microcrystalline cellulose, dibasic calcium phosphate, mannitol, cellulose and the like. Binders may include but not limited to starches, alginates, gums, celluloses, vinyl polymers, sugars and the like. Lubricants may include but not limited to stearates such as magnesium stearate, talc, colloidal silicon dioxide and the like.

According to some embodiments, a pharmaceutical composition according to the present invention comprises at least one absorption enhancer, such as but not limited to, nanoparticles, piperine, curcumin and resveratrol.

According to some embodiments the pharmaceutical composition comprises a delivery system selected from the group consisting of: a Pro-NanoLipospheres (PNL) composition, an Advanced PNL and a self-nano emulsifying drug delivery system (SNEDDS).

The pharmaceutical compositions and the uses of the present invention may comprise, according to some embodiments, at least one additional active agent.

The following non-limiting examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### Examples

### Material and Methods

### Chromatography

Semi-preparative reversed phase HPLC was performed using Waters instruments: Waters 2545 (Binary Gradient Module), Waters SFO (System Fluidics Organizer), Waters 2996 (Photodiode Array Detector), Waters 2767 (Sample Manager). Dr. Maisch C18-column: Reprosil 100 C18, 5 μm, 150×30 mm was used. The Semi-preparative RP-HPLC were operated with a flow rate of 40 mL/min with a linear gradient (20 min) of H₂O (0.1% v/v trifluoroacetic acid (TFA)) and acetonitrile (0.1% v/v TFA). Analytical HESI HPLC-MS (heated electrospray ionization mass spectrometry) was performed on a LCQ Fleet (Thermo Scientific) with a connected UltiMate 3000 UHPLC focused (Dionex) on C18-columns: S1: Hypersil Gold aQ 175 Å, 3 μm, 150×2.1 mm (for 8 or 20 minutes measurements); S2: Accucore C18, 80 Å, 2.6 μm, 50×2.1 mm (for 5 minute measurements) (Thermo Scientific). Linear gradients (5% 95% acetonitrile content) with H₂O (0.1% v/v formic acid) and acetonitrile (0.1% v/v formic acid) as eluents were used.

### NMR

All NMR resonances were assigned in DMSO-d6 at 298 K (except the temperature gradient resonances) and at proton resonance frequency of 400 MHz or 500 MHz. Chemical shifts are referenced to the DMSO 1H resonance at 2.50 ppm and the DMSO 13CMe resonance 39.51 ppm.

### Synthesis of Cyclic Peptides

Loading of CTC-resin. Peptide synthesis was carried out using CTC-resin (0.9 mmol/g) following standard Fmoc-strategy. Fmoc-Xaa-OH (1.2 eq.) were attached to the CTC-resin with N,N-diisopropylethylamine (DIEA; 2.5 eq.) in anhydrous DCM (0.8 mL/g resin) at room temperature (rt) for 1 h. The remaining trityl-chloride groups were capped by addition of a solution of MeOH (1 mL/g (resin)), DIEA (5:1; v:v) for 15 min. The resin was filtered and washed 5 times with DCM and 3 times with MeOH. The loading capacity was determined by weight after drying the resin under vacuum and ranged from 0.4-0.9 mmol/g.

On-resin Fmoc-Deprotection. The Fmoc peptidyl-resin was treated with 20% piperidine in NMP (v/v) for 10 minutes and a second time for 5 minutes. The resin was washed 5 times with NMP.

Standard Amino Acid Coupling. A solution of Fmoc-Xaa-OH (2 eq.), O-(7-azabenzotriazole-lyl)-N,N,N′,N′-tetramethyluronium-hexafluorophosphate (HATU) (2 eq.), 1-hydroxy-7-azabenzotriazole (HOAt; 2 eq.), and DIEA (3 eq.) in NMP (1 mL/g resin) was added to the free amino peptidyl-resin and shaken for 60 min at room temperature and washed 5 times with NMP.

On-Resin N-Methylation. The linear Fmoc-deprotected peptide was washed with DCM (3×) incubated with a solution of 2-nitrobenzenesulfonylchloride (o-Ns-Cl, 4 eq.) and 2,4,6-Collidine (10 eq.) in DCM for 20 min at room temperature. The resin was washed with DCM (3×) and THF abs. (5×). A solution containing PPh₃ (Seq.) and MeOH abs. (10 eq.) in THF abs. was added to the resin. DIAD (5 eq.) in a small amount THF abs. is added stepwise to the resin and the solution was incubated for 15 min and washed with THF (5×) and NMP (5×). For o-Ns deprotection, the o-Ns-peptidyl-resin was stirred in a solution of mercaptoethanol (10 eq.) and DBU (5 eq.) in NMP (1 mL/g resin) for 5 minutes. The deprotection procedure was repeated once more and the resin was washed 5 times with NMP.

Cleavage of Linear Peptides from Resin. For complete cleavage from the resin the peptides were treated three times with a solution of DCM and hexafluoroisopropanol (HFIP; 4:1; v:v) at room temperature for half an hour and the solvent evaporated under reduced pressure.

Cyclization with Diphenylphosphoryl Azide (DPPA). To a solution of peptide in DMF (1 mM peptide concentration) and NaHCO3 (5 eq.) DPPA (3 eq.) was added at room temperature (rt) and stirred over night or until no linear peptide could be observed by HPLC-MS. The solvent was evaporated to a small volume under reduced pressure and the peptides precipitated in saturated NaCl solution and washed two times in HPLC grade water.

Removal of Acid Labile Side Chain Protecting Group. Cyclized peptides were stirred in a solution of TFA, water and TIPS (95:2.5:2.5) at room temperature for one hour or until no more protected peptide could be observed by HPLC-MS and precipitated in diethylether. The precipitated peptide was collected after centrifugation and decantation. This precipitated peptide was washed with diethylether and collected two more times.

Dde-Deprotection in Solution. The orthogonal deprotection of the Dde-protecting group (1-(4,4-dimethyl-2,6-dioxocyclohex-1-ylidene)ethyl) was performed using 2 vol % solution of hydrazine hydrate in dimethylformamide (DMF) for 30 min at room temperature. The progress of the reaction was monitored by HPLC-MS. After completion of the reaction, the peptide was precipitated with sat. aq. NaCl-solution and washed two times with water.

Guanidinylation in Solution. The Dde-deprotected peptide were stirred in a solution of 1H-Pyrazole-carboxamidine-hydrchloride (2.0 eq.) and DIEA (3.0 eq.) at room temperature for 12 hours. The progress of the reaction was controlled via HPLC-MS. After completion, the solvent was removed under reduced pressure.

Reductive Deprotection. The orthogonal deprotection of the benzyl-group via hydrogenolysis was performed using a palladium catalyst on activated carbon (10% Pd/C with 50% H₂O as stabilizer, 15 mg/mmol) and hydrogen atmosphere (1 atm. H₂) at room temperature. The completion of the deprotection was monitored by HPLC-MS, the catalyst was removed over diatomaceous earth and the solvent was removed under pressure.

### Synthesis of Hoc-Protected Arginine

Trimethylsilyl (TMS) Protection of Carboxylic acid. To dry Fmoc-protected Arginine DCM and DIEA (4. eq.) was added under argon atmosphere. With continuous stirring TMSCl (4 eq.) was added in 2-4 portions to the solution and was stirred at 40° C. for 1.5 h with a refluxing condenser. This resulted in a TMS-protected Fmoc-Arginine.

Hexyloxycarbonyl (Hoc) Protection. The solution of TMS-protected Fmoc-Arginine was cooled to 0° C. and it was added DIEA (3 eq.) followed by the stepwise addition of hexyl chloroformiate (3 eq.). The solution was stirred at 0° C. for 30 mins, then raised to room temperature and was stirred overnight. The completion was confirmed by HPLC-MS.

Removal of TMS. The reaction contents were acidified by addition of 1N HCl until the pH of the organic layer was 2 and hence the deprotection of the TMS group. The compound, Fmoc-Arg(Hoc)2-OH, was extracted with DCM (3-5×), the extracts were then combined, dried with MgSO4 and DCM was removed afterwards under reduced pressure. The final product was obtained after crystallization from a solution of methanol and water (4:1; v/v) and was confirmed by HPLC-MS.

### Integrin Binding Assay

The activity and selectivity of integrin ligands were determined by a solid-phase binding assay, applying a previously described protocol [11, 12], using coated extracellular matrix proteins and soluble integrins. The following compounds were used as internal standards: Cilengitide, (SEQ ID NO: 20), c(f(NMe)VRGD) (αvβ3—0.54 nM, αvβ5—8 nM, α5β1—15.4 nM), linear peptide RTDLDSLRT4 (SEQ ID NO: 24) (αvβ6—33 nM; αvβ8—100 nM) and tirofiban5 (αIIbβ3—1.2 nM). Flat-bottom 96-well ELISA plates (BRAND, Wertheim, Germany) were coated overnight at 4° C. with the ECM-protein (1) (100 μL per well) in carbonate buffer (15 mM Na₂CO₃, 35 mM NaHCO₃, pH 9.6). Each well was then washed with PBS-T-buffer (phosphate-buffered saline/Tween20, 137 mM NaCl, 2.7 mM KCl, 10 mM Na₂HPO₄, 2 mM KH₂PO₄, 0.01% Tween20, pH 7.4; 3×200 μL) and blocked for 1 h at room temperature (rt) with TS-B-buffer (Tris-saline/BSA buffer (bovine serum albumin); 150 μL/well; 20 mM Tris-HCl, 150 mM NaCl, 1 mM CaCl₂), 1 mM MgCl₂, 1 mM MnCl₂, pH 7.5, 1% BSA). In the meantime, a dilution series of the compound and internal standard is prepared in an extra plate, starting from 20 μM to 6.4 nM in 1:5 dilution steps. After washing the assay plate three times with PBS-T (200 μL), 50 μl of the dilution series were transferred to each well from B-G. Well A was filled with 100 μl TSB-solution (blank) and well H was filled with 50 μl TS-B-buffer. 50 μl of a solution of human integrin (2) in TS-B-buffer was transferred to wells H-B and incubated for 1 h at room temperature (rt). The plate was washed three times with PBS-T buffer, and then primary antibody (3) (100 μL per well) was added to the plate. After incubation for 1 h at rt, the plate was washed three times with PBS-T. Then, secondary peroxidase-labeled antibody (4) (100 μL/well) was added to the plate and incubated for 1 h at rt. After washing the plate three times with PBS-T, the plate was developed by quick addition of SeramunBlau (50 μL per well, Seramun Diagnostic GmbH, Heidesee, Germany) and incubated for 5 min at rt in the dark. The reaction was stopped with 3 M H₂SO₄ (50 μL/well), and the absorbance was measured at 405 nm with a plate reader (GENios, TECAN).

The IC₅₀-value of each compound was tested in duplicate and the resulting inhibition curves were analyzed using OriginPro 9.0G software. The inflection point describes the IC₅₀-value. All determined IC₅₀-values were referenced to the activity of the internal standard.

αvβ3

(1) 1.0 μg/mL human vitronectin; Millipore.

(2) 2.0 μg/mL, human αvβ3-integrin, R&D.

(3) 2.0 μg/mL, mouse anti-human CD51/61, BD Biosciences.

(4) 2.0 μg/mL, anti-mouse IgG-POD, Sigma-Aldrich.

α5β1

(1) 0.5 μg/mL; human fibronectin, Sigma-Aldrich.

(2) 2.0 μg/mL, human α5β1-integrin, R&D.

(3) 1.0 μg/mL, mouse anti-human CD49e, BD Biosciences.

(4) 2.0 μg/mL, anti-mouse IgG-POD, Sigma-Aldrich.

αvβ5

(1) 5.0 μg/mL; human vitronectin, Millipore.

(2) 3.0 μg/mL, human αvβ5-integrin, Millipore.

(3) 1:500 dilution, anti-av mouse anti-human MAB1978, Millipore.

(4) 1.0 μg/mL, anti-mouse IgG-POD, Sigma-Aldrich.

αvβ6

(1) 0.4 μg/mL; LAP (TGF-3), R&D.

(2) 0.5 μg/mL, human αvβ6-Integrin, R&D.

(3) 1:500 dilution, anti-α v mouse anti-human MAB1978, Millipore.

(4) 2.0 μg/mL, anti-mouse IgG-POD, Sigma-Aldrich.

αvβ8

(1) 0.4 μg/mL; LAP (TGF-b), R&D.

(2) 0.5 μg/mL, human αvβ8-Integrin, R&D.

(3) 1:500 dilution, anti-av mouse antihuman MAB1978, Millipore.

(4) 2.0 μg/mL, anti-mouse IgG-POD, Sigma-Aldrich.

αIIbβ3

(1) 10.0 μg/mL; human fibrinogen, Sigma-Aldrich.

(2) 5.0 μg/mL, human platelet integrin αIIβ3, VWR.

(3) 2.0 μg/mL, mouse anti-human CD41b, BD Biosciences.

(4) 1.0 μg/mL, anti-mouse IgG-POD, Sigma-Aldrich.

### Permeability Study

Culture of colorectal adenocarcinoma 2 (Caco-2) cells. Caco-2 cells (ATTC) were grown in 75 cm² flasks with approximately 0.5×10⁶ cells/flask (Thermo-Fischer) at 37° C. in a 5% CO₂ atmosphere and at relative humidity of 95%. The culture growth medium consisted of DMEM supplemented with 10% heat-inactivated FBS, 1% MEM-NEAA, 2 mM 1-glutamine, 1 mM sodium pyruvate, 50,000 units Penicillin G Sodium and 50 mg Streptomycin Sulfate (Biological Industries). The medium was replaced every other day.

Caco-2 cells growth and treatment. Cells (passage 55-60) were seeded at density of 25×10⁵ cells/cm² on untreated culture inserts of polycarbonate membrane with 0.4 μm pores and surface area of 1.1 cm². Culture inserts containing Caco-2 monolayer were placed in 12 mm transwell plates (Corning). Culture medium was replaced every other day. Transepithelial Electrical Resistance (TEER) values were measured by Millicell ERS-2 System (Millipore) a week after seeding up to experiment day (21-23 days) to ensure proliferation and differentiation of the cells. When the cells were fully differentiated and TEER values became stable (200-500 Ω·cm²). The TEER values were compared to control inserts containing only the medium.

In vitro permeability studies using Caco-2 cells. The experiment was initiated by replacing the medium from both sides by apical (600 μl) and basolateral (1500 μl) buffers, both warmed to 37° C. The Cells were incubated with the buffers solutions for 30 min at 37° C. on a shaker (100 cycles/min). The apical buffer was replaced by apical buffer containing 10 μg/ml 29 (SEQ ID NO: 5) or 10 μg/ml 29P (SEQ ID NO: 9). 50 μl samples were taken from the apical side immediately at the beginning of the experiment, resulting in 550 μl apical volume during the experiment. Samples of 200 μl at fixed time points (20, 40, 60, 80, 100, 120 and 150 min) from the basolateral side and replaced with the same volume of fresh basolateral buffer to maintain a constant volume. The experiment included two control compounds, atenolol and metoprolol, as paracellular and transcellular permeability markers.

Caco-2 permeability study data analysis. Permeability Coefficient (Papp) for each compound was calculated from the linear plot of drug accumulated versus time, using the following equation:

$$Papp = \frac{dq / dt}{{C}_{0} \times A}$$

Where dq/dt is steady state appearance rate of the compound on the receiver side, C₀ is the initial concentration of the drug on the donor side, and A is the exposed tissue surface area (1.1 cm²).

Enzymatic inhibition studies. For the determination of enzymatic inhibition by the self-nano emulsifying drug delivery system (SNEDDS) [13] or ketoconazole, pooled rat CYP3A4 microsomes (BD Biosciences, Woburn, Mass., USA) were used. The reaction was initiated by adding ice cold microsomes (0.5 mg/mL final concentration) to a preheated phosphate buffer (0.1M, pH 7.4) containing NADPH (0.66 mg/mL) and dispersed 12P (SEQ ID NO: 10)-SNEDDS (2.8 μL, equivalent to 12P 1 μM), with ketoconazole (3 μM) or 12P (SEQ ID NO: 10) alone (1 μM). At predetermined times (0, 15, and 30 min), 50 μL samples were withdrawn, and the reaction was terminated by adding 100 μL of ice cold ACN and further processed as described in the Analytical Methods section below.

In Vivo Studies. Male Wistar rats (Harlan, Israel), 275-300 g in weight, were used for all surgical procedures. Animals were anesthetized for the period of surgery by intraperitoneal injection of 1 mL/kg of ketamine/xylazine solution (9:1), placed on a heated surface, and maintained at 37° C. (Harvard Apparatus Inc., Holliston, Mass.). An indwelling cannula was placed in the right jugular vein of each animal for systemic blood sampling, by a method described before. The cannula was tunneled beneath the skin and exteriorized at the dorsal part of the neck. After completion of the surgical procedure, the animals were transferred to cages to recover overnight (12-18 h). During this recovery period, food but not water was deprived. Throughout the experiment, free access to food was available 4 h post oral administration. Animals were randomly assigned to the different experimental groups. For bioavailability studies, dispersed 12P SNEDDS was freshly prepared 30 min before each experiment by vortex-mixing of the preconcentrate in water (1:10, v/v) preheated to 37° C. for 30 s. Dispersed 12P SNEDDS (5 mg/kg) was administered to the animals by oral gavage (n=3). Systemic blood samples (0.35 mL) were taken at 5 min predose, 20, 40, 60, 90, 180, 240, and 360 min postdose. To prevent dehydration, equal volumes of physiological solution were administered to the rats following each withdrawal of blood sample. Plasma was separated by centrifugation (5322 g, 10 min) and stored at −20° C. pending analysis. In the 12P pharmacokinetic study, the parent peptide, 12, was analytically determined.

Pharmacokinetic Analysis. The area under the plasma concentration-time curve (AUC) was calculated by using the trapezoidal rule with extrapolation to infinity by dividing the last measured concentration by the elimination rate constant (kel). The elimination rate constant values were determined by a linear regression analysis using the last points on the logarithmic plot of the plasma concentration versus the time curve. Pharmacokinetic parameters, such Tmax, Cmax, clearance (CL), volume of distribution (V), and bioavailability, were calculated using noncompartmental analysis.

Analytical Methods. Plasma or BBMV samples were spiked with metoprolol (1.5 μg/mL) as an internal standard. ACN was added to each sample (2:1) and vortex-mixed for 1 min. The samples were then centrifuged (14 635 g, 10 min), and the supernatant was transferred to fresh glass tubes and evaporated to dryness (Vacuum Evaporation System, Labconco, Kansas City, Mo., USA). Then, the glass tubes were reconstituted in 80 μL of mobile phase and centrifuged a second time (14 635 g, 10 min). The amount of the compounds was determined using an HPLC-MS Waters 2695 Separation Module, equipped with a Micromass ZQ detector. The resulting solution was injected (10 μL) into the HPLC system. The system was conditioned as follows: for parent drug peptides (including 12), a Kinetex 2.6 μm HILIC 100 Å, 100 mm×2.1 mm column (Phenomenex, Torrance, Calif., USA), an isocratic mobile phase, and an acetonitrile:water:ammonium acetate buffer 50 mM (70:10:20, v/v/v) was used; and for the prodrug peptides (including 12P), a Luna (Phenomenex) 3 μm C8 100 Å, 100 mm×2.0 mm column and an isocratic mobile phase of ACN:water supplemented with 0.1% formic acid (70:30, v/v) and a flow rate of 0.2 mL/min at 25° C. was used. The limit of quantification for all of the peptides and prodrugs was 25 ng/mL.

Statistical Analysis. All values are expressed as mean±standard error of the mean (SEM) if not stated otherwise. To determine statistically significant differences among the experimental groups, a t-test or one-way ANOVA, followed by Tukey's test, was used. A p-value of less than 0.05 was termed significant.

### Example 1: Screening of Peptide Libraries with Spatial Diversity for Highly Active and Selective RGD Containing N-Methylated Cyclic Hexapeptides

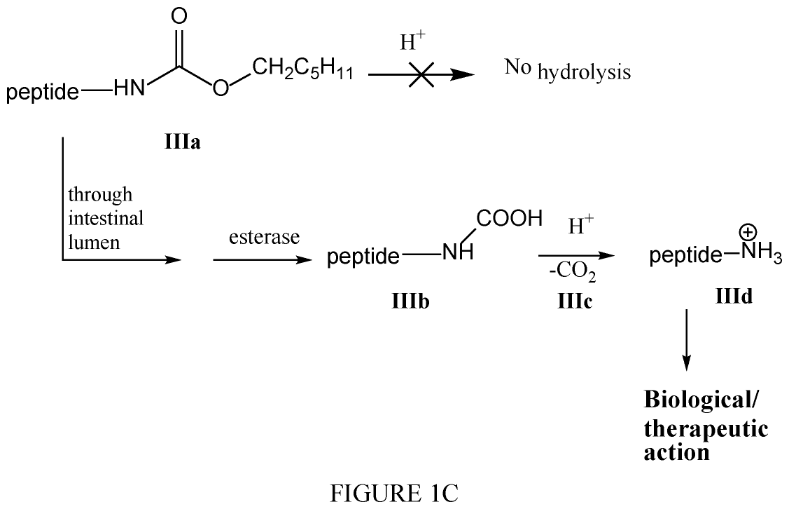

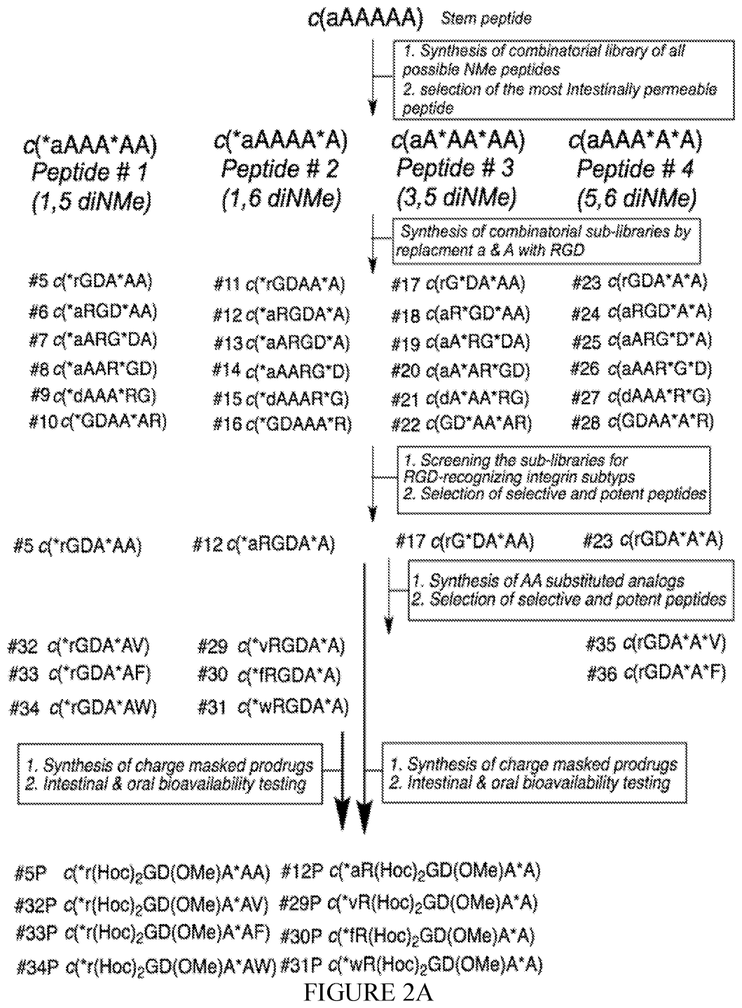

The method as well as number and sequence of each peptide are depicted in the flowchart shown in FIG. 2A.

Step 1. Synthesis of Combinatorial Library of all Possible N-Methylated Analogs of the Stem Peptide cyclo(D-Ala-Alas) (c(aAAAAA), SEQ ID NO: 19) and Selection of the Cyclic Peptide with Highest Intestinal Permeability.

The structure-permeability relationship (SPR) of a combinatorial library of 54 out of 63 possible all Ala cyclic hexapeptides c(aAAAAA) with different N-methylation pattern was evaluated. The peptides with highest permeability were chosen as templates for “refunctionalization”. It was found that these peptides strongly vary in permeability, some of them exhibiting an extremely high Caco-2 permeability or even higher comparable to the Caco-2 standard testosterone [2] (peptides 1-4, FIG. 2B). It turned out that the permeability of cyclic hexapeptides is strongly dependent on their molecular structure [5, 6] and clearly provide evidence that participation of a transporter is responsible for the high permeability of some of these peptides. We have shown that the Caco-2 permeability does not correlate with one single parameter such as i) the number of N-methylated amino acids, ii) the number of externally oriented NH groups [2] and iii) the lipophilicity. The peptides with the highest permeability turned out to be a subgroup of peptides with twofold N-methylation in distinct positions: the 1,5-; the 1,6-; the 3,5- and the 5,6-dimethylated peptide (Peptides 1-4, FIGS. 2A and 2B) [1, 2]. Another highly permeable peptide c(*aAA*A*A*A) with the fourfold N-methylation pattern (NMe 1,4,5,6) was not used as a scaffold since it is chemically less stable and synthetically more difficult to prepare.

Step 2. Synthesis of Sub-Libraries of Each of the Selected Cyclic Peptide that Includes the RGD Sequence in all Possible Positions.

The most permeable scaffolds (peptides 1-4, FIGS. 2A and 2B) were used for the construction of second generation combinatorial sub-libraries in which Ala side chains were replaced by side chains of amino acids derived from the active regions of peptides or proteins. The three consecutive Ca methyl groups were systematically replaced (or omitted for G) by the RGD side chains. This manipulation allows the presentation of the RGD side chains in very different spatial orientations that are impossible to predict from the knowledge of several X-ray structures of integrin head groups with bound peptidic ligands [7-10].

Step 3. Selection of the Best Ligands for RGD-Recognizing Integrin Subtypes.

Twenty-four (#5-#28 in FIG. 2A) RGD peptides were screened for their binding to various RGD binding integrins. The results of selected peptides are shown in Table 1. It turned out that only very few compounds had low nanomolar affinity for binding to the integrin subtype αvβ3 and only one to two orders of magnitude lower affinity for α5β1. This is remarkable as linear RGD containing peptides usually bind with some affinity also to some of the other RGD binding integrins (αvβ5, αvβ6, αvβ8 and αIIbβ3) [11]. One exception is the family of the (3,5)-NMe peptides (Peptide #17-22) that show low affinity for all integrin subtypes. The parent (3,5)-NMe all Ala peptide (peptide 3) exhibited two conformations in the NMR spectrum (in DMSO solution), in contrast to the 1,5- and 1,6-dimethylated parent peptides (peptides 1 and 2) that are conformational homogeneous on the NMR time scale. Obviously the two conformations of peptide 3 are cis/trans isomers around one or more peptide bonds.

TABLE 1
IC₅₀-values of peptide ligands for RGD-recognizing integrin subtypes αvβ3, αvβ5,
αvβ6, α5β1.

| | peptide | | | | | |
|---|---|---|---|---|---|---|
| | name or | | αvβ3, | αvβ5, | αvβ6, | α5β1, |
| scaffold | # | sequence | IC₅₀ [nM] | IC₅₀ [nM] | IC₅₀ [nM] | IC₅₀ [nM] |
| | cilengitide | c(f*VRGD) | 0.61 ± 0.06 | 8.4 ± 2.1 | 2050 ± 640 | 15 ± 3 |
| NMe(1, 5) | 5 | c(*rGDA*AA) | 13 ± 2 | 170 ± 30 | 25 ± 2.5 | 37 ± 4 |
| NMe(1, 6) | 12 | c(*aRGDA*A) | 4.8 ± 1.8 | 1500 ± n.d. | 770 ± n.d. | 200 ± 60 |
| NMe(3, 5) | 17 | c(rG*DA*AA) | 2350 ± 210 | >5000 | >10000 | >10000 |
| NMe(5, 6) | 23 | c(rGDA*A*A) | 73 ± 15 | n.d. | 130 ± 11 | 76 ± 6 |
| NMe(1, 6) | 29 | c(*fRGDA*A) | 0.6 ± 0.2 | 430 ± n.d. | 290 ± n.d. | 35 ± 5 |
| NMe(1, 6) | 30 | c(*vRGDA*A) | 0.6 ± 0.2 | 145 ± n.d. | 120 ± n.d. | 21 ± 2 |
| NMe(1, 5) | 33 | c(*rGDA*AF) | 4.4 ± 1.1 | n.d. | 25 ± 3 | 43 ± 4 |
| NMe(1, 5) | 32 | c(*rGDA*AV) | 5.6 ± 1.8 | n.d. | 3.8 ± 0.6 | 20 ± 2 |

*cilengitide is SEQ ID NO: 20, peptide #5 is SEQ ID NO: 1, peptide #12 is SEQ ID NO: 2, peptide #17 is SEQ ID NO: 3, peptide #23 is SEQ ID NO: 4, peptide #29 is SEQ ID NO: 5, peptide #30 is SEQ ID NO: 6, peptide #32 is SEQ ID NO: 7, and peptide #33 is SEQ ID NO: 8.

Step 4. Fine Tuning of the Best Ligands by Additional Ala to Xaa Substitution for Optimization of Affinity and Selectivity;

The next step was the optimization of the most active peptides by replacement of Ala residues flanking to the of RGD motif. It is known from many structure activity relationship (SAR) studies that aromatic residues flanking the RGD sequence enhance affinity and selectivity towards members of the RGD recognizing integrin subfamily, see e.g. [12]. For example, substitution of the D-Ala residue in peptide 12 by D-Phe and D-Val residues resulted in ligands (peptides 29 and 30) with subnanomolar affinity for αvβ3 with an almost two orders of magnitude lower affinity for α5β1 (Table 1). The affinity and selectivity of the new compounds are comparable or even better than Cilengitide.

Step 5. Protection of the Charged Functional Groups by the Prodrug Concept to Regain Intestinal and Oral Permeability of the Active Peptide.

Peptides #5, 12, 17, 23, 29 and 30 were tested for intestinal permeability in the Caco-2 model. It turned out that all peptides had significant lower permeability than their parent all Alanine-peptides (peptides #1-4). This loss of permeability may attributed to the interdiction of the charged guanidinium and carboxylate groups of the RGD tripeptide sequence. Indeed, the introduction of a single carboxyl group (aspartic acid instead of Ala) or a single guanidinium group (Arg instead of Ala) in any position of peptide #1 (altogether 2×6 peptides) reduced permeability completely. To enable intestinal and oral bioavailability of the RGD containing peptide selected in steps iv and v it is essential therefore to mask the charges of both Arg and Asp. For this purpose, the prodrug approach was applied, in which the charged residues are masked by lipophilic pro-moieties that are cleaved by esterases. The charge on the Asp residue masked with methyl ester pro-moiety, and the charge of the guanidium group of Arg masked with the dihexyloxycarbonyl pro-moiety. Specifically, guanidine group of the Arg residue of the prodrug described in the following examples was masked with two hexyloxycarbonyl (Hoc) moieties and the carboxylic side chain of Asp was transformed into the neutrally charged methyl ester (OMe). Both lipophilic alkyl pro-moieties contain an ester bond. Thus, the prodrugs are readily bioconverted to their original active peptide by ubiquitous esterases, that are presented throughout the body.

### Example 2: Intestinal Permeability, Metabolic Stability and Oral Bioavailability Studies

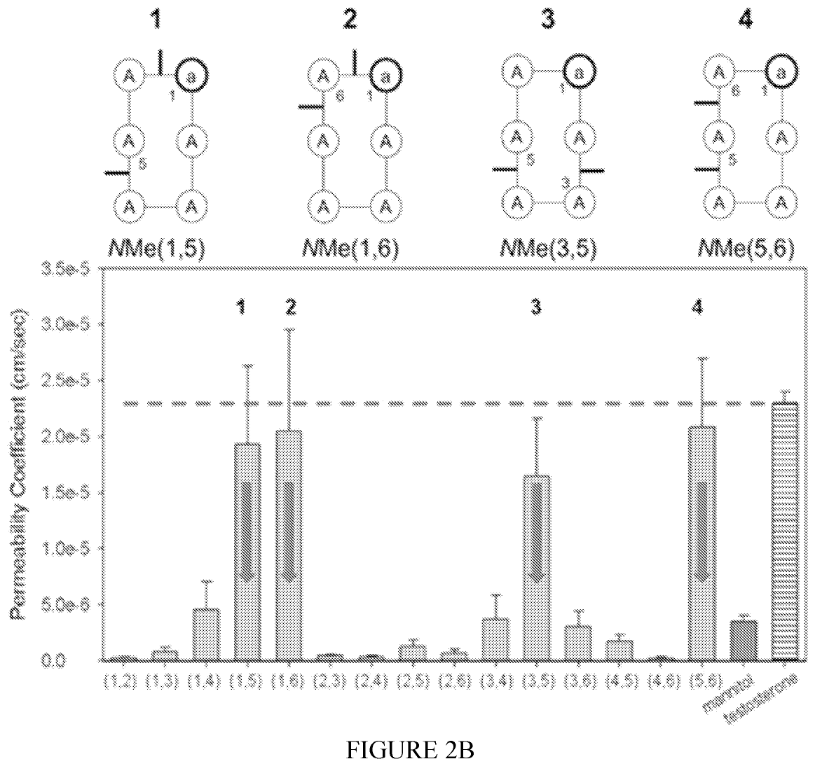

For the proof of concept of the prodrug method peptide 12 (SEQ ID NO: 2) and its prodrug peptide 12P (SEQ ID NO: 10) were used (FIGS. 3A and 3B).

In-vitro permeability studies utilized with the Caco-2 model are an essential component of designing the DLP of peptides, as they allow good prediction for in-vivo oral absorption of compounds [13]. The Caco-2 model is a widely used tool in the academia and pharmaceutical industry to evaluate and predict compounds' permeability mechanism. The Caco-2 system consists of human colon cancer cells that multiply and grow to create a monolayer that emulate the human small intestinal mucosa [14].

Transport studies were performed through the Caco-2 monolayer mounted in an Ussing-type chamber set-up with continuous trans-epithelial electrical resistance (TEER) measurements to assure TEER between 800 and 1200 Ω*cm². HBSS supplemented with 10 mM IVIES and adjusted to pH 6.5 were used as transport medium in the donor compartment and pH 7.4 in the acceptor compartment. The donor solution contained the test compound. The effective permeability coefficients (Papp) were calculated from concentration-time profiles of each of the tested compounds in the acceptor chamber [15]. In every assay, the compounds were compared to the standards atenolol and metoprolol which represent para-cellular and trans-cellular permeability mechanisms respectably [16].

Permeability mechanism of compounds is studied by evaluating the Papp of a compound from the apical to the basolateral (A-to-B) membrane and its Papp from the basolateral to the apical membrane (B-to-A). The A-to-B assay simulates passive and transporter-mediated permeability. The B-to-A assay is essential complementary experiment indicative of the activity of P-gp. The ratio of the A-to-B and B-to A Papps (efflux ratio) is calculated to determine the permeability mechanism. A significant difference between the permeability coefficients in the two directions (efflux ratio of 1.5-2 or above), is a strong indication of active transport or efflux system involvement [17].

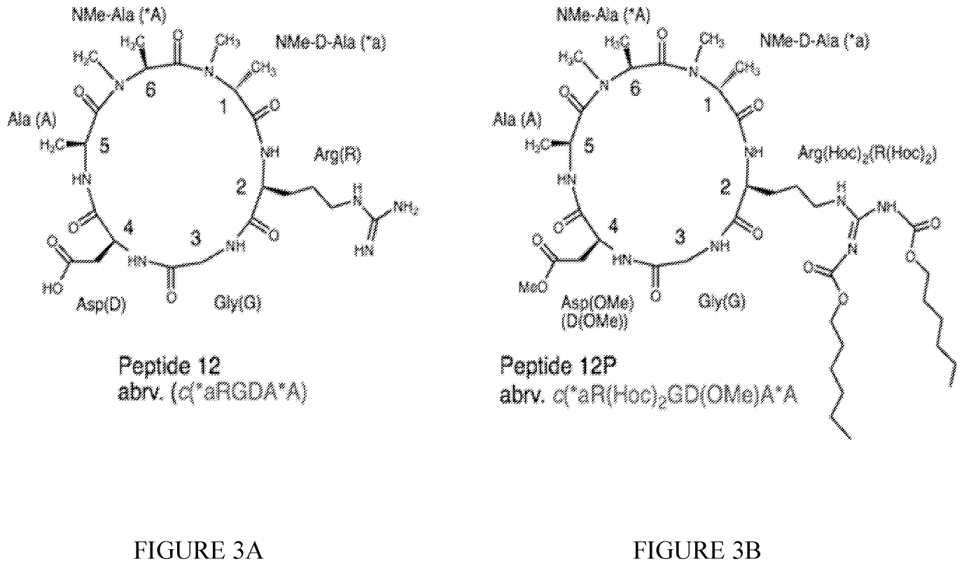

Peptide 12 (c(*aRGDA*A), called herein the “drug”, was selected from the RGD library (Peptides #5-28) because of its high affinity and selectivity to the integrin receptors. FIG. 4 presents the results of Caco-2 A-to-B assay of peptide 12 (c(*aRGDA*A)) and its prodrug peptide 12P(c(*aR(Hoc)₂GD(OMe)A*A)). The results show that charge masked prodrug have significantly increased permeability rate with Papp of 15.79 of the prodrug vs. 0.0617 of the drug.

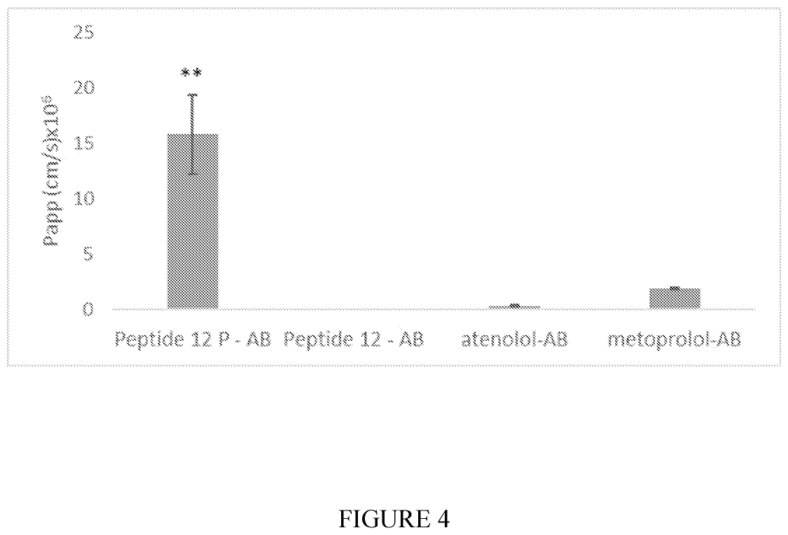

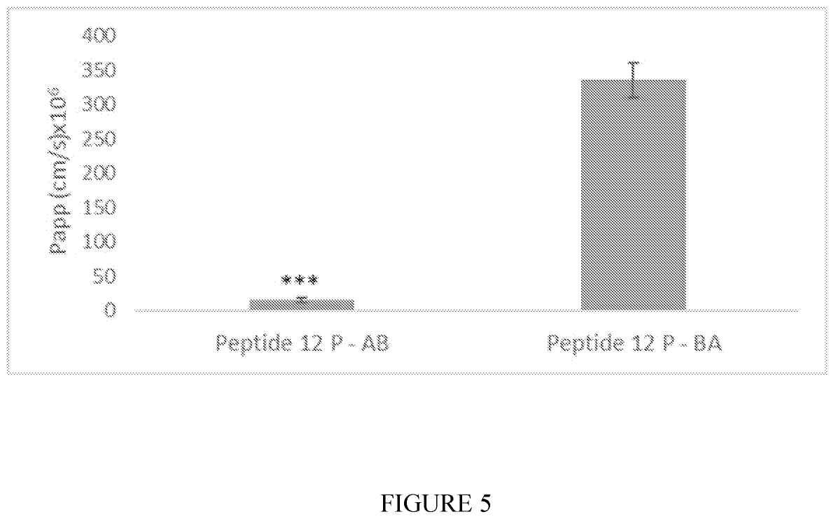

Furthermore, the B-to-A study, revealed higher Papp of peptide 12P than its A-to-B Papp (335.8 vs. 15.7, FIG. 5). The efflux ratio of peptide 12P is about 20. The efflux ratio of cyclosporine, a known P-gp substrate is 3. (FIG. 6). This ratio indicates significant involvement of efflux system in the permeability mechanism of 12P. Practically, any ratio higher than 2 is a valid indication of the involvement of the efflux activity.

It is important to note that the involvement of efflux system is actual indication that the prodrug is permeate through the enterocytes membrane and afterwards removed from these cells by the efflux system.

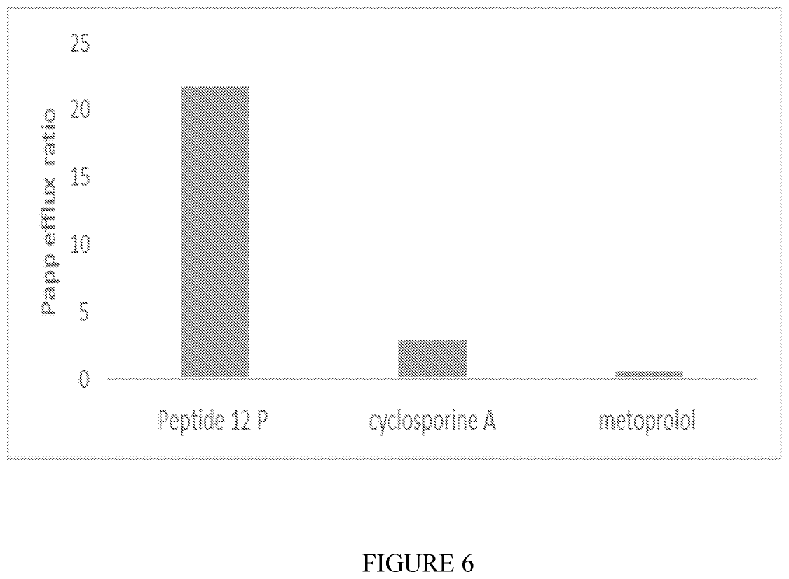

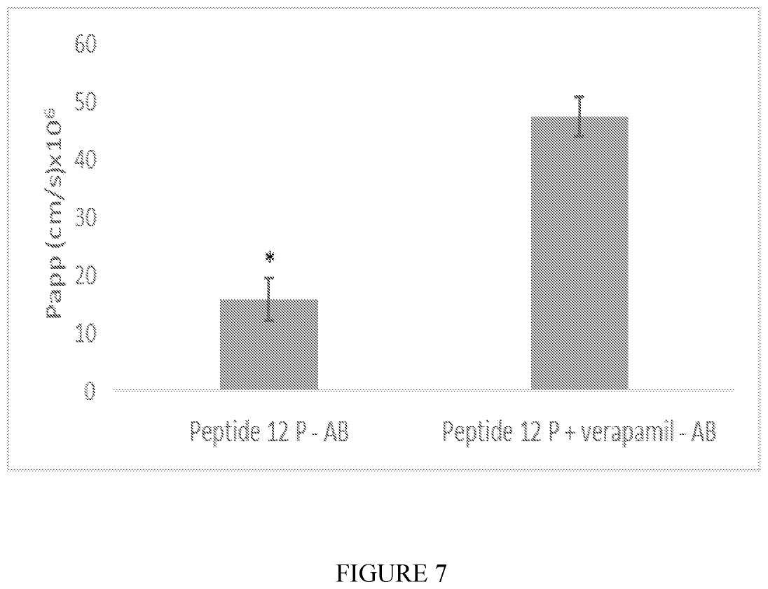

To further study the efflux system involved in the permeability mechanism of peptide 12P, a Caco-2 study in the presence of verapamil (100 mM), a known P-gp inhibitor was performed. The results (FIG. 7) show a 3-fold increase in Papp of peptide 12P, in the presence of verapamil, from 15.7 to 47.4. Prodrug peptide 12P was additionally tested in the presence of palmitoyl carnitine chloride (PC), which enhances the permeability of hydrophilic compounds by effecting the TJs of the epithelial barrier. FIG. 8 shows that the presence of PC affects the Papp values compared to verapamil, which is related to the inhibition of the efflux system. There is a significant difference between the Papp of peptide 12P alone (1.64±0.15 vs 12.52±0.20 cm/s×10⁶), whereas in the presence of PC, the AB and BA Papp values are similar (5.37±0.16 vs 6.80±0.28 cm/s×10⁶). This result further strengthens the hypothesis that peptide 12P permeates through the intestine monolayer with the involvement of the efflux systems.

### Example 3: Metabolic Stability Studies

Generally, the purpose of metabolic stability studies is to evaluate the compounds rate of elimination in the presence of hostile environments: a rat plasma or extractions of the gut wall. In these environments, compounds are prone to enzymatic degradation, as there are high concentrations of peptidases, esterases, lipases and other peptides that metabolize xenobiotics to building units for synthesizing essential structures in the body [18, 19].

Specifically, in our case, the purposes of the metabolic stability studies are (1) to prove that the prodrug (peptide 12P) is digested by esterases to furnish the drug (peptide 12) and (2) to demonstrate that peptides 12 and 12P are stable to digestion in the intestine.

The enzymatic reactions were performed as follows: 2 mM stock solutions of the tested compounds were diluted with serum or purified brush border membrane vesicles (BBMVs) solution to a final concentration of 0.5 mM. During incubation at 37° C. samples were taken for a period of 90 minutes. The enzymatic reaction was stopped by adding 1:1 v/v of ice cold acetonitrile and centrifuge (4000 g, 10 min) before analysis. Preparation of BBMVs: The BBMVs was prepared from combined duodenum, jejunum, and upper ileum (male Wistar rats) by a Ca++ precipitation method. Purification of the BBMVs was assayed using GGT, LAP and alkaline phosphatase as membrane enzyme markers

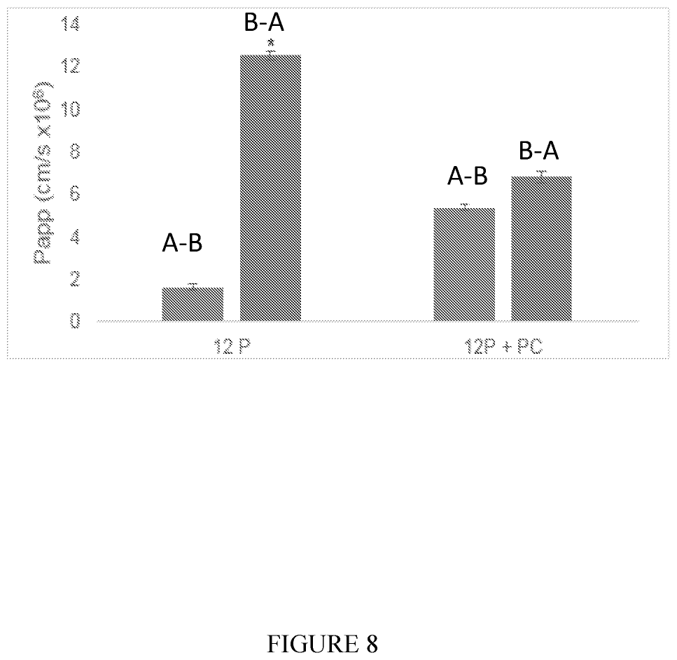

Peptides 12 and 12p were subjected to rat plasma and followed their degradation. Rat plasma is known to be rich with esterases. FIGS. 9A and 9B demonstrate the degradation of peptides 12 and 12P in rat plasma due to esterases activity. Peptide 12 remained stable during the incubation time, because it lacks ester bonds. Peptide 12P on the other hand is degraded to yield peptide 12 because it contains ester bonds (see FIG. 3B).

This experiment proves that peptide 12P is a prodrug of peptide 12.

Next, peptides 12 and 12P were subjected to extractions of the gut wall (brush border membrane vesicles, BBMV) and followed their rate of degradation. The BBMV assay determines the peptides stability in the presence of digestive enzymes in the brush border membrane of the intestine especially peptidases.

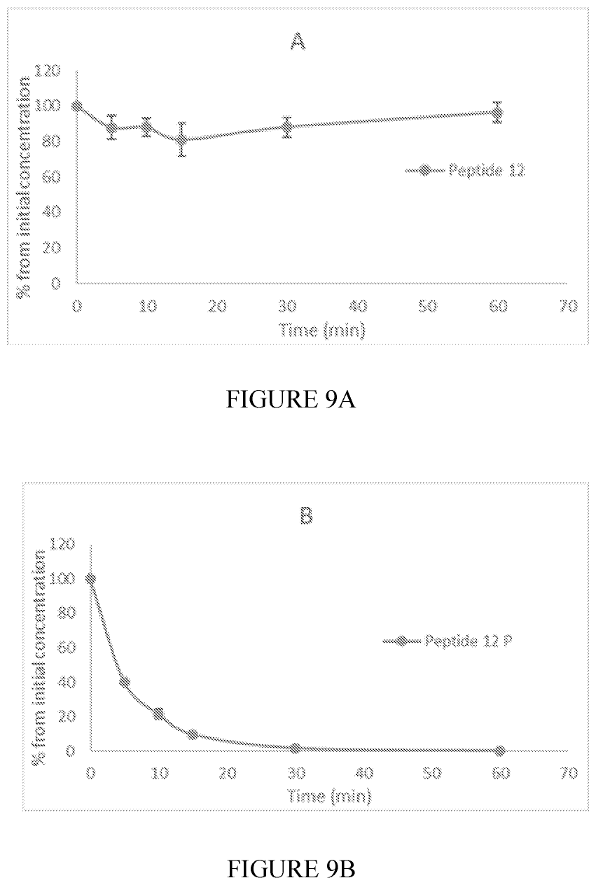

As can be seen from FIG. 10 both peptides are stable to enzymes in the BBMV which indicates oral bioavailability and therefore fulfill the DLP paradigm.

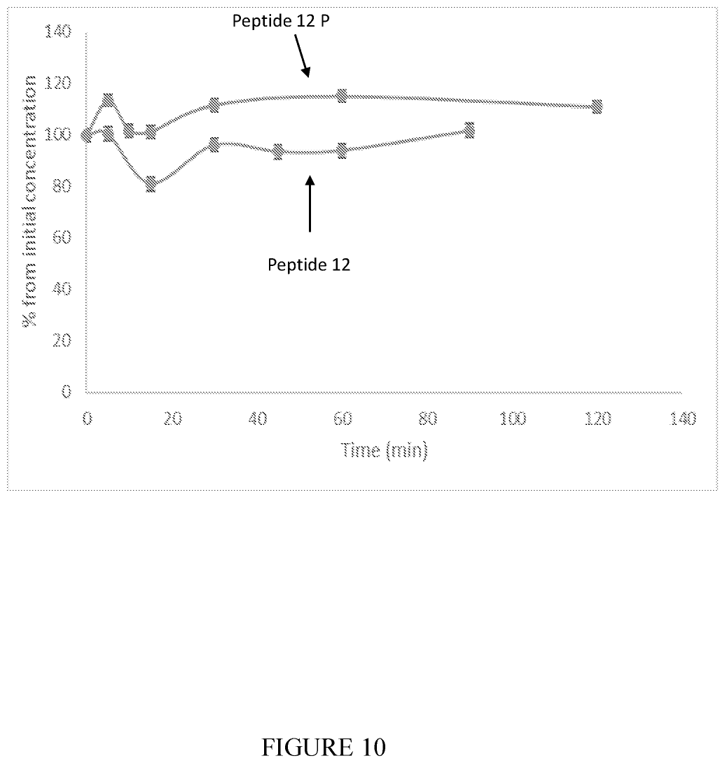

Peptide 12P was subjected to additional in vitro assay to evaluate the involvement of liver metabolism, through the Pooled Human Liver Microsome assay. Liver microsomes are subcellular particles derived from the endoplasmic reticulum of hepatic cells. These microsomes are a rich source of drug metabolizing enzymes, including cytochrome P-450. Microsome pools from various sources are useful in the study of xenobiotic metabolism and drug interactions. FIG. 11 presents the degradation of peptide 12P by Pooled Human Liver Microsomes. The presence of ketokonazole inhibits the metabolism by the liver enzymes in some degree. However, Incubating Peptide 12P with self-assembling pro-nano lipospheres (PNL), led to much better inhibition of cytochromes P-450. This result is another proof that Peptide 12P is a substrate for P-gp efflux system and cytochromes P-450, and while overcoming the permeability challenges, Peptide 12P formulation protection against efflux systems and enzymatic metabolism in the intestine and liver.

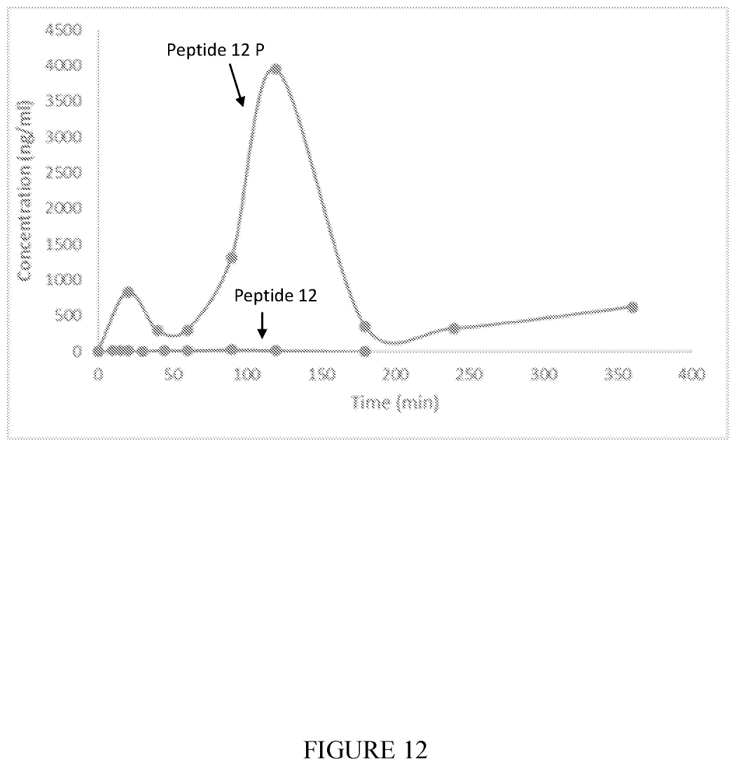

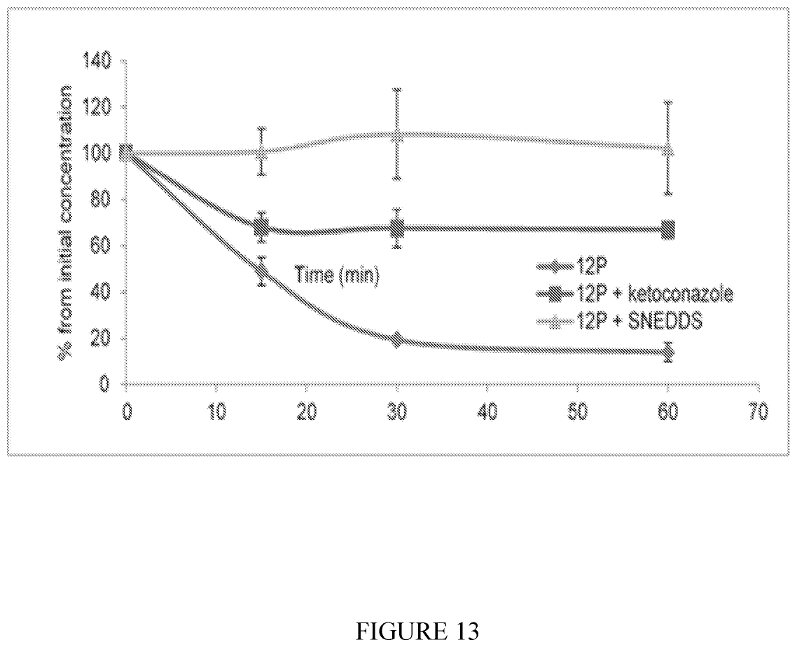

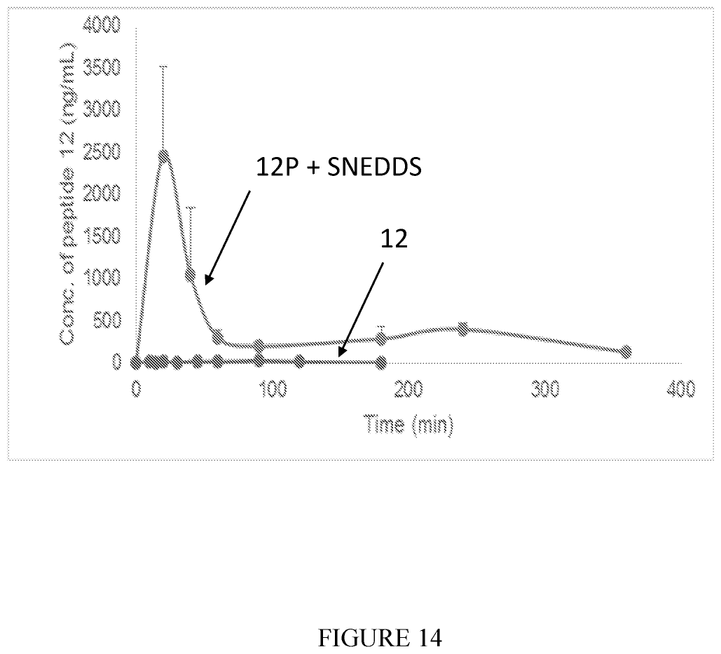

The mechanism of absorption was further tested in isolated rat CYP3A4 microsomes. The question of how the efflux is affected by ketoconazole, a specific CYP3A4 inhibitor, and by SNEDDS was also investigated. They were found to reduce CYP3A4 metabolism and reduce P-gp efflux (FIG. 13). The concentrations remaining following 60 min of incubation of dispersed peptide 12P were compared. The groups included peptide 12P with SNEDDS, 12P with ketoconazole, and 12P alone (102.2±19.7%, 67.0±3.61%, and 14.0±4.06% respectively). A significant difference (p<0.01) was found between peptide 12P and the dispersed 12P with SNEDDS and between 12P and 12P with ketoconazole (p<0.05). The plasma concentration-time profiles for peptide 12 and the dispersed 12P SNEDDS following oral administration of 5 mg/kg of peptides 12 or 12P to rats are shown in FIGS. 14 and 15. The corresponding AUC and Cmax parameters obtained in these in vivo experiments are listed in Table 2 and were significantly greater for the dispersed 12P SNEDDS in comparison to peptide 12. The relative bioavailability of peptide 12P was about 70-fold greater than that of peptide 12 after oral administration (FIG. 15).

TABLE 2
AUC, Cₘₐₓ, kₑ₁ values, and Tₘₐₓ values (Median
(range)) of Peptide 12 obtained following oral ad-
ministration of peptide 12 and dispersed 12P SNEDDS.

| | 12 | 12P |
|---|---|---|
| Cₘₐₓ (ng/ml) | 119 ± 86 | 1993 ± 967₁ |
| Tₘₐₓ (min | 45 (20-90) | 20 (20-60) |
| AUC (min*μg/ml) | 1.91 ± 0.37 | 216.9 ± 75.6 |
| kₑ₁ (min⁻¹) | 0.04 ± 0.005 | 0.009 ± 0.0001 |
| F(%) | 0.58 ± 0.11 | 43.8 ± 14.9 |

### Example 4: Pharmacokinetic Study

The pharmacokinetic in-vivo study allows a further evaluation of the prodrug concept in the whole animal. The PK studies were performed in conscious Wistar male rats. An indwelling cannula was implanted in the jugular vein 24 hours before the PK experiment to allow full recovery of the animals from the surgical procedure. Animals (n=4) received either an IV bolus dose or oral dose of the investigated compound. Blood samples (with heparin, 15 U/ml) were collected at several time points for up to 6 hours post administration and was assayed by HPLC-MS method. Non-compartmental pharmacokinetic analysis was performed using WinNonlin software.

This study showed significant increase in the area under the curve (AUC) of peptide 12 after peptide 12P administration. In other words, the PK study shows that after oral administration of peptide 12P (the prodrug), peptide 12 (the drug) appears in the systemic blood circulation. This proves that (a) peptide 12P is orally available (b) it is stable in the intestine, and (c) it is metabolized in the blood to regenerate peptide 12. To ensure good bioavailability of the drug, the prodrug was formulated in a nanoparticles formulation that is known to inhibit the P-gp efflux system. It should be mentioned that peptide 12 was also formulated in the same nanoparticle. In this case the formulation did not enhance oral bioavailability since this peptide is actually intestinally non-permeable (FIG. 4). These results are an in-vivo proof of concept for the prodrug approach.

Other peptides and their prodrug analogs were subjected to the Caco-2 assay and showed the same behavior as peptide 12.

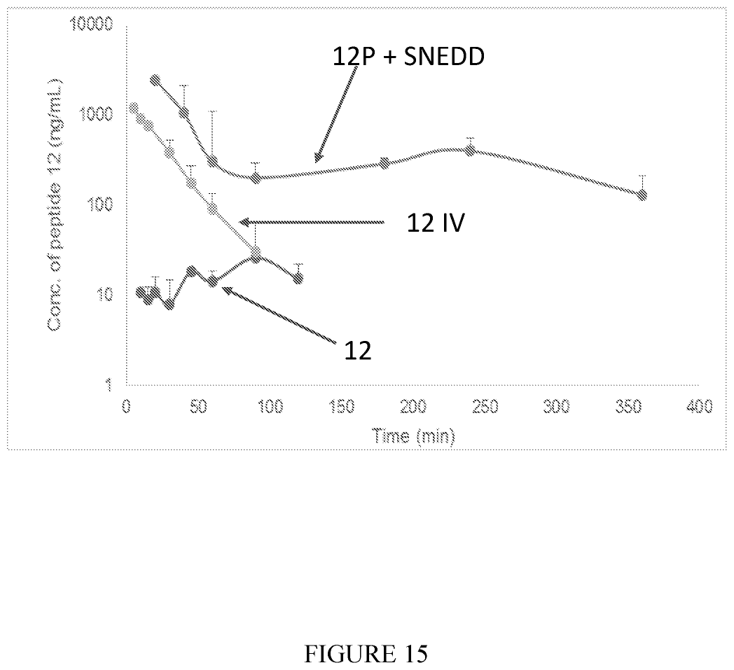

Peptide 29 (c(*vRGDA*A) and its prodrug 29P (c(*vR(Hoc)₂GD(OMe)A*A)) were selected from the RGD library (Peptides #5-28, FIG. 2A) for further proof of concept because of its high affinity and selectivity to the integrin receptors. The structures of both peptides are shown in FIGS. 16A and 16B.

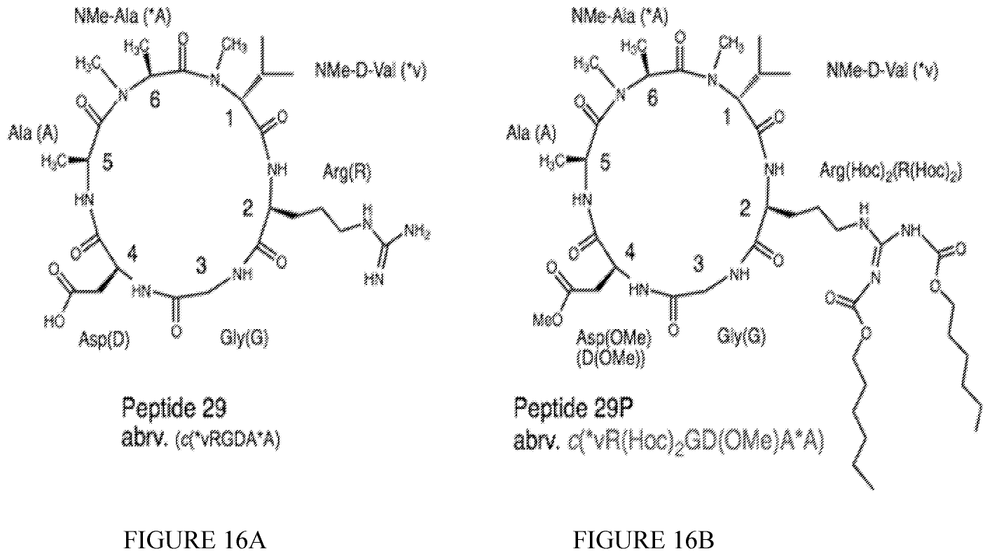

The permeability of both peptides (peptide 29 and 29P) is low. The Papp of Peptide 29P is lower in the A-to-B assay, than the Papp of Peptide 29 (0.08 vs. 0.6 respectively), as shown in FIG. 17.

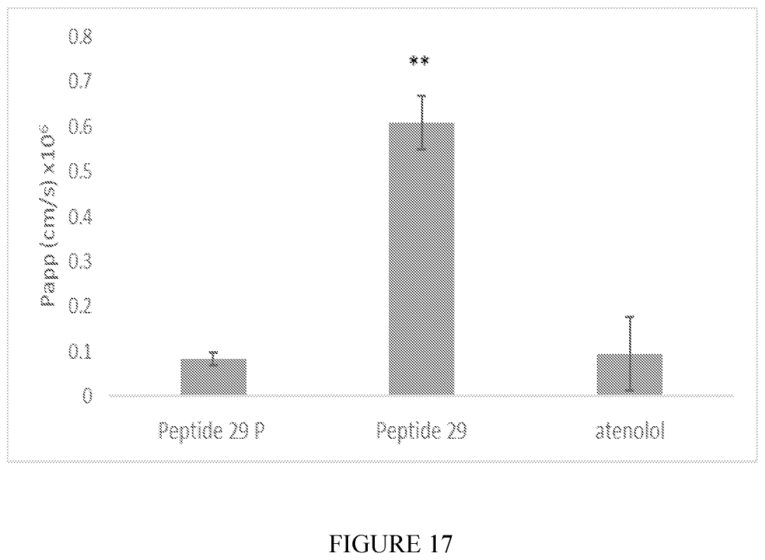

This unanticipated result is clarified when comparing the B-to-A Papp of Peptide 29P to its A-to-B Papp (FIG. 18). The B-to-A Papp of the prodrug is significantly higher than the A-to-B Papp (0.08 vs. 1.06), suggesting that the low A-to-B Papp was resulted from extensive activity of the efflux system.

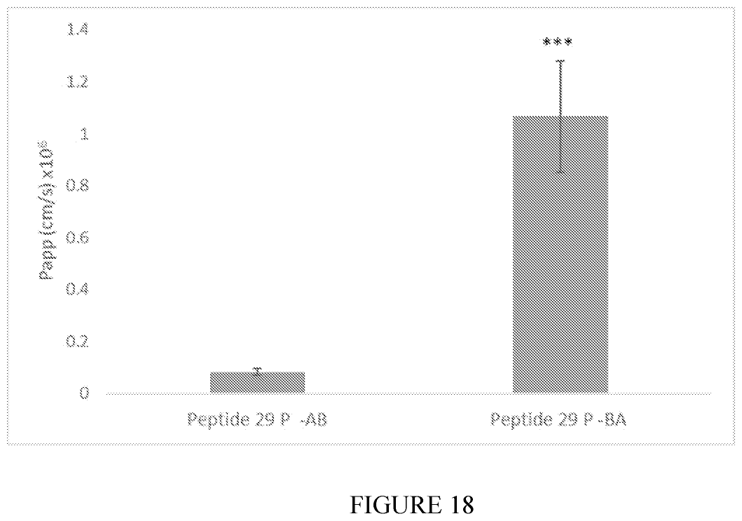

Peptide 5 (c(*rGDA*AA, SEQ ID NO: 1) and its prodrug, peptide 5P (c(*r(Hoc)₂GD(OMe)A*AA, SEQ ID NO: 11) were also evaluated. In these peptides, the N-methylation pattern is 1,5 rather than 1,6 (the pattern in peptide 29 and its prodrugs). Also in these peptides (5 and 5P) the D-amino acid is Arginine. In the Caco-2 model, both the drug (peptide 5) and the prodrug (peptide 5P) exhibit relatively low Papps (0.03 and 0.06) which is very similar to the atenolol Papp (0.025, FIG. 19).

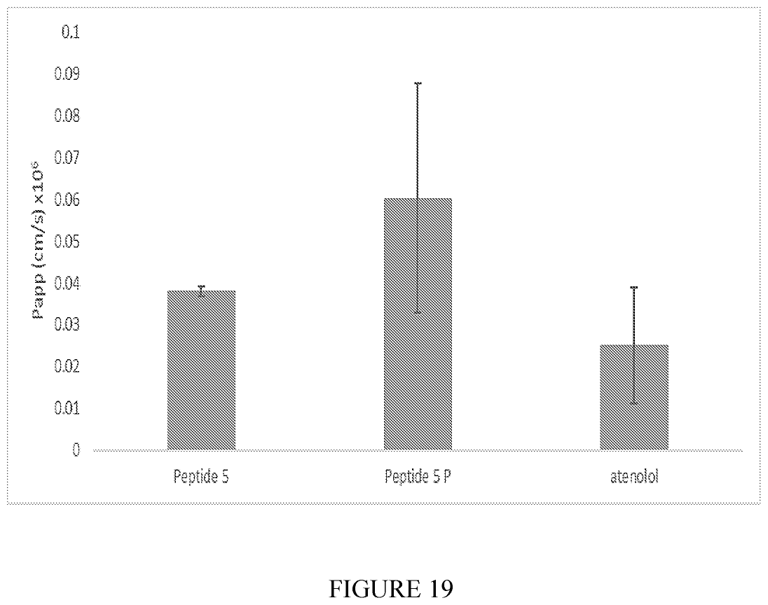

The B-to-A permeability of peptide 5P resulted in much higher Papp than its A-to-B Papp (2.12 vs 0.06, FIG. 20), suggesting again the involvement of efflux system, which attributes to peptide 5P's low A-to-B permeability of the prodrug.

Peptides 17, 23, and 30 and their corresponding prodrugs 17P (SEQ ID No: 13), 23P (SEQ ID No: 12), and 30P (SEQ ID No: 14) show the same pattern of intestinal permeability as peptides 12 and 12P, 29 and 29P and 5 and 5P. Table 3 Summarizes Papp efflux A-B and B-A of the examined RGD peptides.

TABLE 3
Pₐₚₚ values (n = 3 for each group) of RGD peptides and their prodrug derivatives
for AB and BA permeability and the efflux ratio in Caco-2 cell model.

| | | | Pₐₚₚ AB | Pₐₚₚ BA | efflux |
|---|---|---|---|---|---|
| scaffold | peptide | Sequence | [cm/s × 10⁶] | [cm/s × 10⁶] | ratio |
| NMe(1, 5) | 5 | c(*rGDA*AA) | 0.38 ± 0.01 | 0.42 ± 0.11 | 1.1 |
| NMe(1, 5) | 5P | c(*r(Hoc)₂GD(OMe)A*AA) | 0.6 ± 0.27 | 22.12 ± 3.58 | 36.73 |
| NMe(1, 6) | 12 | c(*aRGDA*A) | 0.04 ± 0.02 | 0.12 ± 0.01 | 2.80 |
| NMe(1, 6) | 12P | c(*aR(Hoc)₂GD(OMe)A*A) | 0.79 ± 0.18 | 16.8 ± 1.3 | 12.76 |
| NMe(5, 6) | 23 | c(rGDA*A*A) | 0.61 ± 0.09 | 1.34 ± 0.03 | 2.19 |
| NMe(5, 6) | 23P | c(r(Hoc)₂GD(OMe)A*A*A) | 1.77 ± 0.55 | 74.77 ± 20.36 | 42.24 |
| NMe(1, 6) | 29 | c(*vRGDA*A) | 0.07 ± 0.01 | 0.15 ± 0.01 | 2.14 |
| NMe(1, 6) | 29P | c(*vR(Hoc)₂GD(OMe)A*A) | 0.82 ± 0.13 | 10.66 ± 2.14 | 13 |
| | atenolol^{a} | | 0.31 ± 0.08 | | |
| | metoprolol^{b} | | 1.89 ± 0.11 | | |

^{a}atenolol is a marker for paracellular permeability,
^{b}metoprolol is a marker for transcellular permeability.

Previous work has shown that Cilengitide has the potential to have anti-angiogenic effects. Unfortunately however, clinical trials using this drug in the treatment of glioblastoma were disappointing and production of this drug has been discontinued. We have published that actually low doses of Cilengitide can have vascular promotion effects, i.e. increasing tumour angiogenesis above and beyond that of the untreated tumor [20]. Indeed, we have evidence that in combination with the appropriate chemotherapeutics vascular promotion induced by treatment with low dose Cilengitide is sufficient to halt tumor growth in pre-clinical mouse models of cancer [15]. This provides an exciting opportunity to exploit vascular promotion in combination with chemotherapy or indeed other therapies where increasing delivery to the tumor might be of benefit. The prodrug approach presented here is a potential to exceed the Cilengtide efficacy.

### Example 5: Molecular Docking Methods

The crystal structures of αvβ3 (PDB code: 1L5G)[21] in complex with Cilengetide was prepared for docking calculations using the Protein Preparation Wizard tool of the Schrödinger 2016 molecular modeling package [22]. First, the Mn2+ ion at the MIDAS was replaced with Mg2+. Next, all the bond orders were assigned, the disulfide bonds were created and all the hydrogen atoms were added; the prediction of the side chains hetero groups ionization and tautomeric states was performed using Epik 3.7.[23, 24] Finally, an optimization of the hydrogen-bonding network and of the hydrogen atoms positions was performed using the ProtAssing and impref utilities, respectively. All water molecules were deleted prior to docking calculations. Docking studies were carried out with the grid-based program Glide v. 7.2 [25,26]. For the grid generation, a virtual box of 20 Å×20 Å×20 Å surrounding the ligand RGD binding cavity was created. The standard precision mode for peptide ligands (SP-peptide) and the OPLS3 force field [27] were chosen to run calculations and to score the predicted binding poses. The lowest energy solution (docking scores: −7.433) that could properly recapitulate the typical RGD interaction pattern was selected for the binding mode description. All of the pictures were rendered with PyMOL.

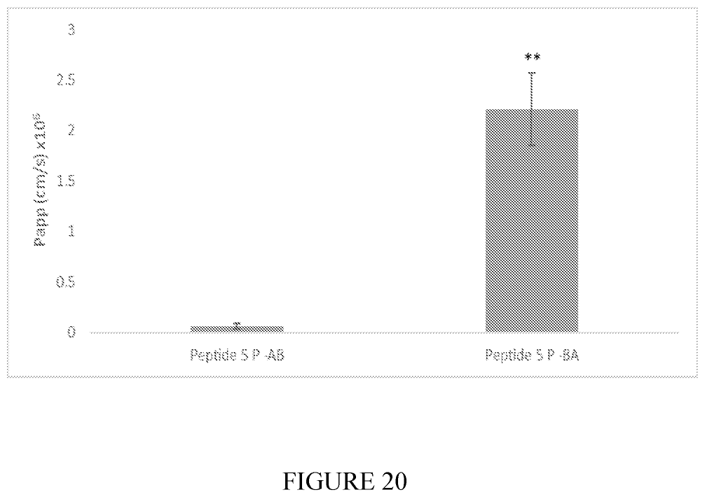

To describe at an atomic level the binding mode of the cyclic hexapeptides to integrin receptors, the solution state structure of 29 was calculated by NMR studies (FIG. 21A) and was used for performing docking calculations of 29 at the αvβ3 RGD binding site. According to the docking results, 29 binds to αvβ3 (FIG. 21B) very similarly to the reference ligand Cilengitide. In detail, the Asp³ carboxylate group coordinates the metal ion at the MIDAS and forms two H-bonds (β3)-Asn215, while the NMe-d-Arg′ guanidinium group establishes a tight salt bridge with the (αv)-Asp218 side chain and a cation-π with the (αv)-Tyr178 phenolic ring. The 29/αvβ3 complex is further stabilized by an additional H-bond between the Asp⁴ backbone CO and the (β3)-Arg214 side chain and by lipophilic contacts between NMe-Ala⁶ and (β3)-Met180. The predicted binding mode is thus overall consistent with the subnanomolar IC₅₀ observed for 29 at the αvβ3 receptor.

### Example 6: Comparing Peptide 29 and 29P Derivatives to Control Molecules

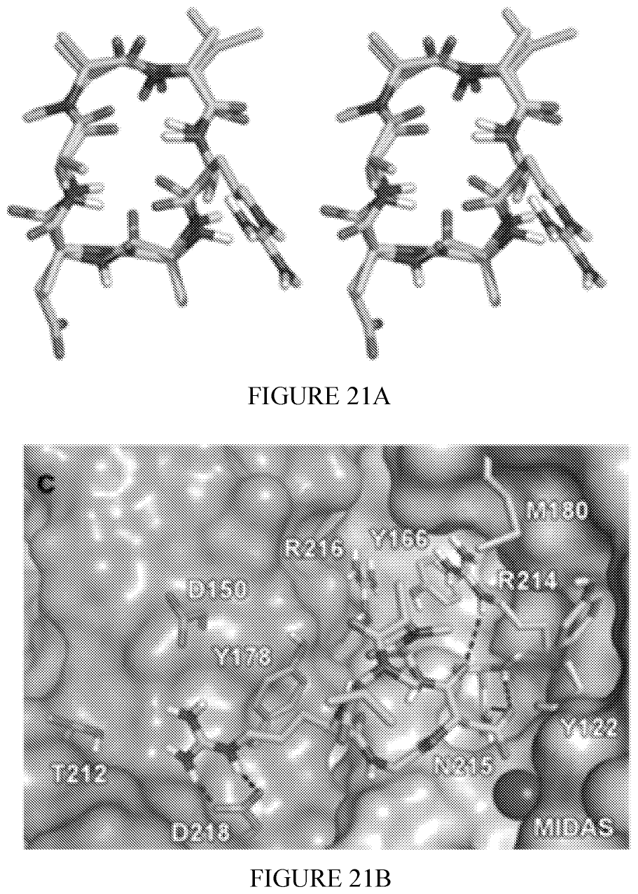

The RGD cyclohexapaptides library was further investigated for its physicochemical properties in vitro, using Log D, caco-2 and PAMPA models. The investigated peptide derivatives are depicted in FIG. 22.

Log D. Determination of distribution coefficients were performed as follows:

Incubations were carried out in Eppendorf-type polypropylene microtubes in triplicates. 5 μL aliquot of compound DMSO stock (10 mM) was dissolved in the previously mutually saturated mixture containing 500 μL of PBS (pH 7.4) and 500 μL of octanol followed by mixing in a rotator for 1 hour at 30 rpm. Phase separation was assured by centrifugation for 2 min at 6000 rpm. The octanol phase was diluted 100-fold with 40% acetonitrile, and aqueous phase was analyzed without dilution. The samples (both phases) were analyzed using HPLC system coupled with tandem mass spectrometer. Mebendazole was used as a reference compound (experimental log D, pH 7.4 range is 2.9-3.15). The log D values depicted in Table 2 show that the addition of lipophilic residues to the peptides, elevate the log D value, indicating higher distribution in the lipophilic phase and environment. This is evident for peptide 29 (#29), peptide 29P having a single Hoc (#29P-Hoc; (SEQ ID NO: 21)), and peptide 29P having 2 Hoc molecules (#29P). The results show that with no lipophilic residues (#29), the log D is <−1. Adding one Hoc and OMe group (#29P-Hoc) elevate the log D value to 1.85, and the completely protected peptide (#29P) has the highest value of 4.86. Similar results are seen when comparing the log D values of other peptides and their prodrug derivatives in Table 4.

TABLE 4
Log D values for the cyclic peptides, in comparison to mebendazole

| Compound ID | LogD, pH 7.4 | | Compound ID | LogD, pH 7.4 | |
|---|---|---|---|---|---|
| Mebendazole | 3.02 | 3.04 | Cilengitide | −1.52 | <−1 |
| | 3.05 | | | −1.75 | (−1.68) |
| | 3.03 | | | −1.79 | |
| #29P | 5.07 | 4.86 | AR372 | 5.02 | >4.5 |
| | 4.80 | | | 4.72 | (4.85) |
| | 4.72 | | | 4.80 | |
| #29 | −5.01 | <−1 | OM1186 | −0.22 | −0.19 |
| | −1.72 | (−2.84) | | −0.21 | |
| | −1.80 | | | −0.14 | |
| #29P-HOC | 1.91 | 1.85 | AR373 | 5.17 | >4.5 |
| | 1.87 | | | 5.22 | (5.21) |
| | 1.78 | | | 5.25 | |
| #29P* | 4.95 | >4.5 | FRX068 | −1.33 | <−1 |
| | 4.97 | (4.95) | | −1.55 | (−1.49) |
| | 4.94 | | | −1.58 | |
| 1,6CHA | −0.50 | −0.97 | | | |
| | −1.14 | | | | |
| | −1.26 | | | | |
| Cil-P | 3.76 | 3.95 | | | |
| | 4.00 | | | | |
| | 4.10 | | | | |

PAMPA.

The Parallel Artificial Membrane Permeability Assay (PAMPA) is used as an in vitro model of passive, transcellular permeation. PAMPA eliminates the added complexities of active transport, allowing ranking compounds just based on a simple membrane permeability property. This assay also allows evaluation of permeability over a large pH range, which is valuable for a preliminary understanding of how orally delivered compounds might be absorbed across the entire gastrointestinal tract. PAMPA was first introduced by Kansy et al. and has been since widely used in the pharmaceutical industry as a high throughput, quick and inexpensive permeability assay to roughly evaluate oral absorption potential. Depending upon the types of lipids used and other experimental conditions, PAMPA may be designed to model absorption in gastrointestinal tract (PAMPA-GIT), blood-brain barrier penetration (PAMPA-BBB) or skin penetration (Skin PAMPA). All steps of the PAMPA were carried out according to pION Inc. PAMPA Explorer™ Manual. The main principle of the assay is the incubation of compound in donor chamber (a well in Donor Plate) with aqueous buffer, which is separated from acceptor chamber (a well in Acceptor Plate) with another buffer by a phospholipid or hydrocarbon membrane fixed on a filter support. After the test, concentrations in the corresponding donor and acceptor wells are measured and permeability is calculated. GIT model was simulated using GIT-0 phospholipid mix. Verapamil and quinidine (high permeability) and ranitidine (low permeability) were used as reference compounds. All compounds were tested in triplicates. Prisma HT buffer (pH 7.4) containing 50 μM test compounds and 0.5% DMSO were added into the Donor Plate wells. Acceptor Sink buffer was added into each well of the acceptor plate. Incubation was done at room temperature for 4 hours without stirring. After incubation, aliquots from both plates were transferred to optic UV-Vis plates and optic plates were read on microplate reader in absorbance mode in the range of 102-500 nm with 4 nm step. Compounds with low UV-Vis signal were detected by LC-MS/MS method. Then the apparent permeability coefficient was calculated. Results are shown in Table 5.

TABLE 5
PAMPA permeability coefficients of the peptide library, in comparison to quinidine,
verapamil and ranitidine.

| | Permeability, Log₁₀[10⁻⁶ cm/s] | | | | | |
|---|---|---|---|---|---|---|
| Compound ID | 1 | 2 | 3 | Mean | SD | Mass retention, % |
| Quinidine | −4.6 | −4.5 | −4.5 | −4.5* | 0.06 | 56 |
| Verapamil | −4.1 | −4.0 | −4.3 | −4.2* | 0.16 | 44 |
| Ranitidine | <−7 | <−7 | <−7 | <−7* | — | 21 |
| #29P | −4.9 | −4.3 | −3.9 | −4.4 | 0.48 | 35 |
| #29 | <−7 | <−7 | <−7 | <−7 | — | 15 |
| #29P-Hoc | <−7 | <−7 | <−7 | <−7 | — | 10 |
| #29P* | −4.3 | −4.3 | −4.3 | −4.3 | 0.00 | 20 |
| 1,6CHA | <−7 | <−7 | <−7 | <−7 | — | 27 |
| Cil.-p | Outlier | −6.6 | −6.5 | −6.5 | 0.92 | 36 |
| | −8.1 | | | | | |
| Cilengitide | <−7 | <−7 | <−7 | <−7 | — | 1 |
| AR372 | −6.2 | −5.7 | −5.7 | −5.9 | 0.27 | 80 |
| OM1186 | <−7 | <−7 | <−7 | <−7 | — | 13 |
| AR373 | −5.6 | −5.1 | −4.8 | −5.2 | 0.43 | 22 |
| FRX068 | <−7 | <−7 | <−7 | <−7 | — | 2 |

*The compounds’ structure is shown in Fig. 22. #29P is SEQ ID NO: 9, #29 is SEQ ID NO: 5, #29P-Hoc is SEQ ID NO: 21, #29P* is enantiomer of 29P (SEQ ID NO: 9), Cil.-P is pro drug of Cilengitide (c(f*VR(Hoc)₂ GD); SEQ ID NO: 20), and 1,6CHA is SEQ ID NO: 22 (*aAAAA*A).

Peptides 29P (#29P) and #29P* (enantiomers) showed high permeability (>−5) in the PAMPA-GIT model system. Permeability of the two test compounds (AR372 (SEQ ID NO: 15) and AR373 (SEQ ID NO: 16)) was in the range of >−5 to >−6. These results strengthen the hypothesis that LPCM enhances the permeability of RGD cyclohexapeptides through lipophilic membranes. Evidently, #29 (the unprotected derivative) show low permeability (<−7) and interestingly, the semi-protected #29P-Hoc also exhibits low permeability in PAMPA, suggesting that fully protected peptide is more permeable.

Cilengitide is a cyclopentapeptide with one N-methylated group (other peptides tested are cyclohexapeptides, with two N-methylated groups). It shows low permeability in PAMPA, however, LPCM protection (Cil.-P; SEQ ID NO: 23) does not enhance the permeability, and this suggests that there are also structural considerations that influence the permeability, other than the lipophilicity of the peptide (log D of Cil.-P is 3.95, vs. <−1 in Cilengitide).

Caco-2. Caco-2 cells were cultured in 75 cm2 flasks to 80-90% confluence according to the ATCC and Millipore recommendations. in humidified atmosphere at 37° C. and 5% CO₂. Cells were detached with Trypsin/EDTA solution and resuspended in the cell culture medium to a final concentration of 2×10⁵ cells/ml. 500 μl of the cell suspension was added to each well of HTS 24-Multiwell Insert System and 1000 μl of prewarmed complete medium was added to each well of the feeder-plate. Caco-2 cells were incubated in Multiwell Insert System for 21 days before the transport experiments. The medium in filter plate and feeder tray was refreshed every other day. After 21 days of the cell growth, the integrity of the monolayer was verified by measuring the transepithelial electrical resistance (TEER) for every well using the Millicell-ERS system ohm meter. The final TEER values were within the range 150-600 Ω×cm2 (Srinivasan B. et al., 2015) as required for the assay conditions. 24-well insert plate was removed from its feeder plate and placed in a new sterile 24-well transport analysis plate. The inserts were washed with PBS after medium aspiration. Propranolol, Atenolol, Quinidine and Digoxin were used as reference compounds. To determine the rate of compounds transport in apical (A)-to-basolateral (B) direction, 300 μL of the test compound dissolved in transport buffer at 10 μM (MSS, 25 mM HEPES, pH=7.4) was added into the filter wells; 1000 μL of buffer (MSS, 25 mM HEPES, pH=7.4) was added to transport analysis plate wells. To determine transport rates in the basolateral (B)-to-apical (A) direction, 1000 μL of the test compound solutions was added into the wells of the transport analysis plate, the wells in filter plate were filled with 300 μL of buffer (apical compartment). The final concentrations of the test compounds were 10 μM. The effect of the inhibitor on the P-gp-mediated transport of the tested compounds was assessed by determining the bidirectional transport in the presence or absence of verapamil. The Caco-2 cells were preincubated for 30 min at 37° C. with 100 μM of verapamil in both apical and basolateral compartments. After removal of the preincubation medium the test compounds (final concentration 10 μM) with verapamil (100 μM) in transport buffer were added in donor wells, while the receiver wells were filled with the appropriate volume of transport buffer with 100 μM of verapamil. The plates were incubated for 90 min at 37° C. under continuous shaking at 50 rpm. 75 μL aliquots were taken from the donor and receiver compartments for LC-MS/MS analysis. All samples were mixed with 2 volumes of acetonitrile followed by protein sedimentation by centrifuging at 10000 rpm for 10 minutes. Supernatants were analyzed using the HPLC system coupled with tandem mass spectrometer. Results are shown in Tables 7 and 8.

TABLE 7
A-B and B-A permeability data

| | Pₐₚₚ (AB), 10⁻⁶ cm/s | | | | | Pₐₚₚ (BA), 10⁻⁶ cm/s | | | | | Net |
|---|---|---|---|---|---|---|---|---|---|---|---|
| Test compound | 1 | 2 | 3 | Mean | SD | 1 | 2 | 3 | Mean | SD | efflux* |
| Atenolol | 1.0 | 0.8 | 0.4 | 0.8 | 0.3 | | | | | | |
| Propranolol | 14.9 | 24.1 | 15.3 | 18.1 | 5.2 | 13.6 | 15.3 | 17.0 | 15.3 | 1.7 | 0.8 |
| Digoxin | 0.4 | 0.3 | 0.5 | 0.4 | 0.1 | 9.4 | 12.0 | 14.7 | 12.0 | 2.6 | 28.6 |
| Quinidine | 6.2 | 4.4 | 3.9 | 4.8 | 1.2 | 18.8 | 25.0 | 27.1 | 23.6 | 4.3 | 4.9 |
| #29P | 0.1 | 0.1 | 0.1 | 0.1 | 0.1 | 15.7 | 17.9 | 16.4 | 16.7 | 1.1 | 144.4 |
| #29 | 1.0 | 0.2 | 0.9 | 0.7 | 0.4 | 0.3 | 0.4 | 0.2 | 0.3 | 0.1 | 0.4 |
| #29P-Hoc | 0.3 | 0.3 | 0.2 | 0.3 | 0.0 | 0.2 | 0.2 | 0.3 | 0.3 | 0.1 | 1.0 |
| 1,6CHA | 0.0 | 0.1 | 0.1 | 0.1 | 0.1 | 0.1 | 0.1 | 0.2 | 0.1 | 0.1 | 1.1 |
| #29P | 0.4 | 0.4 | 0.2 | 0.3 | 0.1 | 17.7 | 20.8 | 21.4 | 20.0 | 2.0 | 62.9 |
| Cil.-P | 0.6 | 0.2 | 0.2 | 0.3 | 0.2 | 0.4 | 0.3 | 0.3 | 0.3 | 0.0 | 1.1 |
| Cilengititle | 0.4 | 0.6 | 0.6 | 0.6 | 0.1 | 0.6 | 0.8 | 0.5 | 0.6 | 0 1 | 1.1 |
| AR372 | <0.01** | <0.01** | <0.01** | <0.01 | — | 0.5 | 0.5 | 0.3 | 0.4 | 0.1 | 40 |
| OM1186 | <0.6** | <0.3** | — | <0.3 | — | 0.1 | 0.2 | 0.3 | 0.2 | 0.1 | 0.6 |
| AR373 | <0.1** | — | <0.1** | <0.1 | — | 8.6 | 12.1 | 10.9 | 10.6 | 1.8 | 152.7 |
| FRX068 | 0.3 | 0.2 | 0.2 | 0.3 | 0.1 | 0.3 | 0.3 | 0.3 | 0.3 | 0.0 | 1.1 |

*Efflux ratio is expressed as the quotient of Pₐₚₚ (BA) to Pₐₚₚ (AB)
**The obtamed experimental value for receiver compartment is less than LOD (3 × [signal-to-noise] value for a compound

TABLE 8
Data of A-B and B-A permeability in the presence of Verapamil

| | Pₐₚₚ (AB), 10⁻⁶ cm/s | | | | | Pₐₚₚ (BA), 10⁻⁶ cm/s | | | | | Net |
|---|---|---|---|---|---|---|---|---|---|---|---|
| Test compound | 1 | 2 | 3 | Mean | SD | 1 | 2 | 3 | Mean | SD | efflux* |
| Digoxin | 2.4 | 2.8 | 3.2 | 2.8 | 0.4 | 5.0 | 4.4 | 3.6 | 4.3 | 0.7 | 1.5 |
| Quinidine | 24.8 | 20.9 | 22.5 | 22.7 | 2.0 | 13.8 | 1.1 | 19.7 | 16.9 | 3.0 | 0.7 |
| #29P | 1.6 | 1.9 | 2.0 | 1.8 | 0.2 | 11.7 | 13.1 | 14.1 | 13.0 | 1.2 | 7.1 |
| #29 | <0.8** | — | — | <0.8 | — | 0.4 | 0.2 | 0.3 | 0.3 | 0.1 | 0.1 |
| #29P-Hoc | 0.3 | 0.3 | 0.2 | 0.3 | 0.1 | 0.1 | 0.1 | 0.1 | 0.1 | 0.0 | 0.5 |
| 1,6CHA | 1.6 | 0.3 | 0.1 | 0.7 | 0.8 | 0.1 | 0.1 | 0.1 | 0.1 | 0.0 | 0.2 |
| #29P* | 1.8 | 1.3 | 1.1 | 1.4 | 0.4 | 12.7 | 14.4 | 14.4 | 13.9 | 1.0 | 9.8 |
| Cil.-P | 1.0 | 0.7 | 0.5 | 0.7 | 0.2 | 0.3 | 0.2 | 0.1 | 0.2 | 0.1 | 0.2 |
| Cilengititle | 0.2 | 0.2 | 0.2 | 0.2 | 0.0 | 0.3 | 0.3 | 0.4 | 0.4 | 0.0 | 1.6 |
| AR372 | 0.7 | 0.3 | 0.3 | 0.4 | 0.2 | 0.2 | 0.2 | 0.2 | 0.2 | 0.0 | 0.5 |
| OM1186 | 0.2 | 0.0 | 0.5 | 0.2 | 0.3 | 0.2 | 0.2 | 0.2 | 0.2 | 0.0 | 0.7 |
| AR373 | 0.8 | 0.6 | 0.4 | 0.6 | 0.2 | 4.2 | 4.5 | 5.3 | 4.7 | 0.6 | 7.7 |
| FRX068 | 0.6 | 0.1 | 0.2 | 0.3 | 0.2 | 0.4 | 0.4 | 0.2 | 0.3 | 0.1 | 1.1 |

*Efflux ratio is expressed as the quotient of Pₐₚₚ (BA) to Pₐₚₚ (AB)
**The obtained experimental value for receiver compartment is less than LOD (3 × [signal-to-noise] value for a compound

#29P and #29P* (enantiomer) showed high permeability, while #29P-Hoc showed lower permeability in PAMPA. This is compatible with the caco-2 results—the LPCM method enhances the permeability through the lipophilic membrane, and low permeability in caco-2 (AB) is due to efflux activity. In past caco-2 results, only two Hoc groups protection or only OMe protection (in peptide 12) also was not enough to significantly enhance permeability. It seems that all three protection groups better enhance the permeability. AR372 (SEQ ID NO: 15) and AR373 (SEQ ID NO: 16) are prodrugs for OM1186 (SEQ ID NO: 17) and FRX068 (SEQ ID NO: 18), respectively. The caco-2 and PAMPA results of these peptides are compatible with the RGD library. In the presence of verapamil, the efflux ratio is lower significantly in these peptides.

Prodrug modification for Cilengitide did not enhance the permeability in Caco-2 and does not show efflux activity, which was typical for other RGD prodrug derivatives. The LPCM does not seem to work here, since it does not elevate the permeability in caco-2 or PAMPA and does not show efflux activity.

### Example 7: In Vivo Study

In Vivo Study

To estimate the efficacy of the peptides in inhibition of human cancer, the peptides are studied in tumor mice models. Mice are challenged with human cancer cells and treated with increasing concentrations of the prodrugs described herein above. The peptides are administered orally and compared to controls.

### Example 8: Preparation of Octreotide Prodrug

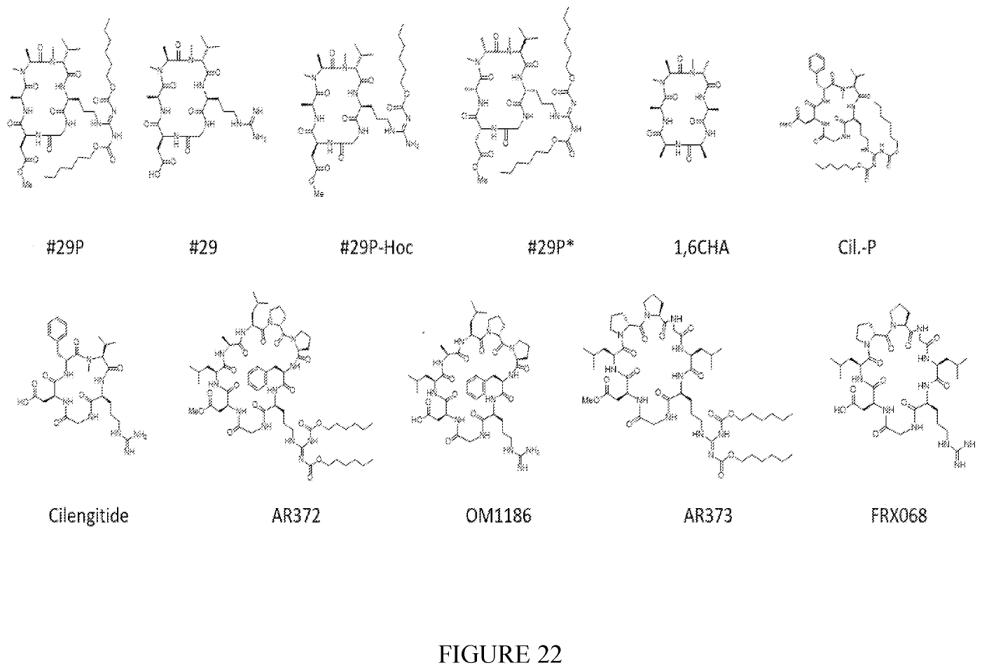

The prodrug hexyloxycarbonyl octreotide (Octreotide-P) was synthesized from octreotide using the synthetic pathway shown in FIG. 23.

### Example 9. Synthesis of Somato 8 Prodrug

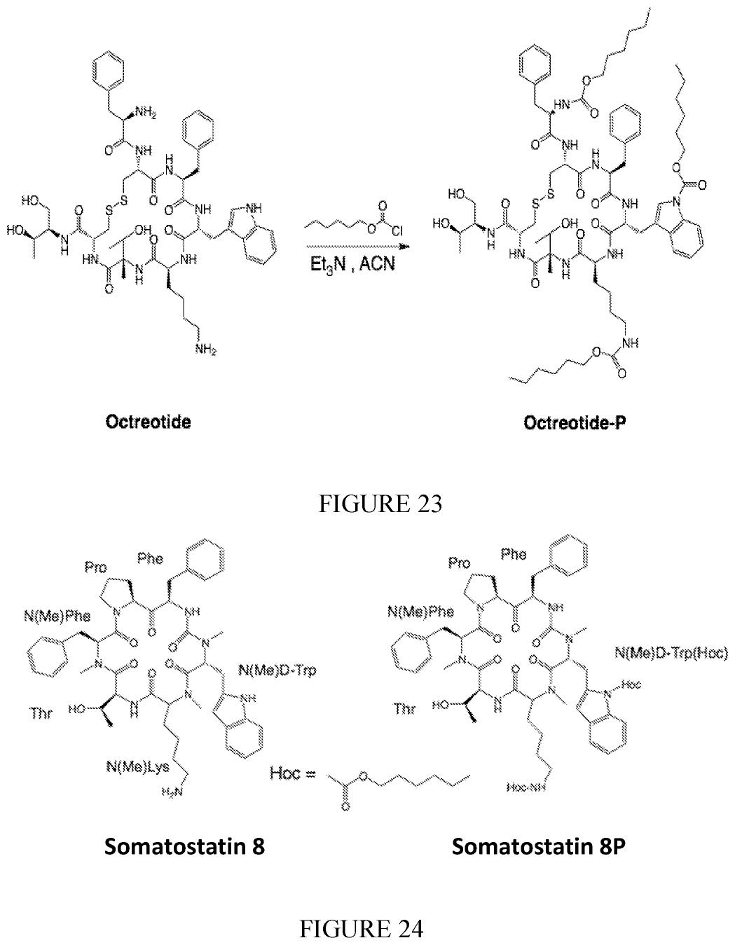

A cyclic N-methylated hexapeptide somatostatin analog denoted “Somato 8” (SEQ ID NO: 26) was selected from a combinatorial library of all possible N-methylated analogs of the potent hexa cyclic somatostatin analog c(PFwKTF) (SEQ ID NO: 35) (Veber D F, Freidlinger R M, Perlow D S, et al. Nature 1981; 292(5818):55-8), in an effort to develop an improved somatostatin analog. Out of the 30 analogs synthesized, only seven analogs were found to have somatostatin receptor (SSTR) affinity similar to that of the parent peptide, that is, selectivity towards SSTR2 and SSTR5 in the nanomolar range. From this library, one analog, named “Somatostatin 8”, having the sequence c(-PF(NMe)w(NMe)KT(NMe)F—), that had three N-methyl groups (Somatostatin 8, Scheme U), had the most promising PK parameters in vitro (including stability to intestinal enzymes and intestinal permeability). It was further investigated for its bioavailability following oral administration to rats compared to the parent sequence. The calculated absolute oral bioavailability of the multiple N-methylated analog in rats was ˜10% which is nearly five times higher than the parent peptide [28]. The dihexyloxycarbonyl prodrug of Somatostatin 8, namely Somatostatin 8P (FIG. 24) was prepared in the same way as Octreotide P (FIG. 23).

### Example 9: Synthesis of Prodrug of a Backbone Cyclic Peptide

The novel backbone cyclic somatostatin analog Somato3M (SEQ ID NO: 30) having the three N-methylated active sequence (NMe)w-(NMe)K-T-(NMe)F was used to produce its three hexyloxycbarbonyl prodrug.

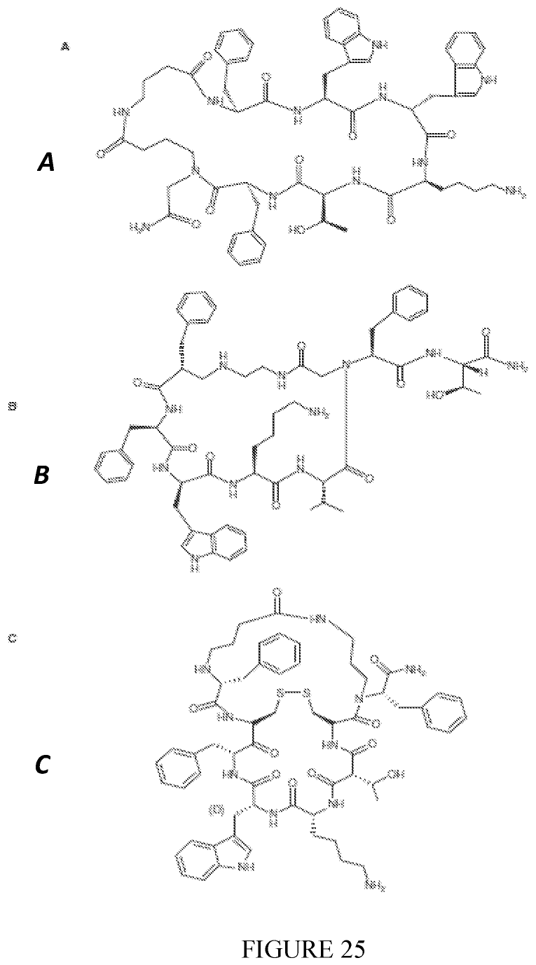

In an attempt to identify novel somatostatin analogs, libraries of backbone cyclic peptides have been previously prepared with compounds having identical or highly similar sequences to the somatostatin pharmacophoric sequences. Four libraries, each containing 96 compounds, were synthesized and screened for their binding affinities to somatostatin receptors. Following the screening process, several candidates were further investigated for their metabolic stability and pharmacodynamic profile compared to SRIF and octreotide. Some of the compounds are PTR-3046 [29], PTR-3205 [30] and PTR-3173 (SEQ ID NO: 27) [31] depicted in FIG. 25.

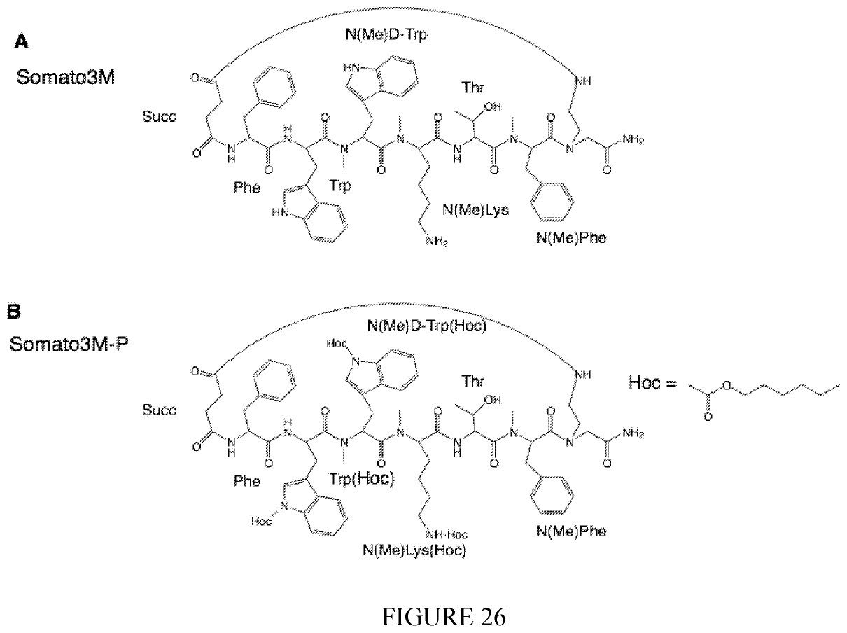

All backbone cyclic analogs were found to be stable against enzymatic degradation in serum and renal homogenate. However, their biological activity and selectivity varied toward the somatostatin receptors: while PTR-3046 was found to be selective toward the SSTR5 (IC50 in the nanomolar range), PTR-3205 was found to be selective towards SSTR2 and PTR-3173 was selective towards the SSTR2, SSTR4 and SSTR5. These analogs were also evaluated for their in vivo efficacy compared to octreotide. PTR-3173 was found to be 1000-fold more potent in the in vivo inhibition of GH than that of glucagon, with no effect on insulin secretion at physiological concentrations (GH: insulin potency ratio >10,000). This was the first description of a long-acting SRIF analog possessing complete in vivo selectivity between GH and insulin inhibition. PTR-3046 inhibits bombesin- and caerulein-induced amylase and lipase release from the pancreas without inhibiting GH or glucagon release. PTR-3173 has been reported to bind uniquely to SSTR2, SSTR4 and SSTR5 in vitro with outstanding in vivo selectivity in GH inhibition [31]. All backbone cyclic analogs were found to be stable against enzymatic degradation in serum and renal homogenate. The active N-methylated sequence (NMe)w-(NMe)K-T-(NMe)F— was incorporated into the framework of the backbone cyclic analog PTR 3173 to form the analog Somato3M, and its three hexyloxycbarbonyl prodrug, namely Somato3M-P (FIG. 26) was prepared in the same manner as Octreotide-P:

The backbone cyclized bridge may be replaced by other types of chemical bridges, e.g, thio-urea, S-amide and by other type and length of connecting groups. Each combination imposes certain pharmacodynamics selectivity towards the somatostatin receptor subtypes. The N-methylation at different sites may elevate intestinal permeability.

The foregoing description of the specific embodiments, will so fully reveal the general nature of the invention that others can, by applying current knowledge, readily modify and/or adapt for various applications such specific embodiments, without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

1. Nomenclature and Symbolism for Amino. Acids and Peptides Recommendations 1983. IUPAC-IUB Jt. Comm.
2. Ovadia O, Greenberg, S, Chatterjee J, Laufer B, Opperer F et al. The Effect of Multiple N-Methylation on Intestinal Permeability of Cyclic Hexapeptides. Mol. Pharmaceutics 2011, 8, 479-487.
3. A. O. Frank, E. Otto, C. Mas-Moruno, H. B. Schiller, L. Marinelli, et al. Conformational control of integrin-subtype selectivity in isoDGR peptide motifs: a biological switch. Angew. Chem. Int.

Ed. 2010, 49, 9278-9281.
4. T. G. Kapp, M. Fottner, O. V. Maltsev, H. Kessler, Small Cause, Great Impact: Modification of the Guanidine Group in the RGD Motif Controls Integrin Subtype Selectivity. Angew. Chem. 2016, 128, 1564
5. Marelli U K, Bezencon J, Puig E, Ernst B, Kessler H. Enantiomeric Cyclic Peptides with Different Caco-2 Permeability Suggest Carrier-Mediated Transport. Chemistry. 2015 May 26; 21(22): 8023-7.
6. Xiong J-P. Crystal Structure of the Extracellular Segment of Integrin alpha Vbeta 3 in Complex with an Arg-Gly-Asp Ligand. Science. 2002; 296:151-5.
7. Takagi J, Strokovich K, Springer T A, Walz T. Structure of integrin alpha5beta1 in complex with fibronectin. EMBO J. 2003; 22:4607-15.
8. Dong X, Zhao B, Iacob R E, Zhu J, Koksal A C, Lu C, et al. Force interacts with macromolecular structure in activation of TGF-β. Nature, 2017; 542:55-9.
9. Springer T A, Zhu J, Xiao T. Structural basis for distinctive recognition of fibrinogen gammaC peptide by the platelet integrin alphaIIbbeta3. J. Cell Biol. 2008; 182:791-800.
10. Kapp T G, Rechenmacher F, Neubauer S, Maltsev O V, Cavalcanti-Adam E A, Zarka R, et al. A Comprehensive Evaluation of the Activity and Selectivity Profile of Ligands for RGD-binding Integrins Sci Rep. 2017 Jan. 11; 7:39805.
11. Bochen A, Kiran Marelli U, Otto E, Pallarola D, Mas-Moruno C, Saverio Di Leva F, et al. Biselectivity of isoDGR Peptides for Fibronectin Binding Integrin Subtypes α5β1 and αvβ6: Conformational Control through Flanking Amino Acids. J. Med. Chem., 2013, 56 (4), pp 1509-1519.
12. van Ryn J, Goss A, Hauel N, Wienen W, Priepke H, Nar H, et al. The discovery of dabigatran etexilate. Front Pharmacol. 2013 Feb. 12; 4:12.
13. Artursson P, Karlsson J. Correlation between oral drug absorption in humans and apparent drug permeability coefficients in human intestinal epithelial (Caco-2) cells. Biochem. Biophys. Res. Commun. 1991; 175:880-5.
14. Kumar K K V, Karnati S, Reddy M B, Chandramouli R. Caco-2 cell lines in drug discovery—an updated perspective. J. basic Clin. Pharm. 2010; 1:63-9.
15. Hubatsch I, Ragnarsson E G E, Artursson P. Determination of drug permeability and prediction of drug absorption in Caco-2 monolayers. Nat. Protoc. 2007; 2:2111-9.
16. Artursson P. Epithelial transport of drugs in cell culture. I: A model for studying the passive diffusion of drugs over intestinal absorptive (Caco-2) cells. J. Pharm. Sci. 1990; 79:476-82.
17. Hunter J, Hirst B H, Simmons N L. Drug absorption limited by P-glycoprotein-mediated secretory drug transport in human intestinal epithelial Caco-2 cell layers. Pharm Res. 1993. p. 743-9.
18. Picariello G, Addeo F. Use of brush border membrane vesicles to simulate the human intestinal digestion. Food Res. Int. 2016; 88:327-35.
19. Powell M F. Chapter 30. Peptide Stability in Drug Development: in vitro Peptide Degradation in Plasma and Serum. Annual Reports in Medicinal Chemistry Volume 28, 1993, Pages 285-294.
20. P. P. Wong, N. Bodrug, K. M. Hodivala-Dilke, Exploring novel methods for modulating tumor blood vessels in cancer treatment Curr. Biol. 2016, 26, 1161-1166.
21. J. P. Xiong, T. Stehle, R. G. Zhang. A. Joachimiak, M. Frech, S. L. Goodman, M. A. Crystal structure of the complete integrin αVβ3 ectodomain plus an α/β transmembrane fragment Arnaout, Science 2002, 296, 151-155.
22. G. M. Sastry, M. Adzhigirey, T. Day, R. Annabhimoju, W. Sherman, J. Protein and ligand preparation: parameters, protocols, and influence on virtual screening enrichments. Comput. Aid. Mol. Des. 2013, 27, 221-234.
23. J. R. Greenwood, D. Calkins, A. P. Sullivan, J. C. Shelley, J. Comput. Towards the comprehensive, rapid, and accurate prediction of the favorable tautomeric states of drug-like molecules in aqueous solution Aided Mol. Des. 2010, 24, 591-604.
24. J. C. Shelley, A. Cholleti, L. Frye, J. R. Greenwood, M. R. Timlin, M. Uchimaya, J. Epik: a software program for pK(a) prediction and protonation state generation for drug-like molecules Comput. Aided Mol. Des. 2007, 21, 681-691.
25. R. A. Friesner, J. L. Banks, R. B. Murphy, T. A. Halgren, J. J. Klicic, et al. Glide: a new approach for rapid, accurate docking and scoring. 1. Method and assessment of docking accuracy J. Med. Chem. 2004, 47, 1739-1749.
26. T. A. Halgren, R. B. Murphy, R. A. Friesner, H. S. Beard, L. L. Frye, et al. Glide: a new approach for rapid, accurate docking and scoring. 2. Enrichment factors in database screening. J. Med. Chem. 2004, 47, 1750-1759.
27. E. Harder, W. Damm, J. Maple, C. Wu, M. Reboul, et al. OPLS3: A Force Field Providing Broad Coverage of Drug-like Small Molecules and Proteins J. Chem. Theory Comput. 2016, 12, 281-296.
28. Biron E, Chatterjee J, Ovadia O, Langenegger D, Brueggen J, Hoyer D, et al. Improving Oral Bioavailability of Peptides by Multiple N-Methylation: Somatostatin Analogues.
29. Chaim Gilon ‡, Martin Huenges §, Barbara Mathä §, Gary Gellerman ⊥, Vered Hornik ⊥, Michel Afargan ⊥, et al. A Backbone-Cyclic, Receptor 5-Selective Somatostatin Analogue: Synthesis, Bioactivity, and Nuclear Magnetic Resonance Conformational Analysis†. American Chemical Society; 1998;
30. Falb E, Salitra Y, Yechezkel T, Bracha M, Litman P, Olender R, et al. A bicyclic and hsst2 selective somatostatin analogue: design, synthesis, conformational analysis and binding. Bioorg. Med. Chem. 2001; 9:3255-64.
31. Afargan M, Janson E T, Gelerman G, Rosenfeld R, Ziv O, Karpov O, et al. Novel Long-Acting Somatostatin Analog with Endocrine Selectivity: Potent Suppression of Growth Hormone But Not of Insulin. Endocrinology. 2001; 142:477-86.

1-45. (canceled)

46. A peptide-based prodrug comprising at least one carbamate moiety, wherein said at least one carbamate moiety is having a formula selected from the group consisting of 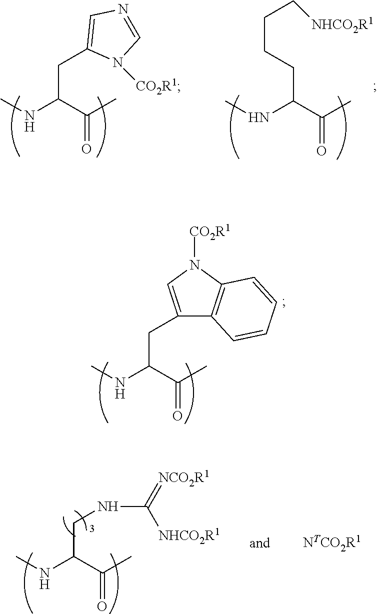
wherein
R¹ is an unsubstituted primary C₃₋₄₀ alkyl; and
N^{T} is an N-terminus nitrogen atom of the peptide sequence of said peptide-based prodrug.

47. The peptide-based prodrug of claim 46, wherein R¹ is n-C₆H₁₃.

48. The peptide-based prodrug of claim 46, which is a cyclic peptide based prodrug, wherein the cyclic peptide based prodrug is somatostatin or a somatostatin analog.

49. The peptide-based prodrug of claim 46, which is devoid of positively charged nitrogen atoms.

50. The peptide-based prodrug of claim 46, wherein the carbamate moiety has a formula selected from the group consisting of:

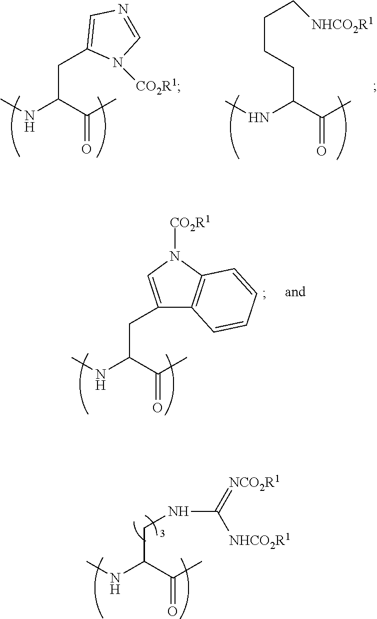

51. The peptide-based prodrug of claim 46, prepared by a process comprising:
(a) providing a peptide; and
(b) reacting said peptide with an alkyl haloformate having the formula XCO₂R¹, wherein X is a halogen, thereby forming the peptide-based prodrug.

52. The peptide-based prodrug of claim 46, wherein the process further comprises a step of reacting the peptide of step (a) or the peptide-based prodrug of step (b) with an alcohol in the presence of an esterification reagent.

53. The peptide-based prodrug of claim 46, wherein the carbamate moiety has the formula:

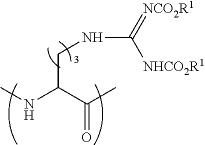

54. A process for preparing a peptide-based prodrug, the process comprising
(a) providing a peptide precursor;
(b) coupling said peptide precursor with a modified amino acid having a formula selected from the group consisting of 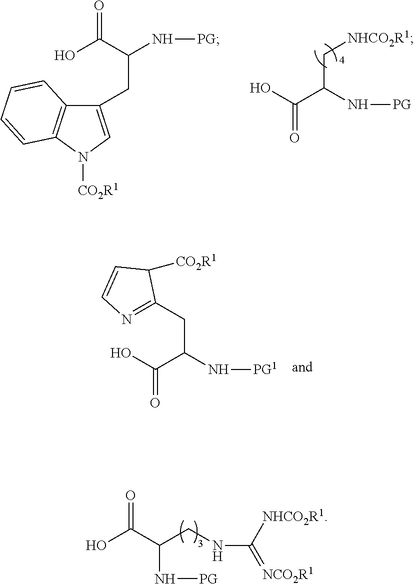
wherein
R¹ is a primary alkyl,
PG is a protecting group,
wherein the peptide precursor is selected from the group consisting of: an amino acid, a peptide and a solid phase resin.
(c) removing said protecting group PG¹ from the product of step (b); and
(d) optionally coupling at least one additional amino acid;
thereby forming the peptide-based prodrug.

55. The process of claim 54, wherein the modified amino acid is having a formula selected from the group consisting of:

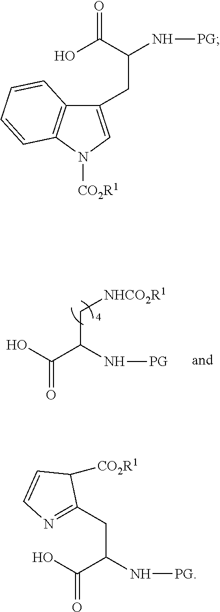

56. The process of claim 54, wherein the modified amino acid is having the formula:

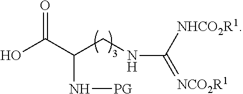

57. The process of claim 54, further comprising a step of reacting the product of step (c) or (d) with an alkyl chloroformate having the formula ClCO₂R¹.

58. The process of claim 54, wherein R¹ is n-C₆H₁₃.

59. The process of claim 54, wherein the peptide-based prodrug is devoid of positively charged atoms.

60. A process for preparing a peptide-based prodrug, the process comprising
(a) providing a peptide precursor;
(b) coupling said peptide precursor with a protected amino acid having a formula selected from the group consisting of 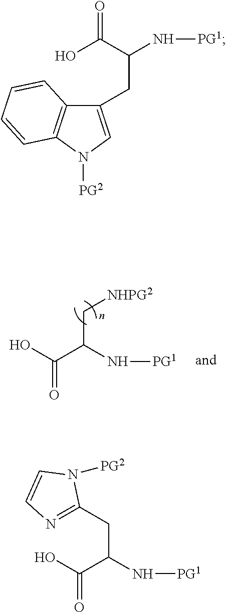
wherein
PG¹ is a base-labile protecting group,
PG² is an acid-labile protecting group,
n is 3 or 4, and
wherein the peptide precursor is selected from the group consisting of: an amino acid, a peptide and a solid phase resin;
(c) removing said acid-labile protecting group PG² from the product of step (b) under acidic conditions;
(d) reacting the product of step (c) with a compound selected from 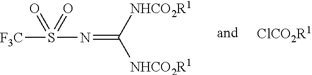
wherein R¹ is a primary alkyl;
(e) removing said base-labile protecting group under basic conditions; and
(f) optionally coupling at least one additional amino acid,
thereby forming the peptide-based prodrug.

61. The process of claim 60, wherein the protected amino acid is having the formula 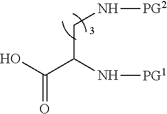
and wherein the reaction of step (d) is with a compound having the formula

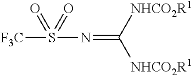

62. The process of claim 60, wherein the protected amino acid is having the formula selected from the group consisting of: 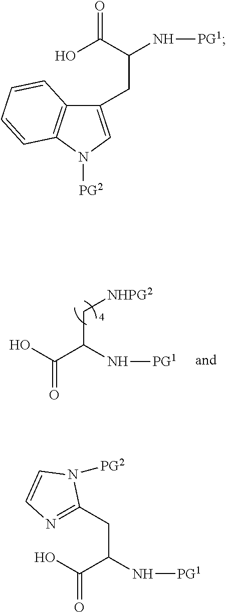
and wherein the reaction of step (d) is with a compound having the formula ClCO₂R¹.

63. The process of any one of claim 60, wherein R¹ is n-C₆H₁₃.

64. The process of any one of claim 60, wherein the peptide-based prodrug is devoid of charged atoms.

65. The process of any one of claim 60, further comprising step (g) of reacting the peptide-based prodrug with an alcohol in the presence of thionyl chloride.


SEQUENCE LISTING (35 sequences)

SEQ ID NO: 1 - protein, 6 aa, Artificial Sequence
  Cyclic peptide
  MISC_FEATURE (1)..(1): D-amino acid
  MISC_FEATURE (1)..(1): N-methylated
  MISC_FEATURE (5)..(5): N-methylated
RGDAAA

SEQ ID NO: 2 - protein, 6 aa, Artificial Sequence
  cyclic peptide
  MISC_FEATURE (1)..(1): D-amino acid
  MISC_FEATURE (1)..(1): N methylated
  MISC_FEATURE (6)..(6): N methylated
ARGDAA

SEQ ID NO: 3 - protein, 6 aa, Artificial Sequence
  Cyclic peptide
  MISC_FEATURE (1)..(1): D-amino acid
  MISC_FEATURE (3)..(3): N methylated
  MISC_FEATURE (5)..(5): N methylated
RGDAAA

SEQ ID NO: 4 - protein, 6 aa, Artificial Sequence
  Cyclic peptide
  MISC_FEATURE (1)..(1): D-amino acid
  MISC_FEATURE (5)..(5): N methylated
  MISC_FEATURE (6)..(6): N methylated
RGDAAA

SEQ ID NO: 5 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
VRGDAA

SEQ ID NO: 6 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
FRGDAA

SEQ ID NO: 7 - protein, 6 aa, Artificial Sequence
  Cyclic peptide
  MISC_FEATURE (1)..(1): D-amino acid
  MISC_FEATURE (1)..(1): N methylated
  MISC_FEATURE (5)..(5): N methylated
RGDAAV

SEQ ID NO: 8 - protein, 6 aa, Artificial Sequence
  Cyclic peptide
  MISC_FEATURE (1)..(1): D-amino acid
  MISC_FEATURE (1)..(1): N methylated
  MISC_FEATURE (5)..(5): N methylated
RGDAAF

SEQ ID NO: 9 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
VRGDAA

SEQ ID NO: 10 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
ARGDAA

SEQ ID NO: 11 - protein, 6 aa, Artificial Sequence
  Cyclic peptide
  MISC_FEATURE (1)..(1): D-amino acid
  MISC_FEATURE (1)..(1): N methylated
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
RGDAAA

SEQ ID NO: 12 - protein, 6 aa, Artificial Sequence
  Cyclic peptide
  MISC_FEATURE (1)..(1): D-amino acid
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
  MISC_FEATURE (6)..(6): N methylated
RGDAAA

SEQ ID NO: 13 - protein, 6 aa, Artificial Sequence
  Cyclic peptide
  MISC_FEATURE (1)..(1): D-amino acid
  MISC_FEATURE (1)..(1): Two hexyloxycarbonyl (Hoc) moieties
  MISC_FEATURE (3)..(3): N methylated
  MISC_FEATURE (3)..(3): Methyl ester (OMe) moiety
  MISC_FEATURE (5)..(5): N metylated
RGDAAA

SEQ ID NO: 14 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
FRGDAA

SEQ ID NO: 15 - protein, 9 aa, Artificial Sequence
  Cyclic peptide
  MISC_FEATURE (5)..(5): Two hexyloxycarbonyl (Hoc) moieties
LPPFRGDLA

SEQ ID NO: 16 - protein, 8 aa, Artificial Sequence
  Cyclic peptide
  MISC_FEATURE (6)..(6): Two hexyloxycarbonyl (Hoc) moieties
LPPGLRGD

SEQ ID NO: 17 - protein, 9 aa, Artificial Sequence
  Cyclic peptide
LPPFRGDLA

SEQ ID NO: 18 - protein, 8 aa, Artificial Sequence
  Cyclic peptide
LPPGLRGD

SEQ ID NO: 19 - protein, 6 aa, Artificial Sequence
  Cyclic peptide
  MISC_FEATURE (1)..(1): D-amino acid
AAAAAA

SEQ ID NO: 20 - protein, 5 aa, Artificial Sequence
  Cyclic peptide
  MISC_FEATURE (1)..(1): D-amino acid
  MISC_FEATURE (2)..(2): N methylated
FVRGD

SEQ ID NO: 21 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Hexyloxycarbonyl (Hoc) moiety
  MISC_FEATURE (4)..(4): Methyl ester (OMe) moiety
  MISC_FEATURE (6)..(6): N methylated
VRGDAA

SEQ ID NO: 22 - protein, 6 aa, Artificial sequence
  Cyclic peptide
  MISC_FEATURE (1)..(1): D-amino acid
  MISC_FEATURE (2)..(2): N methylayed
  MISC_FEATURE (3)..(3): Two hexyloxycarbonyl (Hoc) moieties
AAAAAA

SEQ ID NO: 23 - protein, 5 aa, Artificial sequence
  Cyclic peptide
  MISC_FEATURE (1)..(1): D-amino acid
  MISC_FEATURE (2)..(2): N methylated
  MISC_FEATURE (3)..(3): Two hexyloxycarbonyl (Hoc) moieties
FVRGD

SEQ ID NO: 24 - protein, 9 aa, Artificial sequence
  Peptide
RTDLDSLRT

SEQ ID NO: 25 - protein, 8 aa, Artificial Sequence
  Synthetic peptide
  MISC_FEATURE (1)..(1): D-Phe
  DISULFID (2)..(7): bridge
  MISC_FEATURE (4)..(4): D-Trp
  MISC_FEATURE (8)..(8): (ol) = alcohol C termuinus
FCFWKTCT

SEQ ID NO: 26 - protein, 6 aa, Artificial Sequence
  Synthetic peptide
  MOD_RES (1)..(6): head-to-tail cyclization
  MOD_RES (3)..(3): N-methyl D-Trp
  MOD_RES (4)..(4): N-methyl Lys
  MOD_RES (6)..(6): N-methyl Phe
PFWKTF

SEQ ID NO: 27 - protein, 8 aa, Artificial Sequence
  Synthetic peptide
  MOD_RES (1)..(1): 4Abu, GABA
  MOD_RES (1)..(8): backbone-to-end cyclization between the N-alpha-omega-functionalized derivative of GlyC3 and the N-terminus
  MOD_RES (4)..(4): D-Trp
  MOD_RES (8)..(8): AMIDATION
  MOD_RES (8)..(8): GlyC3 building unit
XFWWKTFX

SEQ ID NO: 28 - protein, 7 aa, Artificial Sequence
  Synthetic peptide
  MOD_RES (1)..(1): PheN2 building unit
  MOD_RES (1)..(6): backbone cyclization between the N-alpha- omega-functionalized derivative of X1 and the N-alpha-omega- functionalized derivative of X6
  MOD_RES (3)..(3): D-Trp
  MOD_RES (6)..(6): PheC3 building unit
  MOD_RES (7)..(7): AMIDATION
FYWKVFT

SEQ ID NO: 29 - protein, 9 aa, Artificial Sequence
  Synthetic peptide
  MOD_RES (1)..(1): PheC3 building unit
  MOD_RES (1)..(9): backbone cyclization between the N-alpha-omega- functionalized derivative of X1 and the N-alpha-omega- functionalized derivative of X9
  DISULFID (2)..(7): bridge
  MOD_RES (4)..(4): D-Trp
  MOD_RES (9)..(9): PheN3 building unit
  MOD_RES (9)..(9): AMIDATION
FCFWKTCFF

SEQ ID NO: 30 - protein, 6 aa, Artificial Sequence
  Synthetic peptide
  MOD_RES (1)..(6): head-to-tail cyclization
  MOD_RES (3)..(3): N-methyl D-Trp
  MOD_RES (4)..(4): N-methyl Lys
  MOD_RES (6)..(6): N-methyl Phe
FWWKTF

SEQ ID NO: 31 - protein, 9 aa, Artificial Sequence
  Synthetic peptide
  DISULFID (1)..(6): bridge
  MOD_RES (9)..(9): AMIDATION
CYIQNCPLG

SEQ ID NO: 32 - protein, 9 aa, Artificial Sequence
  Synthetic peptide
  DISULFID (1)..(6): bridge
  MOD_RES (9)..(9): hexyloxycarbonyl (Hoc) moiety
CYIQNCPLG

SEQ ID NO: 33 - protein, 9 aa, Artificial Sequence
  Synthetic peptide
  MOD_RES (2)..(2): Two hexyloxycarbonyl (Hoc) moieties
  MOD_RES (4)..(9): cyclization
GRPCNQFYC

SEQ ID NO: 34 - protein, 9 aa, Artificial Sequence
  Synthetic peptide
  MOD_RES (2)..(2): AMIDATION
  MOD_RES (4)..(9): cyclization
GRPCNQFYC

SEQ ID NO: 35 - protein, 6 aa, Artificial Sequence
  Synthetic peptide
  MOD_RES (1)..(6): head-to-tail-cyclization
  MOD_RES (3)..(3): D-Trp
PFWKTF